(19) United States
(12) Patent Application Publication
(10) Pub. No.: US 2005/0004044 A1
(43) Pub. Date: Jan. 6, 2005

(54) Use of orsaponin [3beta, 16beta, 17 alpha-trihydroxycholost-5-en-22-one 16-0-(2-0-4-methoxybenzoyl-beta-D-xylopyranosyl)-(1->3)-(2-0-acetyl-alpha-L-arabinopyranoside)] or OSW-1 and its derivatives for cancer therapeutics

(75) Inventors: Peng Huang, Bellaire, TX (US); Michael Keating, Houston, TX (US); Zhendong Jin, Coralville, IA (US)

Correspondence Address: FULBRIGHT & JAWORSKI L.L.P., 600 CONGRESS AVE., SUITE 2400, AUSTIN, TX, 78701, US

(73) Assignees: Board of Regents; The University of Texas System; The University of Iowa Research Foundation

(21) Appl. No.: 10/819,479

(22) Filed: Apr. 7, 2004

Related U.S. Application Data
(60) Provisional application No. 60/460,946, filed on Apr. 7, 2003.

(51) Int. Cl. A61K 31/704; A61K 31/56

(52) U.S. Cl. 514/26; 514/169

(57) ABSTRACT

The present invention concerns methods for treating pancreatic cancers, leukemias, colon cancers, malignant gliomas and other brain tumors, and ovarian cancers which comprise providing to an individual compositions comprising an orsaponin such as OSW-1 or its derivatives such as 17-deoxyorsaponin. Various therapeutically useful derivatives of orsaponins are also described.

### BACKGROUND OF THE INVENTION

The present invention claims the benefit of the filing date of co-pending U.S. Provisional Patent Application Ser. No. 60/460,946 filed on Apr. 7, 2003. The entire text of the above-referenced disclosure is specifically incorporated herein by reference without disclaimer.

I. Field of the Invention

The present invention relates generally to the fields of cancer and biochemistry. More particularly, it concerns methods for treating and preventing pancreatic cancers, chronic lymphocytic leukemia (CLL), colon cancers, and ovarian cancers by the administration of compositions comprising orsaponins to individuals afflicted with such cancers.

II. Description of Related Art

A. Cancers

Pancreatic cancer is the fourth leading cause of cancer death in men and women in America. The American Cancer Society estimates that, in 2003, about 30,700 people in the United States will be found to have pancreatic cancer, and about 30,000 will die of the disease. Fewer than 5% of all patients diagnosed with pancreatic cancer can expect to survive 5 years. About 2 out of 10 patients with cancer of the pancreas will live at least 1 year after the cancer is found, but only a very few will survive for 5 years. Not much is still known about the mechanisms of pancreatic cancer. This type of cancer produces few specific symptoms until late in the disease, so it usually proceeds ‘silently’ and often is unnoticed until it is terminal. It is also one of the most biologically aggressive solid tumors with an enormous potential to invade and spread very early. At the time of diagnosis, patients usually have locally advanced or metastatic disease to the lymph nodes, liver, lungs and peritoneum (Evans et al., 1997; Korc, 1998). The use of traditional chemotherapy and radiation has generated only modest improvements in outcome after resection and likewise has offered little hope to those individuals with unresectable disease (Jacobson et al., 1997).

The same is true for chronic lymphocytic leukemia (CLL) which are difficult to treat. Chronic lymphocytic leukemia (CLL), mainly affects a type of lymphocyte called the B lymphocytes and in some cases affects T lymphocytes, and causes suppression of the immune system, failure of the bone marrow, and infiltration of malignant cells into organs. Although leukemia starts in the bone marrow, it can spread to the blood, lymph nodes, spleen, liver, central nervous system (CNS) and other organs. Treatment options for CLL depend on the disease stage. High-risk CLL and intermediate-risk CLL are typically treated with chemotherapeutic agents such as chlorambucin, cyclophosphamide or fludarabine. However, the average survival for patients with high risk CLL is only about 4 years and about 7 years for those with intermediate-risk disease. In addition, CLLs of different origins have different clinical presentations and disease courses. B-cell CLLs generally infiltrate the lymph nodes, bone marrow, and spleen and tend to have an indolent course. In contrast, T-cell CLLs are more malignant and present additional infiltration in the skin (Freedman et al., 1990).

Treatment of CLL is generally individualized. No specific treatment is required in older patients having an indolent form of the disease. However, other patients with more advanced disease or with disease having a more rapid course may have a median survival of less than two years. Therefore, appropriate treatment should be pursued. The majority of patients have an intermediate prognosis, and although they fare reasonably well without treatment for several years, ultimately they will require some form of therapy. The typical treatment for CLL is the administration of chlorambucil, a chemotherapeutic agent. Combination chemotherapy is generally used only in advanced cases. Radiation therapy has been effectively used, particularly if splenic enlargement is present and bone marrow transplantation has been successful with younger patients (Foon et al., 1992). More recently, the nucleoside fludarabine, a fluorinated adenine analog, and 2-chlorodeoxyadenosine, a deoxyadenosine analog, have been found to be effective. Thus, there is a need in the art to develop better therapeutic regimens for CLL.

Ovarian cancer is the sixth most common cancer in women. It ranks fifth as the cause of cancer death in women. The American Cancer Society estimates that there will be about 25,400 new cases of ovarian cancer in the United States alone in 2003. About 14,300 women are estimated to die of the disease. The chances of survival from ovarian cancer are better if the cancer is found early. If the cancer is found and treated before it has spread outside the ovary, 95% of women will survive at least five years. However, only 25% of ovarian cancers are found at this early stage. About 78% of all women with ovarian cancer survive at least one year after the cancer is found, and over half survive longer than five years. Again, given this background, new forms of cancer therapy are required to improve treatment outcomes of ovarian cancers.

Colon cancer is the second most frequently diagnosed malignancy in the United States. For localized colon cancer, surgery is the primary treatment and results in a cure rate of approximately 50% of patients. Recurrence following surgery is a major problem, and often is the cause of patient death. Adjuvant therapy with chemotherapeutic agents such as 5-FU and leucovorin plays a role in the treatment of colon cancer and benefits the cancer patients to certain degree. The prognosis of colon cancer is closely associated with the extent of local tumor penetration and distal metastasis. Elevated serum levels of carcinoembryonic antigen (CEA) is also a negative prognostic significance. Because colon cancer is the second most common cause of cancer death, the development of more effective anticancer agents for the treatment of colon cancer is urgently needed.

B. Genetic Factors Involved in Cancer

Various genetic components have been associated with cancers. One such factor is NFκB which is an important transcriptional factor that is involved in the regulation of gene expression in cells. It is known in the art that NFκB activation is associated with the development of certain cancers, especially pancreatic cancer. As the activation of NFκB can protect cancer cells from apoptosis it is likely that NFκB contributes to the development of drug resistance. Thus, anticancer agents that can kill or inhibit the growth of cancer cells which constitute NFκB activation are much sought after in the art.

The tumor suppressor gene p53 is also a transcription factor with multiple biological functions. Mutation of p53 or a defect in p53 functional pathway is often associated with tumor development. In fact, it is known in the art that over 50% of human cancers carry some form of p53 mutations. Because the normal p53 molecular function is important in the cellular apoptotic response to many anticancer agents with DNA-damaging properties, loss of p53 function due to mutations or other defects causes a failure in apoptotic response, and thus contributes to drug resistance. Thus, anticancer drugs that effectively kill cancer cells with p53 mutations are also much sought after.

C. Chemotherapeutics

Several plant based chemotherapeutic agents have been used in the art for the treatment of cancers. Of these, an orsaponin called [3β, 16β, 17 α-trihydroxycholost-5-en-22-one 16-O-(2-O-4-methoxybenzoyl-β-D-xylopyranosyl)-(1->3)-(2-O-acetyl-α-L-arabinopyranoside)] also known more commonly as OSW-1 is reported to have anti-tumor activities. For example, Mimaki et al. (1997), have shown that OSW-1 suppresses growth of the leukemia cell line HL-60 with an IC₅₀ of 0.1-0.3 nM and that it has potent cytostatic activities on other malignant tumor cell lines including human leukemia cells (CCRF-CEM), mouse mastocarcinoma cells (FM3A), human pulmonary adenocarcinoma cells (A-549), human pulmonary large cell carcinoma cells (Lu-65, and Lu-99), human pulmonary squamous cell carcinoma cells (RERF-LC-A1), adriamycin-resistant P388 leukemia cells and camptothecin-resistant P388 cells. OSW-1 was also found to be cytostatic in the U.S. NCI 60-cell in vitro screen and melanoma cells were particularly sensitive to OSW-1. The present inventors and other groups have synthesized derivatives of OSW-1 and shown that these derivatives also have cytostatic activities against cancer cells (Yu, 2001; Yu, 2002; Kuroda et al., 2001; Ma et al., 2000, 2001a, 2001b).

However, the efficacy of the orsaponins and OSW-1 in particular has not been tested in in vivo models of cancer. There is also a need to determine the effect of orsaponins on other types cancers, especially in cancers that have an abnormal activation of NFκB, and/or a defect in p53 function.

### SUMMARY OF THE INVENTION

The present invention overcomes the existing defects in the art and provides compositions comprising one or more orsaponins that are effective in the treatment and prevention of pancreatic cancers, CLL, colon cancers, and ovarian cancers. Orsaponins can be isolated from plants or synthesized by different methods. Co-pending U.S. Patent Application Publication No. US 2003/0069214, the entire disclosure of which is incorporated herein by reference, describes methods for the synthesis of the orsaponin Osw-1.

Thus, in some embodiments, the present invention provides methods of treating a human with a pancreatic cancer, a chronic lymphocytic leukemia (CLL), a colon cancer, a malignant glioma or brain tumor, or an ovarian cancer comprising administering a therapeutically effective amount of a pharmaceutical composition comprising orsaponin or derivatives thereof wherein said orsaponin has the molecular formula: 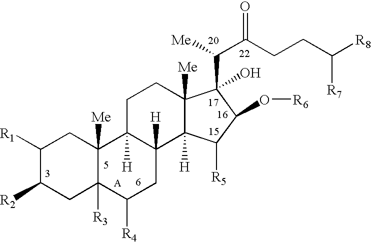
wherein,
R₁ is a H, an OH, or an MeO, with either an R or an S stereochemistry,
R₂ is a H, an OH, an ester or an amide,
R₃ is H, OH, or forms part of the double-bond A,
R₄ is H, OH, or forms part of the double-bond A,
R₅ is a H, a disaccharide, a monosaccharide or a trisaccharide,
R₆ is a disaccharide, a monosaccharide or a trisaccharide,
R₇ is a Me, a C₁₋₁₂ alkyl, or preferably a C₂₋₆ alkyl, or a phenyl,
R₈ is a Me, a C₁₋₁₂ alkyl, or preferably a C₂₋₆ alkyl, or a phenyl, and C₂₀ is an S or an R isomer,
   or is a stereoisomer thereof.

In other embodiments, the method is further defined as a method of preventing cancer.

An “effective amount” is defined as an amount of the orsaponin composition that will decrease, reduce, inhibit or otherwise abrogate the growth of a cancer cell, arrest-cell growth, induce apoptosis, inhibit metastasis, induce tumor necrosis, kill cells or induce cytotoxicity in cells. In some embodiments, the therapeutically effective amount is 0.5-50 μg/kg/day. In yet other embodiments, the therapeutically effective amount is 1-10 μg/kg/day. Thus, it is contemplated that one may use 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day, 10 μg/kg/day, 15 μg/kg/day, 20 μg/kg/day, 30 μg/kg/day, 40 μg/kg/day, or 50 μg/kg/day. Of course, intermediate ranges, such as 0.75 μg/kg/day, 1.5 μg/kg/day, 5.5 μg/kg/day, 12.5 μg/kg/day and the like are also contemplated. As will be recognized by the skilled artisan, the final dosage administered to a patient will be subject to further adjustments based on specific disease conditions, age, gender, and other health conditions of each individual patient, and such dose adjustments will be performed by a trained physician at the time of treatment. The present invention is therefore not limited by the dose related adjustments.

In one specific embodiment the orsaponin is OSW-1 and has the molecular formula:

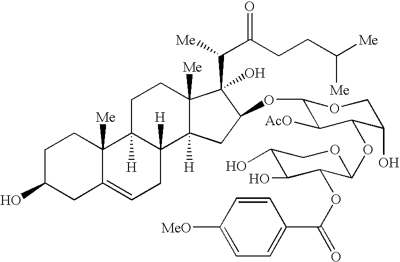

In some specific embodiments, the pancreatic cancer, chronic lymphocytic leukemia (CLL), colon cancer, or ovarian cancer is a drug-resistant cancer. In other specific embodiments, the pancreatic cancer, the chronic lymphocytic leukemia (CLL) cancer, the colon cancer, or the ovarian cancer is a metastatic cancer. In yet other specific embodiments, the cancer comprises cells that express or over-express NFκB, or has a p53 mutation or defect.

The methods of the invention are useful for the treatment of any pancreatic cancer and in some non-limited embodiments the pancreatic cancer is a ductal adenocarcinoma, a mucinous cystadenocarcinoma, an acinar carcinoma, an unclassified large cell carcinoma, a small cell carcinoma, an intraductal papillary neoplasm, a mucinous cystadnoma, a papillary cystic neoplasm, or a pancreatoblastoma.

The methods of the invention are also useful for the treatment of any ovarian cancer. Some non-limiting examples of ovarian cancer include ovarian carcinoma, a serous cell cancer, a mucinous cell cancer, an endometrioid cell cancer, a clear cell cancer, a mesonephroid cell cancer, a Brenner cell cancer, or a mixed epithelial cell cancer.

Different forms of CLL may also be treated by the methods of the invention. These include T-cell CLL, B-cell CLL, either sensitive or refractory to conventional chemotherapy, as non-limiting examples.

The methods of the invention are also useful for the treatment of cancers of the colon and rectum. Some non-limiting examples of these types of cancer include adenocarcinomas of the colon and rectum such as mucinous adenocarcinoma, adenocacinoma with signet ring features, and squamous cell carcinoma of the rectum.

The orsaponin composition may be administered systemically, regionally or locally. Administration of orsaponin composition can be accomplished by one of several routes including intravenous, intraartetial, intraperitoneal, intradermal, intratumoral, intramuscular, subcutaneous, oral, dermal, nasal, buccal, rectal, vaginal, inhalation, or topical administration.

In some embodiments, the method of the invention further comprises treating the human with a second anti-cancer agent. A variety of cancer therapeutic agents are known in the art and the invention contemplates the use of any of these agents. In some examples, the second agent is a chemotherapeutic agent, a therapeutic antibody, a therapeutic polypeptide, a nucleic acid encoding a therapeutic polypeptide, a therapeutic nucleic acid encoding an antisense, a ribozyme or a RNA, a hormonal agent, an immunotherapeutic agent, or a radiotherapeutic agent. Other adjunct cancer therapies such as surgery, tumor resection, heat therapies, hormonal therapy, etc., are also contemplated.

It is contemplated that in some embodiments, the second agent will be administered simultaneously with the orsaponin composition. In other embodiments, the second agent will be administered prior to administration of the orsaponin composition.

In yet other embodiments, the second agent will be administered after administration of the orsaponin composition.

The invention also provides methods of inducing cytotoxicity in a pancreatic, a chronic lymphocytic leukemia (CLL) cell, a colon cancer cell, or an ovarian cancer cell, comprising contacting the cell with a pharmaceutical composition of orsaponin or a derivative thereof wherein the orsaponin has the molecular formula: 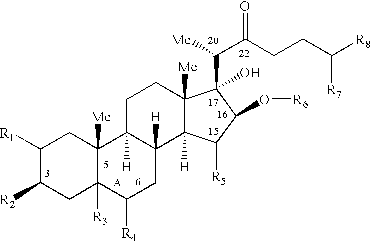
wherein,
R₁ is a H, an OH, or an MeO, with either an R or an S stereochemistry,
R₂ is a H, an OH, an ester or an amide,
R₃ is H, OH, or forms part of the double-bond A,
R₄ is H, OH, or forms part of the double-bond A,
R₅ is a H, a disaccharide, a monosaccharide or a trisaccharide,
R₆ is a disaccharide, a monosaccharide or a trisaccharide,
R₇ is a Me, a C₁₋₁₂ alkyl, or preferably a C₂₋₆ alkyl,
R₈ is a Me, a C₁₋₁₂ alkyl, or preferably a C₂₋₆ alkyl,
and C₂₀ is an S or an R isomer,
   or is a stereoisomer thereof.

In some aspects, the pancreatic cancer cell, the chronic lymphocytic leukemia (CLL) cell, the colon cancer cell, or the ovarian cancer cell is a metastatic cell, or a drug resistant cell or a cancer cell that expresses NFκB.

In some embodiments, the orsaponin composition has an IC₅₀ of 0.1-10 nM and preferably an IC₅₀ of 0.1-5 nM, more preferably an IC₅₀ of 0.1-1 nM, and even more preferably an IC₅₀ of less than 1 nM.

In some aspects of the invention, the orsaponin or derivative thereof induces apoptosis. In yet other aspects, the orsaponin or a derivative thereof kills a pancreatic cancer cell, a chronic lymphocytic leukemia (CLL) cell, a colon cancer cell, or an ovarian cancer cell.

Further provided are methods of inhibiting cell division of a pancreatic cancer cell, a chronic lymphocytic leukemia (CLL) cell, a colon cancer cell, or an ovarian cancer cell, comprising contacting the cell with a pharmaceutical composition comprising orsaponin or a derivative thereof wherein said orsaponin has the molecular formula: 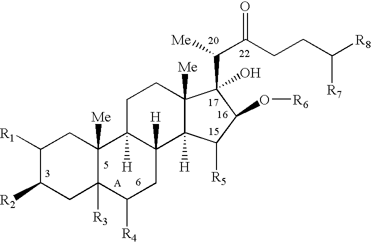
wherein,
R₁ is a H, an OH, or an MeO, with either an R or an S stereochemistry,
R₂ is a H, an OH, an ester or an amide,
R₃ is H, OH, or forms part of a double-bond A,
R₄ is H, OH, or forms part of a double-bond A,
R₅ is a H, a disaccharide, a monosaccharide or a trisaccharide,
R₆ is a disaccharide, a monosaccharide or a trisaccharide,
R₇ is a Me, a C₁₋₁₂ alkyl, or preferably a C₂₋₆ alkyl,
R₈ is a Me, a C₁₋₁₂ alkyl, or preferably a C₂₋₆ alkyl,
and C₂₀ is an S or an R isomer,
   or is a stereoisomer thereof.

The invention also provides methods of inhibiting the growth of a pancreatic cancer cell, a chronic lymphocytic leukemia (CLL) cell, a colon cancer cell, or an ovarian cancer cell, comprising contacting the cell with a pharmaceutical composition comprising orsaponin or a derivative thereof wherein the orsaponin has the molecular formula: 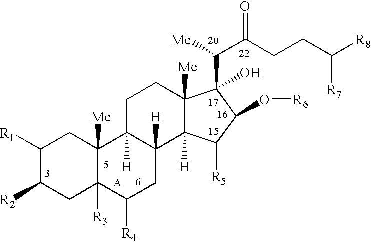
wherein,
R₁ is a H, an OH, or an MeO, with either an R or an S stereochemistry,
R₂ is a H, an OH, an ester or an amide,
R₃ is H, OH, or forms part of a double-bond A,
R₄ is H, OH, or forms part of a double-bond A,
R₅ is a H, a disaccharide, a monosaccharide or a trisaccharide,
R₆ is a disaccharide, a monosaccharide or a trisaccharide,
R₇ is a Me, a C₁₋₁₂ alkyl, or preferably a C₂₋₆ alkyl,
R₈ is a Me, a C₁₋₁₂ alkyl, or preferably a C₂₋₆ alkyl,
and C₂₀ is an S or an R isomer,
   or is a stereoisomer thereof.

In some aspects of the invention, the growth is metastatic growth.

In other specific embodiments, some non-limiting examples of the therapeutic orsaponin compounds of the invention as described in the claims above are set forth below: 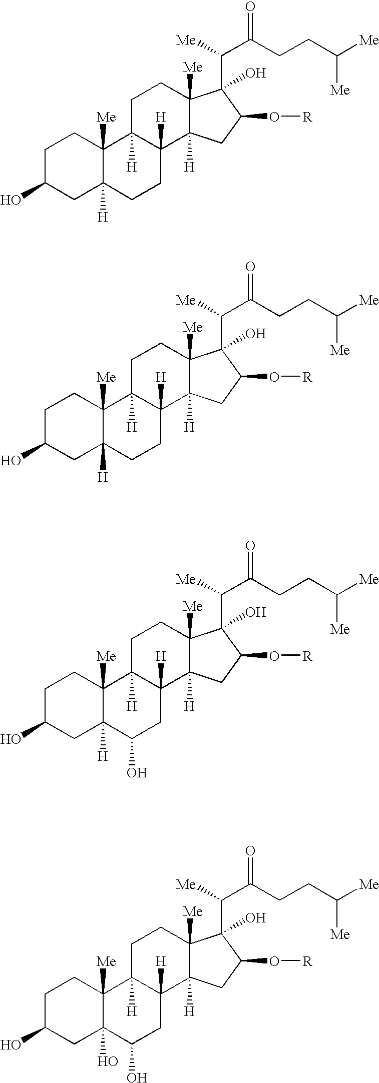 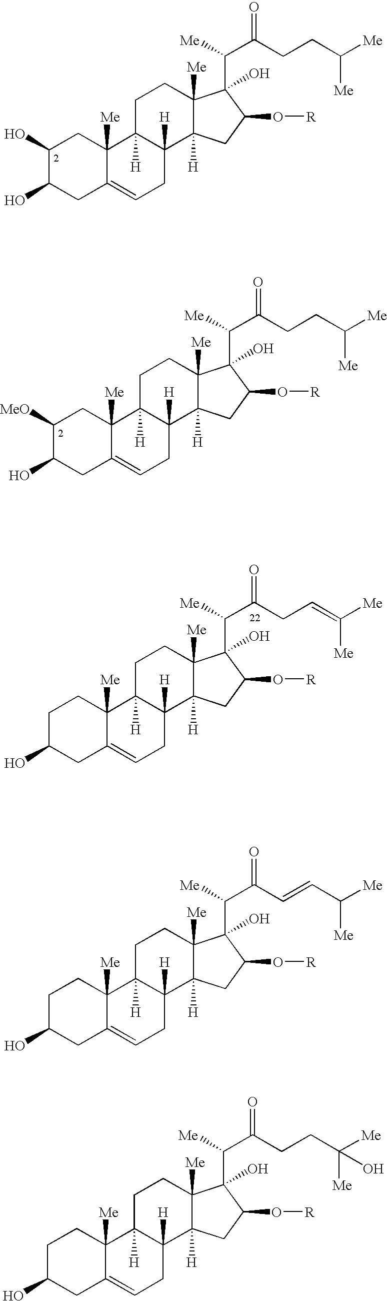 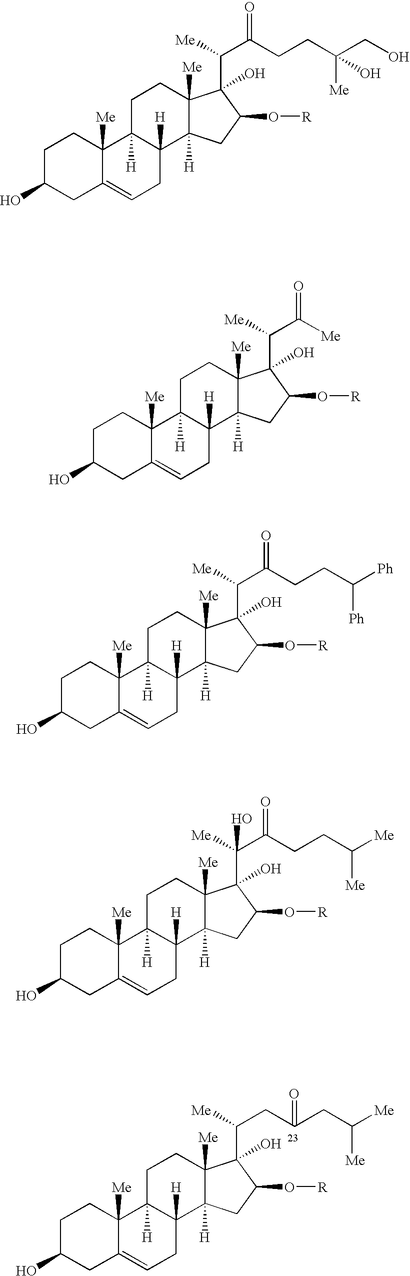 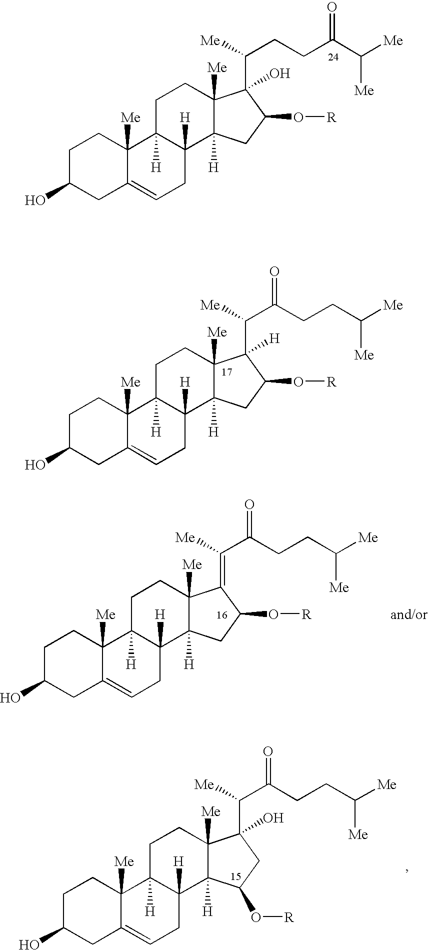
wherein R=a monosacharride, a disachharide, a trisaccharide or a polysaccharide. Pharmaceutical compositions comprising one or more of these compounds are useful in the treatment methods of the invention.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Induction of apoptosis by Orsaponin (OSW-1) in human leukemia cells. HL-60 cells in exponentially growing phase were treated with 0.5 nM OSW-1 for the indicated times. Drug induced apoptosis was analyzed by the annexin V assay as shown in FIG. 1A and by the DNA fragmentation assay as shown in FIG. 1B. In FIG. 1A, the early and late stages of apoptotic cells appear in the low-right window and upper-right window, respectively.

FIG. 2. Effect of Orsaponin (OSW-1) on cell growth in two human cancer cell lines including a leukemia and a lymphoma cell line. HL-60 (leukemia) and Raji cells (lymphoma) in exponentially growing phase were treated with the indicated concentrations of OSW-1 for 72 hours. Cell growth inhibition was measured by MTT assay.

FIG. 3. Effect of Orsaponin (OSW-1) on cell growth in human pancreatic cancer cells. Human pancreatic cancer AsPC-1 cells were treated with the indicated concentrations of OSW-1 for 72 h or 96 h. Cell growth inhibition was measured by MTT assay.

FIG. 4. The anticancer activity of Orsaponin (OSW-1) is not affected by NFκB expression in pancreatic cancer cells. Human pancreatic cancer cells AsPC-1 with constitutive activation of NFκB or inactivation of NFκB by dominant negative IκBα (AsPC-1/IκBα-ND) were treated with the indicated concentrations of OSW-1 for 72 h. Cell growth inhibition was measured by MTT assay.

FIG. 5. Effect of Orsaponin (OSW-1) on cell growth in human ovarian cancer cells (SKOV3). Human ovarian cancer SKOV3 cells were treated with the indicated concentrations of OSW-1 for 72 h. Cell growth inhibition was measured by the MTT assay. The IC₅₀ value is approximately 0.2 nM under the experimental conditions.

FIG. 6. Effect of OSW-1 on cell survival in human colon carcinoma cells. Human colon cancer cells with wild-type p53 (HCT116 p53+/+) and p53-null (HCT116 p53−/−) were treated with the indicated concentrations of OSW-1, and cell survival was measured by colony formation assay.

FIG. 7. Effect of Orsaponin (OSW-1) on cell survival in primary human leukemia cells isolated from patients with chronic lymphocytic leukemia (CLL). Freshly isolated primary CLL cells were incubated with the indicated concentrations of OSW-1 for 72 hours in vitro. Cell viability was measured by MTT assay. The IC₅₀ value is 0.15±0.26 nM (n=23 patients). Data from representative experiments with CLL cells from three different patients are shown. A total of 23 CLL blood samples were tested for sensitivity to OSW-1 in the same fashion. The mean IC₅₀ value of the 23 samples is 0.15±0.26 nM.

FIG. 8. Effect of Orsaponin (OSW-1) on cell survival in primary normal lymphocytes isolated from healthy donors. Freshly isolated normal lymphocytes were incubated with the indicated concentrations of OSW-1 for 72 hours in vitro. Cell viability was measured by MTT assay. The IC₅₀ value estimated to be 4 nM and 3 nM in case #1 and #2, respectively.

FIG. 9. Mitochondrial respiration plays an important role in the cytotoxic action of Orsaponin (OSW-1). The parental HL-60 line and its mutant with mitochondrial respiration defect, clone C6F, were incubated with 0.5 nM OSW-1 for the indicated times. Apoptosis and change in cell cycle distribution were assayed by flow cytometry analysis.

FIG. 10. Effect of Orsaponin (OSW-1) on Mitochondrial transmembrane potential HL-60 cells. The parental HL-60 line and its mutant with mitochondrial respiration defect, clone C6F, were incubated with 0.5 nM OSW-1 for the indicated times. Change in mitochondrial transmembrane potential was measured by cytometry analysis, using rhodamine-123 as a potential-sensitive fluorescent dye.

FIG. 11. Effect of orsaponin (OSW-1) on mitochondrial transmembrane potential ML-1 cells. The parental ML-1 line and its mutant with mitochondrial respiration defect, clone C19, were incubated with 1 nM OSW-1 for the indicated times. Change in mitochondrial transmembrane potential was measured by cytometry analysis, using rhodamine-123 as a potential-sensitive fluorescent probe.

FIG. 12. Nude mice were inoculated with human ovarian cancer SKOV3 cells (2×10⁶/mouse, i.p., 10 mice/group). Drug treatment started on day 6 after tumor inoculation. OSW-1 was given by i.p. injection, 10 μg/kg/day, 5 days/week for two weeks.

FIG. 13. Structure of 17-deoxyorsaponin.

FIG. 14. Comparison of anticancer activities of Orsaponin and 17-deoxyorsaponin in human leukemia cells.

FIG. 15A-15B. Effect of Orsaponin and 17-deoxyorsaponin in pancreatic cancer cells. FIG. 15A-AsPC-1 cells. FIG. 15B-Panco-2 cells.

FIG. 16. Anticancer activity of 17-deoxyorsaponin in human colon cancer cells.

FIG. 17. Effect of 17-deoxyorsaponin in human ovarian cancer cells.

FIG. 18. Effect of 17-deoxyorsaponin in human acute myeloid leukemia cells.

FIG. 19. Cytotoxic activity of 17-deoxyorsaponin in primary human leukemia cells isolated from patients with chronic lymphocytic leukemia (CLL).

FIG. 20. Comparison of cytotoxic effect of Orsaponin and 17-deoxyorsaponin in primary human leukemia cells isolated from patients with chronic lymphocytic leukemia.

FIG. 21. Antiproliferative effect of Orsaponin in human malignant glioma cells and normal human astrocytes.

FIG. 22. Selective anticancer activity of 17-deoxyorsaponin in human malignant glioma cells in comparison with normal human astrocytes.

### DESCRIPTION OF ILLUSTRATIVE EMBODIMENTS

Pancreatic cancers, CLL, colon cancers, and some ovarian cancers are associated with poor patient prognosis and a high incidence of mortality. Therefore, these cancers pose a challenge as they are generally resistant to currently existing treatment modalities.

The inventors have found that orsaponin OSW-1 induces cytotoxicity, induces apoptosis, and kills cancer cells of pancreatic, ovarian, colon, and CLL origins at an IC₅₀ of less than 1 nM. These results were observed in vitro in human cancer cell lines of pancreatic cancer, ovarian cancer, colon cancer, and leukemias as well as from cells isolated from human patients with CLL. The results were also observed in vivo in mouse models of ovarian cancer where animal survival was improved, and tumor volume reductions were observed

The present invention therefore provides methods for the treatment of cancers, especially, pancreatic cancers, colon cancers, ovarian cancers, and CLL, using compositions comprising orsaponin OSW-1 as well as its derivatives. It is also contemplated that other forms of leukemia, such as acute lymphocytic leukemia (ALL), acute myelogenous leukemia (AML), and chronic myelogenous leukemia (CML), as well as solid tumors such as lung cancer, breast cancer, liver cancer, prostate cancer, uterine cancers, colon cancer, rectal cancer, bone cancer, and brain cancers may be treated using orsaponins.

The orsaponin compositions of the invention can be administered by different modes to a cancer patient, such that these patients are conferred a therapeutic benefit as a result of the treatment. The term “therapeutic benefit” used herein refers to anything that promotes or enhances the well-being of the patient with respect to the medical treatment of the patient's cancer. A list of nonexhaustive examples of this includes extension of the patient's life by any period of time; decrease or delay in the neoplastic development of the disease; decrease in hyperproliferation; reduction in tumor growth; delay or prevention of metastases; reduction in the proliferation rate of a cancer cell or tumor cell; cancer cell cytotoxicity, induction of apoptosis in any cancer cell; a decrease in cancer cell growth; and/or a decrease in pain to the patient that can be attributed to the patient's condition.

The result of this treatment can be the induction of apoptosis, inhibition of cell division, inhibition of metastatic potential, reduction of tumor burden, increased sensitivity to chemotherapy or radiotherapy, killing of a cancer cell, inhibition of the growth of a cancer cell, induction of tumor necrosis, and induction of tumor regression of a pancreatic cancer cell, a CLL cell, a colon cancer cell, or an ovarian cancer cell.

I. OSW-1

OSW-1 (depicted by 1 in the structure below), is a natural saponin, with anticancer properties, and its four natural analogs (depicted by 2-5 in the structure below) have been isolated from the bulbs of Ornithogalum saundersiae, a perennial grown in southern Africa where it is cultivated as a cut flower and garden plant (Kubo et al., 1992). These saponins are members of the cholestane glycosides and their absolute structures have been determined by extensive application of spectroscopic methods. The structure of compounds 1-5 is characterized by the attachment of a disaccharide to the C-16 position of the steroid aglycone, whereas compounds 4 and 5 have another glycosyl sugar associated with the C-3 alcohol position of the steroid.

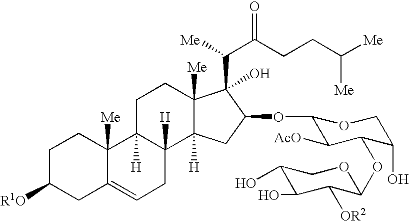

Compounds 1-5 exhibited extremely potent cytostatic activity in vitro against human promyelocytic leukemia HL-60 cells, showing IC₅₀ values ranging between 0.1 and 0.3 nM. The activity of OSW-1 (1) in this assay is much more potent than that of clinically used anticancer agents such as etoposide, adriamycin, and methotrexate (Mimaki et al., 1997). OSW-1 (1), the main constituent of the bulbs, exhibited exceptionally potent cytostatic activities against various human malignant tumor cells (Mimaki et al., 1997). Its cytostatic activities are from 10- to 100-fold more potent than some well-known anticancer agents in clinical use, such as mitomycin C, adriamycin, cisplatin, camptothecin, and even taxol, but it has significantly lower toxicity (IC₅₀ 1500 nM) to normal human pulmonary cells (Mimaki et al., 1997). The surprising similarity of the cytotoxicity profile of OSW-1 to that of cephalostatins, (Pettit et al., 1988; LaCour et al., 1998) one of the most active anticancer agents tested by NIH, with correlation coefficient of 0.60-0.83, suggests they might have the same mechanism of action (Guo and Fuchs, 1998). It has been speculated by Fuchs that the C22-oxonium ions might be the active intermediate for the potent anticancer activity of OSW-1 (1) and cephalostatins (Guo et al., 1999).

II. Methods of Synthesis of OSW-1

Several methods for the synthesis of OSW-1 have been described (Deng et al., 1999; Yu and Jin, 2001; Morzycki et al., 2002, the entire contents of which are incorporated herein by reference).

One of the inventors of the present application, Dr. Zhendong Jin, has successfully synthesized OSW-1 and details of the synthetic methods can be found in co-pending U.S. Patent Application Publication No. US 2003/0069214 A1 filed Aug. 6, 2002, which has a priority date of Aug. 7, 2001, and in Yu W and Jin Z, (2001), the entire contents of which are incorporated herein by reference. Accordingly, OSW-1 can be synthesized, from commercially available 5-androsten-3β,-ol-17-one 79 in ten operations with a 28% overall yield (see details in the schemes and description set forth below). The key steps in the total synthesis include a highly regio- and stereoselective selenium dioxide-mediated allylic oxidation of 80 and a highly stereoselective 1,4-addition of α-alkoxy vinyl cuprates 68 to steroid 17(20)-en-16-one 12E to introduce the steroid side chain.

General Methods. All moisture-sensitive reactions were performed in flame-dried glassware under a positive pressure of nitrogen or argon. All reactions were monitored by thin layer chromatography (TLC). Products were isolated or purified by flash column chromatography (silica gel, 230-400 mesh, purchased from Scientific Adsorbents Incorporated), preparative TLC, or distillation under reduced pressure. Optical rotations were measured with Jasco P-1020 polarimeter. ¹H-NMR and ¹³C-NMR spectra were recorded with a Bruker WM360 (360 MHz) instrument or Bruker DRX400 (400 MHz) instrument. 2D NMR spectra were recorded with a Bruker DRX400 (400 MHz) instrument. The deuterated solvents for NMR spectroscopy were chloroform-d₁ (CDCl₃), benzene-d₆ (C₆D₆), pyridine-d₅ (C₅DSN), or water-d₂ (D20), and are reported in parts per million (ppm) with residual protonated solvent peak or solvent ¹³C-NMR peak as internal standard (CDCl₃: 7.26 ppm for ¹H-NMR and 77.0 ppm for ¹³C-NMR; C₆D₆: 7.15 ppm for ¹H-NMR and 128.0 ppm for ¹³C-NMR; C₅D₅N: 7.58 (middle peak) for ¹H-NMR and 135.91 (middle peak) for ¹³C-NMR). When D₂O was the solvent, DDS (sodium 2,2-dimethyl-2-silapentane-5-sulfonate) was used as a reference. When peak multiplicity is reported, the following abbreviations are used: s (singlet), bs (broad singlet) d (doublet), t (triplet), q (quartet), hept (heptet), m (multiplet), b (broad), ABq (AB quartet). Mass spectra were provided by Mass Spectrometry Service Laboratory of Department of Chemistry, University of Minnesota, Mass. Spectrometry Resource of the Department of Chemistry, Wash. University at St. Louis, and the University of Iowa High Resolution Mass Spectrometry Facility.

A. Compound 19

A solution of dry 1,2,3,4-O-tetraacetyl-L-arabinose 17 (18.3 g, 57.5 mmol) in dry CH₂Cl₂ (200 mL) was cooled to −78° C. (dry-ice-acetone bath). Thiophenol (6.5 mL, 63.3 mmol) was added followed by the addition of SnCl₄ (1M in CH₂Cl₂, 17.3 mL, 17.3 mmol). The reaction solution was stirred at −78° C. for 6 hours until the starting material disappeared on TLC. Saturated aqueous NaHCO₃ (100 mL) was added. The organic layer was separated and the water layer was extracted with CH₂Cl₂ (30 mL) three times. The combined organic layer was washed with brine and dried over anhydrous Na₂SO₄. The solvent was removed and the product was purified by silica gel column chromatography to afford thiophenyl-2,3,4-O-triacetyl-L-arabinose 18 (16.95 g, 80%). Compound 18 (8.88 g, 24.1 mmol) was dissolved in MeOH (120 mL) followed by the addition of sodium methoxide (65 mg, 1.2 mmol). The reaction solution was stirred at 25° C. for six hours, then saturated aqueous NH₄Cl was added. The organic layer was separated and the water layer was extracted with CH₂Cl₂ (30 mL) three times. The combined organic layer was washed with brine and dried over anhydrous Na₂SO₄. The solvent was removed and the product was purified by silica gel column chromatography to afford 19 (5.53 g, 22.9 mmol, 95%) as pale yellow syrup; ¹H NMR (400 MHz, D₂O): δ 7.57 (m, 2H), 7.40 (m, 3H), 4.72 (d, J=8.3 Hz, 1H), 4.00 (s, 1H), 3.93 (dd, J=12.8, 2.3 Hz, 1H), 3.67 (m, 3H); ¹³C NMR (100 MHz, D₂O, DSS as reference): δ 135.0, 134.4, 132.2, 130.8, 91.2, 76.1, 72.5, 72.0, 71.2; HRMS (FAB) m/z 265.0517 (M+Na)⁺, calculated for C₁₁H₁₄O₁₄SNa: 265.0511; [α]_{D}²³−12.6 (c 0.4, CHCl₃).

B. Compounds 20,21

To a solution of 19 (4.85 g, 20.0 mmol) in CH₂Cl₂ (100 mL) was added 2,2-dimethoxypropane (2.96 mL, 24.0 mmol) and CSA (30 mg) at 25° C. The reaction solution was stirred at 25° C. for 12 hours and was quenched with saturated aqueous NaHCO₃ (30 mL). The organic layer was separated and the water layer was extracted with CH₂Cl₂ (20 mL) three times. The combined organic layer was washed with brine and dried over anhydrous Na₂SO₄. The solvent was removed and the reaction mixture was carefully dried and then was dissolved in CH₂Cl₂ (100 mL). Triethylamine (4.2 mL, 30 mmol), acetyl anhydride (2.26 mL, 24.0 mmol), and DMAP (50 mg) were added. The reaction was stirred at 25° C. for two hours, then was quenched with saturated aqueous NaHCO₃ (30 mL). The organic layer was separated and the water layer was extracted with 30 mL CH₂Cl₂ three times. The combined organic layer was washed with brine and dried over anhydrous Na₂SO₄. The solvent was removed to give compound 20 as pale yellow syrup. Compound 20 was dissolved in MeOH (100 mL) followed by the addition of Amberlite IR-118H (4.0 g). The reaction was stirred at 25° C. for 12 hours and the solid was filtered. The solvent was removed and the product was purified by silica gel chromatography to afford 21 (5.1 g, 90% for two steps from 19) as pale yellow syrup.

20: ¹H NMR (360 MHz, CDCl₃): δ 7.50˜7.47 (2H), 7.30˜7.26 (3H), 5.16 (dd, J=6.6, 5.4 Hz, 1H), 4.86 (d, J=6.6 Hz, 1H), 4.33-4.24 (2H), 4.18 (t, J=5.1 Hz, 1H), 3.80 (dd, J=11.3, 3.5 Hz, 1H), 2.13 (s, 3H), 1.56 (s, 3H), 1.35 (s, 3H); ¹³C NMR (90 MHz, CDCl₃): δ 169.2, 133.8, 131.4, 128.7, 127.3, 110.2, 85.5, 75.3, 71.9, 70.9, 63.9, 27.4, 26.0, 20.7; HRMS (FAB) m/z 347.0928 (M+Na)⁺, calculated for C₁₆H₂₀O₅SNa: 347.0929; [α]_{D}²⁴−15.5 (c 0.9, CHCl₃).

21: ¹H NMR (360 MHz, CDCl₃): δ 7.50˜7.48 (2H), 7.33˜7.26 (3H), 5.07 (t, J=7.9 Hz, 1H), 4.75 (d, J=7.6 Hz, 1H), 4.15 (dd, J=12.2, 4.0 Hz, 1H), 4.01 (bs, 1H), 4.78 (bs, 1H), 3.59 (dd, J=12.2, 1.8 Hz, 1H), 3.40 (d, J=6.8 Hz, 1H), 3.09 (d, J=5.8 Hz, 1H), 2.14 (s, 3H); ¹³C NMR (90 MHz, CDCl₃): δ 170.9, 133.6, 132.0, 128.9, 127.7, 86.3, 71.9, 67.9, 67.5, 21.0; HRMS (FAB) m/z 323.0370 (M+K)⁺, calculated for C₁₃H₁₆O₅SK: 323.0370; [α]_{D}²³+14.6 (c 1.7, CHCl₃).

C. Compound 15

A solution of 21 (2.22 g, 7.81 mmol) and 2,6-lutidine (1.80 mL, 15.6 mmol) in anhydrous CH₂Cl₂ (260 mL) was cooled to −60° C. Triethylsilyl triflate (2.10 mL, 9.37 mmol) was added dropwise. The reaction was stirred at −60° C. for one hour and then at −70° C. for another hour. The reaction was quenched with saturated aqueous NaHCO₃ (50 mL) at −70° C. and was allowed to warm up to 25° C. The organic layer was separated and the water layer was extracted with CH₂Cl₂ (30 mL) three times. The combined organic layer was washed with brine and dried over anhydrous Na₂SO₄. The solvent was removed and the product was isolated by silica gel column chromatography to afford 15 (2.80 g. 90%). ¹H NMR (360 MHz, CDCl₃): δ 7.62 (d, 8.3 Hz, 2H), 7.05˜6.93 (3H), 5.51 (t, J=5.76 Hz, 1H), 5.03 (d, J=5.4 Hz, 1H), 4.07 (dd, J=11.9, 6.1 Hz, 1H), 3.83 (m, 1H), 3.67 (m, 1H), 2.88 (d, J=11.9 Hz, 1H), 2.54 (d, J=6.1 Hz, 1H), 1.71 (s, 3H), 0.86 (t, J=8.06 Hz, 2H), 0.40 (q, J=8.06 Hz, 3H); ¹³C NMR (90 MHz, CDCl₃): δ 169.5, 135.5, 132.4, 129, 127.5, 86.0, 72.5, 71.5, 68.5, 65.0, 20.5, 6.8, 5.1; HRMS (FAB) m/z 421.1460 (M+Na)⁺, calculated for C₁₉H₃₀O₅SSiNa: 421.1481; [α]_{D}²³−33.6 (c 0.7, CHCl₃).

D. Compound 23

Dry 1,2,3,4-O-tetraacetyl-D-xylose 22 (33.40 g, 104.9 mmol) was dissolved in dry CH₂Cl₂ and was cooled to 0° C. HBr (30% in AcOH, 80 mL) was added slowly by funnel. The reaction was stirred at 0° C. for one hour and at 25° C. for three hours. The reaction solution was washed with water (50 mL) first, then the organic layer was poured into cold saturated aqueous NaHCO₃ with stirring. The organic layer was separated and the water layer was extracted with CH₂Cl₂ (50 mL) three times. The combined organic layer was washed with brine and dried over anhydrous Na₂SO₄. The solvent was removed to afford 23 (33.1 g, 93%).

E. Compound 24

Carefully dried 23 (8.50 g, 23.9 mmol) was dissolved in dry nitromethane (30 mL, distilled over CaH₂) and thioethanol (3.55 mL, 47.9 mmol) and 2,6-lutidine (4.2 mL, 35.9 mmol, distilled over CaH₂) was added. The reaction solution was stirred under nitrogen at 25° C. for 12 hours. The reaction was quenched with saturated aqueous NaHCO₃. The organic layer was separated and the water layer was extracted with CH₂Cl₂ (30 mL) three times. The combined organic layer was washed with brine and dried over anhydrous Na₂SO₄. The solvent was removed and the product was isolated by silica gel column chromatography to afford 24 (6.29 g, 82%). ¹H NMR (360 MHz, CDCl₃): δ 5.55 (d, J=4.7 Hz, 1H), 5.20 (t, J=2.9 Hz, 1H), 4.86 (m, 1H), 4.35 (m, 1H), 3.91 (dd, J=12.2, 6.5 Hz, 1H), 3.59 (dd, J=12.2, 8.3 Hz, 1H), 2.59 (q, J=7.2 Hz, 2H), 2.08 (s, 3H), 2.05 (s, 3H), 1.92 (s, 3H), 1.22 (t, J=7.6 Hz, 3H); ¹³C NMR (90 MHz, CDCl₃): δ 169.7, 169.1, 116.5, 96.7, 73.5, 68.8, 67.8, 58.8, 27.6, 24.6, 20.7, 14.9; HRMS (FAB) m/z 321.0993 (M+H)⁺, calculated for C₁₃H₂₁O₇S: 321.1008; [α]_{D}²⁴+11.3 (c 1.0, CHCl₃).

F. Compound 25

To a solution of 24 (3.679 g, 11.48 mmol) in MeOH (60 mL) was added sodium methoxide (30 mg, 0.57 mmol). The reaction solution was stirred at 25° C. for 3 hours and MeOH was removed to afford 25 (2.750 g) which was used in the next step without further purification. ¹H NMR (360 MHz, CDCl₃): δ 5.51 (d, J=4.0 Hz, 1H), 4.24 (t, J=3.6 Hz, 1H), 4.02 (t, J=4.0 Hz, 1H), 3.89 (q. J=14, 6.1 Hz, 1H), 3.65 (dd, J=12.6, 3.7 Hz, 1H), 2.62 (q, J=7.6 Hz, 2H), 1.92 (s, 3H), 1.22 (t, J=7.2 Hz, 3H); ¹³C NMR (90 MHz, CDCl₃): δ 116.6, 97.1, 70.7, 68.0, 63.5, 28.7, 24.7, 14.9; HRMS (FAB) m/z 237.0788 (M+H)⁺, calculated for C₉H₁₇O₅S: 237.0797; [α]D²³+35.6 (c 0.8, CHCl₃).

G. Compound 26

To a solution of 25 (2.714 g, 11.48 mmol) in dry THF (100 mL) was added NaH (60% in mineral oil, 1.45 g, 36 mmol) at 0° C. The reaction solution was stirred at 25. ° C. for 10 min and p-methoxybenzyl chloride (3.3 mL, 24.3 mmol) and tetrabutylammonium iodide (100 mg, 0.27 mmol) were added. The reaction was stirred under reflux for 4 hours. After it was cooled down, the reaction was quenched with saturated aqueous NaHCO₃. The organic layer was separated and the water layer was extracted with ethyl acetate (30 mL) three times. The combined organic layer was washed with brine and dried over anhydrous Na₂SO₄. The solvent was removed and the product was isolated by silica gel column chromatography to afford 26 (5.14 g, 94% from 24). ¹H NMR (360 MHz, CDCl₃): δ 7.28 (d, J=7.6 Hz, 2H), 7.22 (d, J=8.6 Hz, 2H), 6.89 (d, J=7.6 Hz, 2H), 6.87 (d, J=8.3 Hz, 2H), 5.62 (d, J=5.0 Hz, 1H), 4.61 (d, J=11.5 Hz, 1H), 4.53 (d, J=11.9 Hz, 1H), 4.49 (d, J=13.3 Hz, 1H), 4.45 (dd, J=5.0, 2.5 Hz, 1H), 3.86 (dd, J=3.6, 2.9 Hz, 1H), 3.81 (s, 3H), 3.80 (s, 3H), 3.77 (d, J=5.8 Hz, 1H), 3.65 (m, 1H), 3.57 (dd, J=10.8, 9.4 Hz, 1H), 2.63 (q, J=7.2 Hz, 2H), 1.93 (s, 3H), 1.27 (t, J=7.2 Hz, 3H); ¹³C NMR (90 MHz, CDCl₃): δ 159.4, 159.3, 129.9, 129.5, 129.3, 115.6, 113.8, 97.7, 77.3, 75.3, 74.2, 71.6, 71.4, 60.1, 55.2, 27.9, 24.7, 15.1; HRMS (FAB) m/z499.1759 (M+Na)⁺, calculated for C₂₅H₃₂O₇SNa: 499.1767; [α]_{D}²³+16.8 (c 1.1, CHCl₃).

H. Compounds 27, 28

To a solution of carefully dried 26 (3.93 g, 8.25 mmol) in dry CH₂Cl₂ (20 mL) was added zinc chloride (1M in ether, 0.5 mL, 0.5 mmol) at −60° C. The reaction was stirred at −60° C. for 30 min and was warmed up to 0° C. in 30 min. The reaction was quenched with saturated aqueous NaHCO₃. The organic layer was separated and the water layer was extracted with CH₂Cl₂ (15 mL) three times. The combined organic layer was washed with brine and dried over anhydrous Na₂SO₄. The solvent was removed to afford crude product 27. The crude 27 was dissolved in MeOH (40 mL) and sodium methoxide (22 mg, 0.4 mmol) was added. The reaction was stirred at 25° C. for 4 hours. The solvent was removed and the product was isolated by silica gel column chromatography to afford 28 (3.4 g, 95% from 26).

27: ¹H NMR (360 MHz, CDCl₃): δ 7.24 (d, J=7.2 Hz, 2H), 7.21 (d, J=7.2 Hz, 2H), 6.86 (d, J=7.2 Hz, 4H), 4.92 (t, J=8.6 Hz, 1H), 4.74 (d, J=11.2 Hz, 1H), 4.62 (d, J=11.2 Hz, 2H), 4.53 (d, J=11.2 Hz, 1H), 4.36 (d, J=9.0 Hz, 1H), 4.03 (dd, J=11.9, 5.0 Hz, 1H), 3.80 (s, 3H), 3.62˜3.55 (m, 2H), 3.24 (dd, J=9.0, 1.2 Hz, 1H), 2.65 (m, 2H), 2.01 (s, 3H), 1.23 (t, J=7.6 Hz, 3H); ¹³C NMR (90 MHz, CDCl₃): δ 169.6, 159.3, 159.2, 130.4, 130.0, 129.4, 113.8, 113.7, 83.8, 81.5, 74.2, 72.8, 71.2, 67.2, 55.2, 23.9, 20.9, 14.8; HRMS (FAB) m/z 499.1772 (M+Na)⁺, calculated for C₂₅H₃₂O₇SNa: 499.1767; [α]_{D}²⁴−8.0 (c 0.8, CHCl₃).

28: ¹H NMR (360 MHz, CDCl₃): δ 7.30 (d, J=8.6 Hz, 2H), 7.24 (d, J=8.6 Hz, 2H), 6.88 (d, J=8.6 Hz, 2H), 6.87 (d, J=8.6 Hz, 2H), 4.76 (d, J=11.5 Hz, 1H), 4.72 (d. J=11.5 Hz, 1H), 4.61 (d, J=11.2 Hz, 1H), 4.55 (d, J=11.2 Hz, 1H), 4.52 (d, J=7.2 Hz, 1H), 4.11 (dd, J=11.5, 3.2 Hz, 1H), 3.79 (s, 6H), 3.55 (m, 2H), 3.37 (dd, J=11.5, 7.6 Hz, 1H), 3.09 (d, J=4.7 Hz, 1H), 2.68 (m, 2H), 1.29 (t, J=7.6 Hz, 3H); ¹³C NMR (90 MHz, CDCl₃): δ 159.2, 159.1, 130.3, 129.7, 129.3, 113.7, 86.2, 80.4, 77.2, 76.0, 73.7, 72.2, 71.5, 64.9, 55.0, 24.8, 15.1; HRMS (FAB) m/z 435.1849 (M+H)⁺, calcd for C₂₃H₃₁O₆S: 435.1841; [α]_{D}²³−56.7 (c 0.6, CHCl₃).

I. Compound 29

To a solution of 28 (3.2 g, 7.36 mmol) and triethylamine (1.54 mL, 11.04 mmol) in CH₂Cl₂ (20 mL) was added 4-methoxybenzoyl chloride (1.33 mL, 9.57 mmol) and DMAP (45 mg, 0.37 mmol). The reaction was stirred at 25° C. for 48 hours. Small amount of reaction solution was taken out and quenched with saturated aqueous NaHCO₃, and then diluted with 1 mL CH₂Cl₂. The organic layer was separated and the solvent was removed. The crude mixture was checked with ¹H-NMR. When the NMR signal of 28 disappeared, the reaction was quenched with saturated aqueous NaHCO₃. The organic layer was separated and the water layer was extracted with CH₂Cl₂ (30 mL) three times. The combined organic layer was washed with brine and dried over anhydrous Na₂SO₄. The solvent was removed and the product was isolated by silica gel column chromatography to afford 29 (4.06 g, 97%). ¹H NMR (360 MHz, CDCl₃): δ 7.97 (d. J=8.6 Hz, 2H), 7.25 (d, J=8.1 Hz, 2H), 7.10 (d, J=8.6 Hz, 2H), 6.90 (d, J=8.6 Hz, 2H), 6.87 (d, J=8.1 Hz, 2H), 6.69 (d, J=8.6 Hz, 2H), 5.20 (t, J=8.3 Hz, 1H), 4.73˜4.55 (m, 5H), 4.11 (dd, J=11.5, 4.3 Hz, 1H), 3.84 (s, 3H), 3.79 (s, 3H), 3.70 (s, 3H), 3.35(dd, J=11.5, 9 Hz, 1H), 2.67 (m, 2H), 1.22 (t, J=7.2H, 3H). ¹³C NMR (90 MHz, CDCl₃): δ 164.7, 163.3, 159.1, 158.9, 131.7, 129.9, 129.4, 129.3, 122.1, 113.6, 113.4, 83.8. 80.9, 77.2, 76.8, 73.9, 72.6, 71.2, 66.7, 55.2, 55.0, 54.8, 24.0, 14.7; HRMS (FAB) m/z 591.2044 (M+Na)⁺, calculated for C₃₁H₃₆O₈SNa: 591.2029; [α]_{D}²⁵+33.0 (c 1.3, CHCl₃).

J. Compound 16

To a solution of 29 (2.31 g, 4.06 mmol) in CH₂Cl₂ (25 mL) and water (3 mL) was added NBS (0.795 g, 4.46 mmol) in one portion. After the reaction was stirred at 25° C. for 1 hour, saturated aqueous Na₂SO₃ was added. The organic layer was separated and the water layer was extracted with CH₂Cl₂ (15 mL) three times. The combined organic layer was washed with brine and dried with anhydrous Na₂SO₄. The solvent was removed and the product was isolated by silica gel column chromatography to afford 3,4-di-O-(4-methoxybenzyl)-2-O-(4-methoxybezoyl)-α/β-D-xylopyranose (1.87 g, 88%). Carefully dried 3,4-di-O-(4-methoxybenzyl)-2-O-(4-methoxybenzoyl)-α/β-D-xylopyranose (413.3 mg, 0.788 mmol) was dissolved in dry CH₂Cl₂ (4 mL) and trichloroacetonitrile (0.4 mL, 3.94 mmol) and DBU (1 drop) were added. The reaction was stirred at 25° C. for 12 hours. The solvent was removed and the product was isolated by silica gel column chromatography (deactivated by 3% Et₃N in hexane) to afforded 16 (555 mg, 95%) which was used immediately without identification.

K Compound 30

A solution of carefully dried 15 (339 mg, 0.718 mmol), 16 (941 mg, 1.407 mmol) and 4 Å MS powder (150 mg) in dry CH₂Cl₂ (3 mL) was stirred at 25° C. for 15 min, then was cooled to −78° C. BF₃.Et₂O (0.1 M in CH₂Cl₂, 0.7 mL, 0.07 mmol) was added. The reaction was gradually warmed up to −40° C. and was stirred at −40° C. for 2 hours, then at −20° C. for another 2 hours. Et₃N (0.1 mL) was added and the reaction solution was filtered. The solvent was removed and the product was isolated by silica gel column chromatography to afford 30 (602 mg, 93%) as pale yellow syrup. ¹H NMR (360 MHz, C₆D₆): δ 8.17 (d, J=9.0 Hz, 2H), 7.50 (d, J=7.6 Hz, 2H), 7.26 (d, J=8.6 Hz, 2H), 6.99 (t, J=7.6 Hz, 2H), 6.92 (t, J=6.8 Hz, 1H), 6.78 (d, J=8.6 Hz, 2H), 6.67 (d, J=8.3 Hz, 2H), 6.65 (d, J=9 Hz, 2H), 5.73 (t, J=7.2 Hz, 1H), 5.63 (t, J=6.5 Hz, 1H), 4.93 (d, J=6.1 Hz, 1H), 4.86 (d, J=11.2 Hz, 1H), 4.81 (d, J=11.2 Hz, 1H), 4.45 (d, J=11.2, 1H), 4.31 (d, J=11.2 Hz, 1H), 4.13˜4.05 (m, 2H), 3.90 (t, J=7.6 Hz, 1H), 3.67 (m, 1H), 3.30 (s, 3H), 3.23 (s, 3H), 3.15 (s, 3H), 1.66 (s, 3H), 1.04 (t, J=7.9 Hz, 9H), 0.66 (m, 6H); ¹³C NMR (90 MHz, C₆D₆): δ 168.6, 164.9, 163.6, 159.8, 159.6, 132.4, 132.1, 130.9, 129.9, 129.6, 128.8, 127.2, 123.5, 114.2, 113.8, 86.4, 79.9, 77.7, 74.0, 72.6, 72.4, 70.7, 70.7, 63.4, 54.8, 54.7, 54.6, 20.6, 7.1, 5.2; HRMS (FAB) m/z 927.3412 (M+Na)⁺, calculated for C₄₇H₆₈O₁₅Na: 927.3422; [α]_{D}²⁴−2.5 (c 1.3, CHCl₃).

L. Compound 10

To a solution of compound 30 (129 mg, 0.143 mmol) in CH₂Cl₂—H₂O (10:1, 2 mL) was added N-bromosuccinimide (NBS) (31 mg, 0.172 mmol). The reaction was stirred at 25° C. for two hours and then was quenched with saturated aqueous Na₂SO₃. The organic layer was separated and the water layer was extracted with CH₂Cl₂ (3 mL) three times. The combined organic layer was washed with brine and dried over anhydrous Na₂SO₄. The solvent was removed and the product was isolated by silica gel column chromatography to afford 2-O-acetyl-3-O-(3,4-di-O-(4-methoxybenzyl)-2-O-(4-methoxybenzoyl)-β-D-xylopyranosyl)-4-O-(triethylsilyl)-β-L-arabinopyranose (93.4 mg, 81%). To a solution of 2-O-acetyl-3-O-(3,4-di-O-(4-methoxybenzyl)-2-O-(4-methoxybenzoyl)-β-D-xylopyranosyl)-4-O-(triethylsilyl)-β-L-arabinopyranose (93.4 mg) in dry CH₂Cl₂ (2 mL) was added trichloroacetonitrile (0.068 mL, 0.575 mmol) and DBU (1 drop). The reaction was stirred at 25° C. for 12 hours and the solvent was removed. The product was isolated by Et₃N (3% Et₃N in hexane) deactivated silica gel column chromatography to afford compound 10 (97 mg, 88%) which was used immediately without identification.

M. Compound 12Z

Compound 12Z was prepared from the major isomer of compound 32 with the same procedure as compound 12E. Compound 32 was prepared according to literature procedure. ¹H NMR (400 MHz, CDCl₃): δ 5.72 (q, J=7.5 Hz, 1H), 5.32 (d, J=4.5 Hz, 1H), 3.49 (m, 1H), 2.08 (d, J=7.0 Hz, 3H), 1.07 (s, 3H), 0.94 (s, 3H), 0.89 (s, 9H), 0.06 (s, 6H). ¹³C NMR (125 MHz, CDCl₃): δ 208.4, 148.1, 141.7, 130.1, 120.4, 72.4, 50.1, 49.7, 42.9, 42.7, 39.4, 37.1, 36.7, 35.6, 32.0, 31.6, 30.9, 25.9, 20.6, 19.4, 19.3, 18.2, 14.0, -4.6; HRMS (FAB) m/z 429.3178 (M+H)⁺, calculated for C₂₇H₄₅O₂Si: 429.3189; [α]_{D}²⁵−184.6 (c 1.3, CHCl₃).

N. Compound 66

To a solution of 65 (4.20 g, 33.5 mmol) in dry THF (50 mL) was added n-BuLi (1.38 M, 27.4 mL, 37.8 mmol) at 0° C. The reaction was stirred at 0° C. for 20 min, then was cooled to −60° C. Isobutyl triflate (7.64 g, 37.1 mmol) in THF (10 mL) was cannulated dropwise. The reaction was allowed to gradually warm up to 25° C. in 2 hours and stirred at 25° C. for 10 hours. Then 30 mL saturated aqueous NaHCO₃ was added and THF was removed by rotary evaporator. The water layer was extracted with 30 mL hexane four times. The combined organic layer was washed with brine and dried over anhydrous Na₂SO₄. The solvent was removed and the product was isolated with 3% Et₃N-hexane deactivated silica gel column chromatography to afford 66 (5.13 g. 85%). ¹H NMR (360 MHz, CDCl₃): δ 3.94 (m, 1H), 2.01 (d, J=5.8 Hz, 2H), 1.97˜1.25 (m, 11H), 0.94 (d, J=6.1 Hz, 6H); ¹³C NMR (90 MHz, CDCl₃): δ 128, 88.8, 85.2, 36.9, 30.9, 28.7, 26.6, 25.2, 23.2, 21.9. EIMS m/z 180 (M+).

O. Compound 67

To the solution of 66 (3.31 g, 18.4 mmol) and anhydrous MeOH (0.706 mL, 17.4 mmol) in CH₂Cl₂ (50 mL) was added trimethylsilyl bromide (2.30 mL, 17.4 mmol) dropwise at −40° C. The reaction solution was stirred at −40° C. for 10 min, then gradually warmed up to room temperature. The solvent was removed to afford 67 which was used without further purification. ¹H NMR (360 MHz, CDCl₃): d 5.06 (t, J=7.5 Hz, 1H), 4.08 (m, 1H), 1.94 (dd, J=7.5, 6.9 Hz, 2H), 1.77˜1.03 (11H), 0.81 (d, J=6.7 Hz, 6H); ¹³C NMR (90 MHz, CDCl₃): δ 134.1, 117.6, 82.0, 36.7, 32.2, 28.8, 25.4, 23.2, 22.3. EIMS m/z 260 (M+).

P. Compound 74

Compound 74 was prepared from 12Z with the same procedure as compound 83. ¹H NMR (360 MHz, CDCl₃): δ 5.28 (d, J=3.6 Hz, 1H), 3.88˜3.79 (m, 4H), 3.45 (m, 1H), 2.08 (s, 3H), 1.16 (d, J=6.8 Hz, 3H), 1.00 (s, 3H), 0.86 (s, 15H), 0.84 (s, 3H), 0.03 (s, 6H); ¹³C NMR (90 MHz, CDCl₃): δ 168.9, 147.3, 141.6, 136.9, 120.8, 113.8, 72.5, 65.6, 65.3, 53.1, 50.4, 46.4, 42.8, 37.7, 37.1, 36.8, 34.4, 34.2, 32.2, 32.0, 31.9, 31.3, 30.1, 28.3, 25.9, 22.7, 22.5, 21.1, 20.5, 19.3, 18.2, 16.2, 15.3, −4.6; HRMS (FAB) m/z 637.4275 (M+Na)⁺, calculated for C₃₇H₆₂O₅SiNa: 637.4264; [α]_{D}²⁵−33.9 (c 1.7, CHCl₃).

Q. Compound 75

Compound 75 was prepared from 74 by the same procedure as 84. ¹H NMR (360 MHz, CDCl₃): δ 5.31 (d, J=5.0 Hz, 1H), 4.71 (s, 1H), 4.02˜3.95 (m, 4H), 3.49 (m, 1H), 1.12 (d, J=7.2 Hz, 3H), 1.04 (s, 3H), 0.89 (overlap, 15H), 0.82 (s, 3H), 0.06 (s, 6H); ¹³C NMR (90 MHz, CDCl₃): δ 214.1, 141.5, 120.7, 114.9, 82.8, 77.3, 77.0, 76.7, 72.4, 65.0, 64.8, 49.7, 47, 2, 44.4, 42.7, 39.8, 37.9, 37.1, 36.7, 33.2, 32.0, 31.4, 31.0, 28.0, 25.9, 22.8, 22.7, 19.8, 19.5, 18.2, 14.8, 14.4, −4.6; HRMS (FAB) m/z 611.4100 (M+Na)⁺, calculated for C₃₅H₆₀O₅SiNa: 611.4108; [α]_{D}²⁵−134.9 (c 1.2, CHCl₃).

R. Compound 76

Compound 76 was prepared from 75 by the same procedure as 85. ¹H NMR (360 MHz, C₆D₆): δ 5.39 (d. J=4.7 Hz, 1H), 3.87 (m, 1H), 3.60 (m, 1H), 3.52˜3.39 (m, 4H), 1.36 (d, J=6.8 Hz, 3H), 1.18 (s, 3H), 1,00 (s, 9H), 0.99 (s, 3H), 0.88 (d, J=6.4 Hz, 6H), 0.10 (s, 3H), 0.09 (s, 3H); ¹³C NMR (90 MHz, C₆D₆): δ 141.5, 121.6, 115.7, 87.1, 81.6, 72.9, 63.6, 63.5, 50.3, 49.6, 47.3, 43.5, 40.0, 37.7, 36.9, 35.8, 32.7, 32.5, 32.3, 31.3, 28.6, 26.1, 22.9, 22.8, 21.0, 19.6, 18.3, 14.1, 13.9, −4.3; HRMS (FAB) m/z 613.4270 (M+Na)⁺, calculated for C₃₅H₆₂O₅SiNa: 613.4264; [α]_{D}²⁶−30.1 (c 0.5, CHCl₃).

S. Compound 77

Compound 77 was prepared from 76 by the same procedure as 86. ¹H NMR (400 MHz, CDCl₃): δ 7.87 (d, J=9.0 Hz, 2H), 7.23 (d, J=8.7 Hz, 2H), 7.07 (d, J=8.7 Hz, 2H), 6.86 (d, J=9.0 Hz, 2H), 6.85 (d, J=8.7 Hz, 2H), 6.66 (d, J=8.7 Hz, 2H), 5.28 (d, J=4.8 Hz, 1H), 5.07 (m, 2H), 4.69˜4.53 (overlap, 6H), 4.17 (d, J=6.8 Hz, 1H), 4.04 (overlap, 2H), 3.85 (s, 3H), 3.80 (s, 3H), 3.71 (s, 3H), 1.59 (s, 3H), 0.98˜0.83 (overlap, 15H), 0.60 (m, 6H), 0.05 (s, 9H); ¹³C NMR (100 MHz, CDCl₃): δ 168.5, 164.5, 163.3, 159.4, 159.0, 141.4, 131.8, 131.7, 130.2, 129.5, 129.4, 122.6, 121.2, 114.5, 113.9, 113.8, 113.6, 113.4, 103.8, 101.6, 90.1, 88.1, 79.8, 77.7, 77.2, 73.9, 72.8, 72.6, 72.2, 71.1, 69.7, 66.5, 64.3, 64.0, 63.1, 56.0, 55.4, 55.3, 55.1, 50.0, 49.8, 46.5, 42.8, 39.6, 37.4, 36.5, 34.5, 32.6, 32.1, 31.9, 31.4, 30.8, 28.2, 25.9, 22.68, 22.66, 20.82, 20.77, 19.4, 18.2, 13.1, 12.7, 6.8, 4.9, −4.6; HRMS (FAB) m/z 1407.7597 (M+Na)⁺, calculated for C₇₇H₁₁₆O₁₈Si₂Na: 1407.7597; [α]_{D}²⁵−20.9 (c 0.2, CHCl₃).

T. Compound 78

Compound 78 was prepared from 77 by the same procedure as 1. ¹H NMR (400 MHz, Pyr-d₆): δ 8.30 (d, J=8.4 Hz, 2H), 7.04 (d, J=8.4 Hz, 2H), 5.90 (dd, J=9.7, 7.9 Hz, 1H), 5.70 (d, J=8.8, 8.4 Hz, 1H), 5.37 (d, J=4.4 Hz, 1H), 5.19 (d, J=7.7 Hz, 1H), 4.87 (s, 1H), 4.64 (d, J=7.6 Hz, 1H), 4.50 (broad s, 1H), 4.36˜4.27 (m, 3H), 4.20˜4.15 (m, 2H), 4.01 (dd, J=7.7, 3.8 Hz, 1H), 3.81˜3.74 (m, 3H), 3.69 (s, 3H), 3.24 (q, J=6.9 Hz, 1H), 2.80˜2.63 (m, 2H), 2.60 (d, J=7.4 Hz, 1H), 2.37 (m, 1H), 1.99 (s, 3H), 1.39 (d, J=6.7 Hz, 3H), 1.03 (s, 3H), 0.98 (s, 3H), 0.84 (d, J=6.0 Hz, 6H); ¹³C NMR (90 MHz, C₆D₆): δ 218.9, 169.6, 166.0, 164.2, 142.3, 132.8, 121.5, 114.5, 104.5, 104.1, 88.4, 87.0, 81.5, 76.8, 75.8, 72.1, 71.6, 71.4, 69.4, 67.5, 67.3, 55.8, 50.6, 49.2, 47.2, 45.4, 43.9, 42.1, 38.2, 37.2, 36.0, 33.3, 33.0, 32.8, 32.5, 32.4, 28.0, 22.9, 22.8, 21.3, 20.0, 14.9, 14.7; HRMS (FAB) m/z 895.4517 (M+Na)⁺, calculated for C₄₇H₆₈O₁₅Na: 895.4456, [α]_{D}²⁴−39.4 (c 0.4, CH₃OH).

U. Compound 32E

A solution of selenium dioxide (36.0 mg, 0.319 mmol) in CH₂Cl₂ (3 mL) was added tert-BuOOH (5M, 0.165 mL, 0.825 mmol) at 0° C. The solution was stirred at 0° C. for 15 min until the solid disappeared. 80 (264 mg, 0.638 mmol) was added in one portion. After the reaction solution was stirred at 0° C. for 5 hours, it was diluted with 5 mL CH₂Cl₂ and quenched with 6 mL aqueous Na₂SO₃. The organic layer was separated and the water layer was extracted with 5 mL CH₂Cl₂ three times. The combined organic layer was washed with brine and dried with Na₂SO₄. The solvent was removed and the product was isolated by silica gel column chromatography to afford 32E (266.3 mg. 97%) as white solid. ¹H NMR (360 MHz, CDCl₃): δ 5.78 (qd, J=6.8, 0.72 Hz, 1H), 5.31 (d, J=5.0 Hz, 1H), 4.42 (s, 1H), 3.47 (m, 1H), 1.73 (d, J=7.2 Hz, 3H), 1.0 (s, 3H), 0.88 (s, 9H), 0.87 (s, 3H), 0.04 (s, 6H); ¹³C NMR (90 MHz, CDCl₃): δ 155.3, 141.5, 120.9, 119.6, 74.3, 72.5, 52.7, 50.1, 44.1, 42.8, 37.2, 37.1, 36.6, 35.1, 32.0, 31.6, 30.8, 25.9, 21.0, 19.3, 18.2, 17.2, 13.2, −4.6; HRMS (FAB) m/z 453.3146 (M+Na)⁺, calculated for C₂₇H₄₆O₂SiNa: 453.3146; [α]_{D}²⁵−49.6 (c 0.5, CHCl₃).

V. Compound 12E

To a solution of DMSO (0.095 mL, 1.34 mmol) in dry CH₂Cl₂ (2 mL) was added oxalyl chloride (0.059 mL, 0.67 mmol) in −50° C., and the solution was stirred at −50° C. for 3 min. 32E (241.1 mg, 0.56 mmol) in CH₂Cl₂ (0.7 mL) was added. The flask was washed with CH₂Cl₂ (0.3 mL) once. The reaction was stirred at −50° C. for 45 min, then Et₃N (2.3 mL, 16.8 mmol) was added. The reaction was stirred at −50° C. for 15 min, then it was warmed up to 25° C. Water (5 mL) was added and the organic layer was separated. The water layer was extracted with 5 mL CH₂Cl₂ three times. The combined organic layer was washed with brine and dried with anhydrous Na₂SO₄. The solvent was removed and the product was isolated by silica gel column chromatography to afford 12E (229.7 mg, 96%) as white solid. ¹H NMR (400 MHz, CDCl₃): δ 6.65 (q, J=7.5 Hz, 1H), 5.27 (d, J=5.2 Hz, 1H), 3.61 (m, 1H), 1.50 (d, J=7.5 Hz, 3H), 1.04 (s, 9H), 0.87 (s, 3H), 0.75 (s, 3H), 0.12 (s, 3H), 0.11 (s, 3H). ¹³C NMR (100 MHz, CDCl₃): δ 203.6, 148.1, 141.4, 127.8, 121.0, 72.7, 50.2, 50.1, 43.4, 43.0, 37.9, 37.3, 36.9, 36.3, 32.6, 31.9, 30.7, 26.1, 20.9, 19.4, 18.3, 17.3, 12.9, −4.3; HRMS (FAB) m/z 429.3174 (M+H)⁺, calculated for C₂₇H₄₅O₂Si: 429.3189; [α]_{D}²⁵−152.7 (c 1.8, CHCl₃).

W. Compounds 81, 82, and 83

A solution of 67 (17.4 mmol) in dry ether (60 mL) and dry THF (10 mL) was cooled to −78° C., then tert-BuLi (1.71M, 20.4 mL, 34.88 mmol) was added dropwise. The reaction was stirred at −78° C. for 30 min, then was cannulated to a clear solution of CuCN (781 mg, 8.72 mmol) and LiCl (740 mg, 17.4 mmol) in THF (20 ml, CuCN and LiCl was stirred at THF for 10 min at 25° C.) at −78° C. After the solution was stirred at −78° C. for 15 min, a solution of 12E (1.22 g, 2.85 mmol) and trimethylsilyl chloride (redistilled, 1.8 mL, 14.3 mmol) in THF (5 mL) was cannulated to the cuprate solution in −78° C. The reaction solution was stirred at −78° C. for 30 min, then it was gradually warmed up to 25° C. 1 mL triethylamine was added followed by 50 mL hexane. The solution was passed through a short silica gel pad which was pretreated with 5% triethylamine-hexane. The silica gel was washed with ether (10 mL) three times. The solvent was removed to afford crude product 81.

Crude 81 was dissolved in 10 mL anhydrous benzene and the benzene was removed by rotary evaporator. After this operation was repeated three times, it was dried under vacuum for 30 min. Then the mixture was dissolved in anhydrous THF (20 mL) followed by the addition of potassium tert-butoxide (1M, 3.42 mL, 3.42 mmol) at 0° C. The reaction solution was stirred at 0° C. for 10 min, then it was cooled to −30° C. Acetyl chloride (redistilled, 0.3 mL, 4.28 mmol) was added dropwise. The reaction was stirred at −30° C. for 30 min, the was allowed to warm up to 25° C. Saturated aqueous NaHCO₃ (20 mL) and ethyl acetate (20 mL) were added. The organic layer was separated and the water layer was extracted with 10 mL ethyl acetate three times. The combined organic layer was washed with brine and dried over anhydrous Na₂SO₄. The solvent was removed and the product was isolated by silica gel column chromatography to afford 82 which was still contaminated by some impurity.

60.5 mg pure 82 was removed for NMR experiment and the rest of the product was carefully dried and dissolved in anhydrous CH₂Cl₂ (30 mL). Anhydrous ethylene glycol (0.769 mL, 13.8 mmol) and PPTS (30 mg) were added. The reaction was stirred under nitrogen at 25° C. for 3 hours until the disappearance of 82 on TLC. Triethylamine (0.2 mL) and water (20 mL) were added. The organic layer was separated and the water layer was extracted with CH₂Cl₂ (10 mL) three times. The combined organic layer was washed with brine and dried with anhydrous Na₂SO₄. The solvent was removed and the product was isolated by silica gel column chromatography to afford 83 (1.274 g, 75% from 12E) as colorless syrup.

82: ¹H NMR (360 MHz, CDCl₃): δ 5.3 (d, J=4.7 Hz, 1H), 4.99 (t, J=6.8 Hz, 1H), 3.89 (m, 1H), 3.58 (m, 1H), 3.41 (q, J=6.8 Hz, 1H), 2.25 (t, J=6.1 Hz, 2H), 1.76 (s, 3H), 1.41 (d, J=7.2 Hz, 3H), 1.07 (s, 3H), 1.04 (s, 3H), 1.02 (s, 12H), 0.93 (s, 3H), 0.114 (s, 3H), 0.109 (s, 3H). ¹³C NMR (90 MHz, CDCl₃): δ 167.8, 154.7, 147.9, 141.6, 137.6, 121.3, 110.3, 74.4, 72.9, 55.3, 50.6, 46.2, 43.5, 37.3, 37.0, 35.21, 35.19, 33.4, 33.1, 32.8, 32.6, 32.2, 31.7, 30.0, 29.3, 26.1, 24.6, 23.0, 22.8, 20.9, 20.5, 19.3, 18.4, 18.3, 18.2, −4.3; HRMS (FAB) m/z 652.4833 (M⁺), calculated for C₄₁H₆₈O₄Si: 652.4887; [α]_{D}²³−22.3 (c 0.8, CHCl₃).

83: ¹H NMR (360 MHz, CDCl₃): δ 5.30 (d, J=4.7 Hz, 1H), 3.68˜3.54 (m, 5H), 2.88 (q, J=7.2 Hz, 1H), 1.80 (s, 3H), 1.32 (d, J=7.2 Hz, 3H), 1.1 (s, 3H), 1.0 (s, 12H), 0.94 (d, J=3.6 Hz, 3H), 0.93 (d, J=6.4 Hz, 3H), 0.10 (s, 3H), 0.097 (s, 3H); ¹³C NMR (90 MHz, CDCl₃): δ 168.1, 148.7, 141.7, 137.4, 121.3, 113.9, 72.9, 66.0, 65.3, 56.0, 50.7, 46.3, 43.5, 39.3, 37.4, 37.0, 35.8, 34.9, 32.8, 32.6, 32.6, 31.7, 30.3, 28.7, 26.1, 22.9, 22.9, 20.9, 20.6, 19.4, 18.3, 17.4, 14.8, −4.3; HRMS (FAB) m/z 653.4021 (M+K)⁺, calculated for C₃₇H₆₂O₅SiK: 653.4004; [α]_{D}²⁴-52 (c 1.3, CHCl₃).

X. Compound 84

To a solution of 83 (118.9 mg, 0.193 mmol) in anhydrous THF (1 mL) was added potassium tert-butoxide (1M, 0.3 mL, 0.3 mmol) at 0° C. After the reaction was stirred at 0° C. for 10 min and cooled to −78° C., it was cannulated to a solution of Davis reagent (87 mg, 0.325 mmol) in anhydrous THF (0.5 mL) in −78° C. The flask was washed once with THF (0.3 mL). The reaction was stirred at −78° C. for 30 min, then 30 mg silica gel was added in −78° C. before it was warmed up to 25° C. The silica gel was filtered and solvent was removed. The product was isolated by silica gel chromatography to give 84 (86 mg, 76%) as white solid. ¹H NMR (360 MHz, CDCl₃): δ 5.31 (d. J=4.7 Hz, 1H), 4.73 (s, 1H), 4.07˜3.94 (m, 4H), 3.49 (m, 1H), 2.74 (q, J=7.6 Hz, 1H), 1.03 (m, 6H), 0.93 (s, 3H), 0.89 (overlap, 15H), 0.06 (s, 6H); ¹³C NMR (90 MHz, CDCl₃): δ 215.5, 141.6, 120.7, 115.4, 85.4, 72.4, 63.4, 63.2, 49.5, 46.9, 45.4, 42.7, 41.1, 37.2, 37.1, 36.7, 32.7, 32.2, 32.00, 31.95, 30.7, 30.2, 28.3, 25.9, 22.7, 22.4, 20.1, 19.4, 18.2, 15.0, 14.3, −4.3; HRMS (FAB) m/z 589.4309 (M+H)⁺, calculated for C₃₅H₆₁O₅Si: 589.4288; [α]_{D}²⁵−156.2 (c 1.2, CHCl₃).

Y. Compound 85

To a solution of 84 (124.0 mg, 0.211 mmol) in anhydrous THF (2 mL) was added LiAlH₄ (1M, 0.158 mL, 0.158 mmol) at −78° C. After the reaction solution was stirred at −78° C. for 1 hour, it was quenched with ethyl acetate. Saturated aqueous potassium sodium tartrate (10 mL) was added. The organic layer was separated and the water layer was extracted with ethyl acetate (10 mL) three times. The combined organic layer was washed with brine and dried over Na₂SO₄. The solvent was removed and the product was isolated by silica gel column chromatography to afford 85 (120.5 mg. 97%) as white solid. ¹H NMR (360 MHz, C₆D₆): δ 5.35 (d. J=5.0 Hz, 1H), 4.17 (m, 2H), 3.84 (s, 1H), 3.59 (m, 1H), 3.40 (m, 1H), 3.31˜3.21 (m, 3H), 2.81 (q, J=7.3 Hz, 1H), 2.49 (m, 2H), 2.37 (dd, J=13.2, 3.0 Hz, 1H), 2.18 (td, J=12.7, 4.8 Hz, 1H), 2.05˜1.93 (m, 2H), 1.86 (d, J=12.7 Hz, 1H), 1.32 (d, J=7.2 Hz, 3H), 1.17 (s, 3H), 1.02 (s, 9H), 0.97 (s, 3H), 0.78 (d, J=6.5 Hz, 6H), 0.12 (s, 3H), 0.11 (s, 3H); ¹³C NMR (90 MHz, C₆D₆): δ 141.3, 121.7, 116.6, 86.9, 81.9, 72.9, 63.9, 62.8, 50.2, 48.4, 48.3, 43.5, 37.5, 36.8, 36.7, 34.8, 33.7, 33.2, 33.0, 32.7, 32.5, 32.4, 28.5, 26.1, 22.7, 22.3, 21.2, 19.5, 18.3, 13.0, 12.4, −4.3; HRMS (FAB) m/z 613.4252 (M+Na)⁺, calculated for C₃₅H₆₂O₅SiNa: 613.4264; [α]_{D}²⁴−35.7 (c 0.2, CHCl₃).

Z. Compound 86

A solution of 85 (18.0 mg, 0.03 mmol), 10 (58 mg, 0.061 mmol) and dry 4 Å MS powder (30 mg) in dry CH₂Cl₂ (0.7 mL) was stirred at 25° C. for 15 min, then was cooled to −20° C. TMSOTf (0.02M in CH₂Cl₂, 0.16 mL, 0.0032 mmol) was added. The reaction was stirred at −20° C. for 5 hours and was quenched with 0.1 mL Et₃N. The solid was filtered and the solvent was removed. The product was isolated with Et₃N deactivated silica gel column chromatography to afford 86 (30.1 mg, 71%, 89% of conversion yield) as colorless solid and recover 85 (3.7 mg, 21%). ¹H NMR (360 MHz, CDCl₃): δ 8.26 (d, J=8.8 Hz, 2H), 7.50 (d, J=8.4 Hz, 2H), 7.21 (d, J=8.6 Hz, 2H), 6.89 (d, J=8.4 Hz, 2H), 6.74 (d, J=8.6 Hz, 2H), 6.63 (d, J=8.8 Hz, 2H), 5.72 (s, 1H), 5.58 (broad s, 1H), 5.36 (broad s, 1H), 5.01 (s, 1H), 4.93 (d, J=11.3 Hz, 1H), 4.54 (d, J=11.3 Hz, 1H), 4.44 (d, J=11.0 Hz, 1H), 4.35 (d, J=11.0 Hz, 1H), 4.11 (dd, J=7.9, 4.2 Hz, 1H), 3.88 (m, 2H), 3.60 (m, 2H), 3.49 (t, J=7.1 Hz, 1H), 3.40 (s, 3H), 3.30 (s, 3H), 3.18 (s, 3H), 1.69 (s, 3H), 1.58 (d, J=7.0 Hz, 3H), 1.04 (s, 9H), 1.03 (s, 3H), 0.98 (t, J=7.8 Hz, 9H), 0.95 (s, 3H), 0.93 (d, J=6.5 Hz, 3H), 0.83 (d, J=6.3 Hz, 3H), 0.63 (q, J=7.8 Hz, 6H), 0.14 (s, 3H), 0.13 (s, 3H); ¹³C NMR (90 MHz, CDCl₃, taken at −30° C.): δ 169.0, 164.9, 163.5, 163.2, 158.7, 158.0, 140.1, 132.0, 130.1, 129.6, 128.0, 127.7, 127.4 (two peaks), 121.6, 121.5, 116.3, 113.4, 113.3, 112.9, 100.4, 94.7, 91.1, 86.7, 77.2, 72.5, 71.9, 71.0, 70.5, 70.2, 68.0, 66.2, 64.5, 63.7, 61.7, 59.4, 57.4, 55.8, 55.4, 55.2, 55.1, 54.9, 49.2, 47.7, 47.5, 42.5, 36.9, 36.0, 34.7, 34.3, 31.9, 31.7, 31.3, 30.8, 28.0, 25.9, 22.5, 21.2, 20.9, 20.4, 19.3, 18.5, 12.1, 6.8, 4.3, −4.9; LR MALDI m/z 1408.1 (M+Na)⁺, calculated for C₇₇H₁₁₆O₁₈Si₂Na: 1407.8; [α]_{D}²³+33.9 (c 1.3, CHCl₃).

AA. OSW-1 (1)

To a solution of 86 (16.5 mg, 0.012 mmol) in CH₂Cl₂—H₂O (1 mL, 10:1) was added DDQ (8.1 mg, 0.036 mmol). The reaction was stirred at 25° C. for 12 hours, then CH₂Cl₂ was removed and acetone (1 mL) and Pd(CN)₂Cl₂ (0.5 mg) was added. After the reaction was stirred at 25° C. for 2 hours, the solvent was removed and the product was isolated by preparative TLC to afford 1 (8.5 mg, 81%) as colorless solid. ¹H NMR (400 MHz, C₅D₅N): δ 8.33 (d, J=8.8 Hz, 2H), 7.09 (d, J=8.8 Hz, 2H), 5.69 (dd, J=9.1, 7.8 Hz, 1H), 5.57 (d, J=7.9, 6.3 Hz, 1H), 5.39 (d, J=3.9 Hz, 1H), 5.13 (d, J=7.6 Hz, 1H), 4.79 (s, 1H), 4.59 (d, J=6.1 Hz, 1H), 3.75 (s, 3H), 3.20 (q, J=7.5 Hz, 1H), 1.98 (s, 3H), 1.30 (d, J=7.4 Hz, 3H), 1.08 (s, 3H), 1.00 (s, 3H), 0.89 (d, J=6.2 Hz, 3H), 0.87 (d, J=6.2 Hz, 3H). ¹³C NMR (90 MHz, C₅D₅N): δ 218.9, 169.2, 165.4, 163.9, 149.3, 141.9, 132.4, 122.9, 121.1, 114.1, 103.7, 100.8, 88.3, 85.7, 81.0, 76.4, 75.1, 72.0, 71.3, 70.7, 67.9, 67.0, 65.6, 55.5, 50.1, 48.5, 46.5, 46.3, 43.5, 39.2, 37.7, 36.8, 32.7, 32.2, 32.0, 27.7, 22.8, 22.5, 20.9, 19.6, 13.6, 11.7; HRMS (FAB) m/z 895.4455 (M+Na)⁺, calculated for C₄₇H₆₈O₁₅Na: 895.4456, [α]_{D}²⁴−43 (c 0.3, CH₃OH).

Retrosynthetic Analysis. The C-20 carbon of OSW-1 has the “normal” 20S configuration. Molecular mechanics calculations (MM2) have shown that compound 1 is about 3.1 Kcal/mol more stable than its 20R epimer 6, whereas 7 is about 2.4 Kcal/mol more stable than 8 (Scheme 1). Therefore, the inventors thought that it was not necessary to control the stereochemistry at C-20 during the synthesis, and anticipated that compound 6 would eventually epimerize to the thermodynamically more stable 1 at the end of the synthesis.

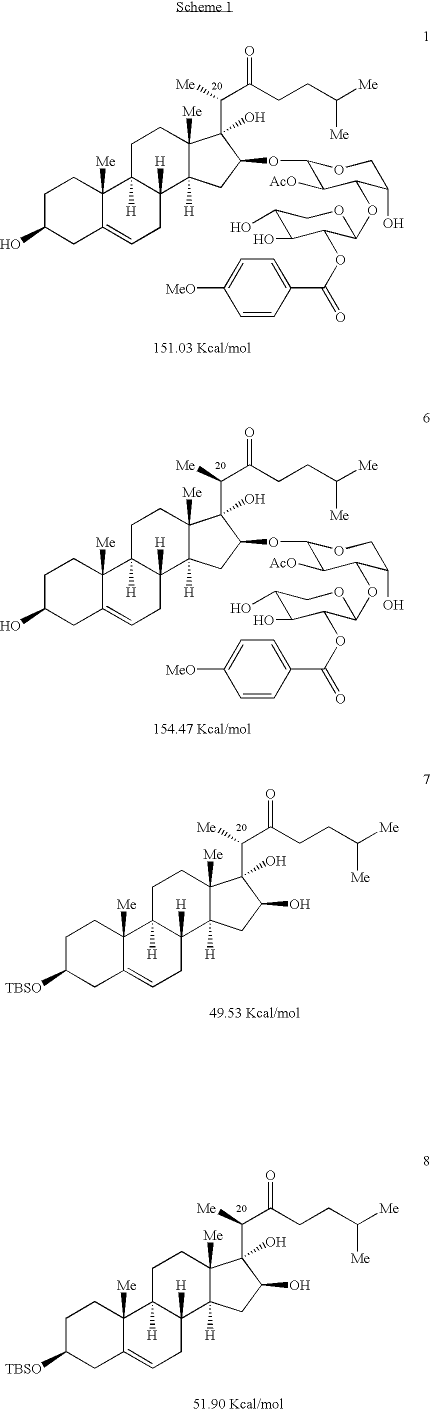

Scheme 2 outlines the retrosynthetic analysis of OSW-1 (1). Disconnection at the glycoside bond reveals the protected aglycone 9 and the disaccharide 10 as the potential key fragments for the construction of the target molecule. Compound 9 was envisioned to be formed via a triply convergent strategy which would involve 1,4-addition of acyl anion equivalent 11 to enone 12 followed by in situ stereoselective oxidation of the resulting enolate. Enone 12 was envisaged to be prepared from the commercially available steroid 14. Further disconnection at the glycoside bond of the disaccharide fragment 10 shows two monosaccharide units 15 and 16 which could be derived from L-arabinose and D-xylose, respectively.

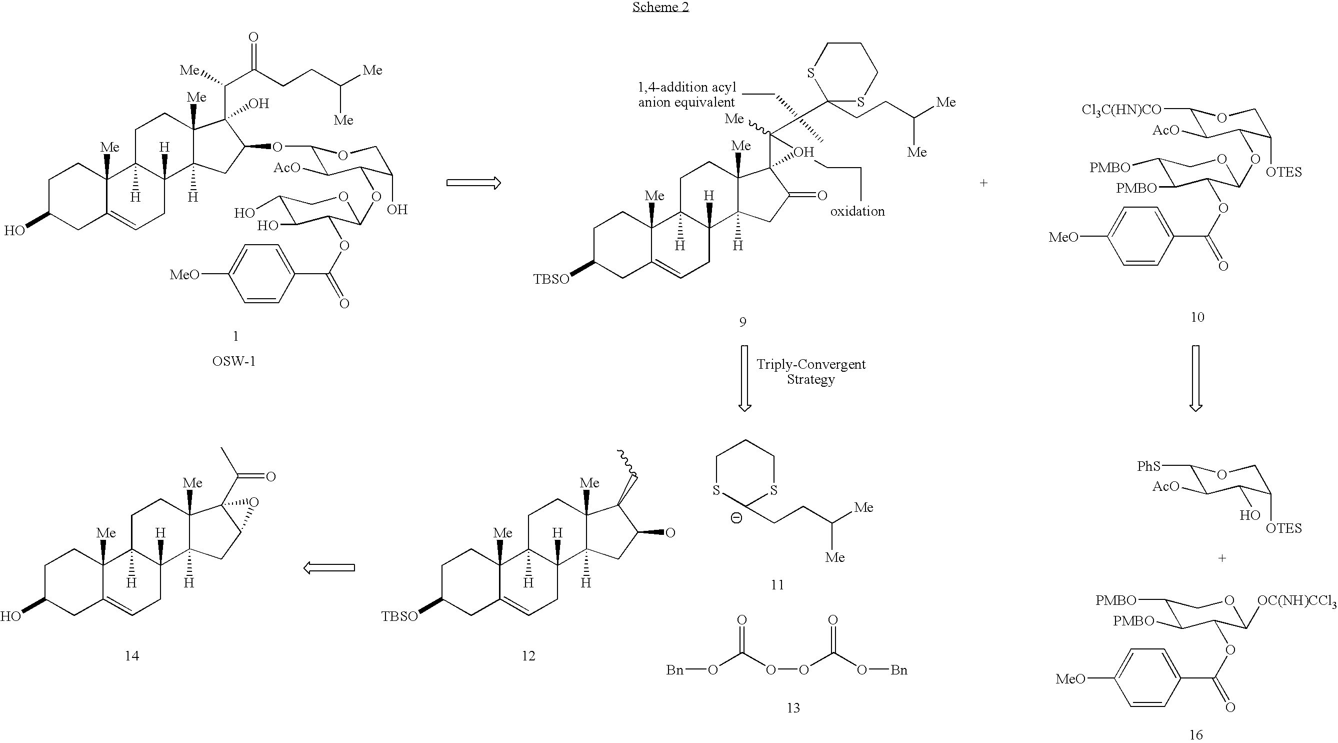

Synthesis of the Disaccharide 10. The first monosaccharide 15 was prepared from tetraacetyl-L-arabinose 17 as illustrated in Scheme 3. Thioglycoside 18 was prepared according to the standard methods (Nicolaou et al., 1997) followed by deacetylation to give compound 19 in excellent yield. Regioselective protection of the cis diol of 19 followed by protection of the C-2 hydroxyl group gave 20 in 90% yield. Deprotection of the acetonide afforded diol 21. It is well known that the equatorial C-3 hydroxyl group in many sugars is more reactive than C-4 axial hydroxyl group. High selectivity at C-4 hydroxyl group was observed when 21 was treated with TESOTf and lutidine at low temperature affording the desired product 15 in 90% yield.

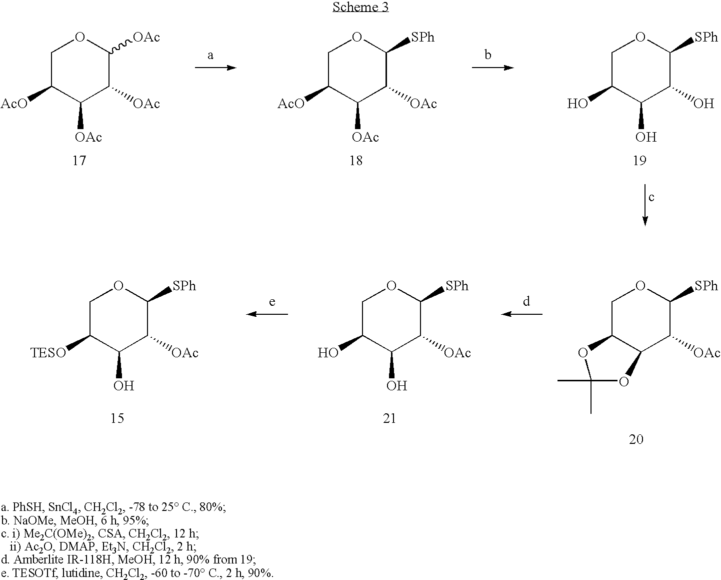

The second monosaccharide 16 was prepared from tetraacetyl-D-xylose 22. The thio orthoester 24 was prepared via the glycoside bromide 23 according to the literature procedures (Scheme 4). Protecting group manipulations followed by zinc chloride promoted intramolecular ring opening of the thio orthoester 26 gave thioglycoside 27 in excellent yield. After deacetylation, the p-methoxy benzoyl group was introduced at the C-2 position to afford 29, which was subsequently converted to 16 in 95% yield (Nicolaou et al., 1998). 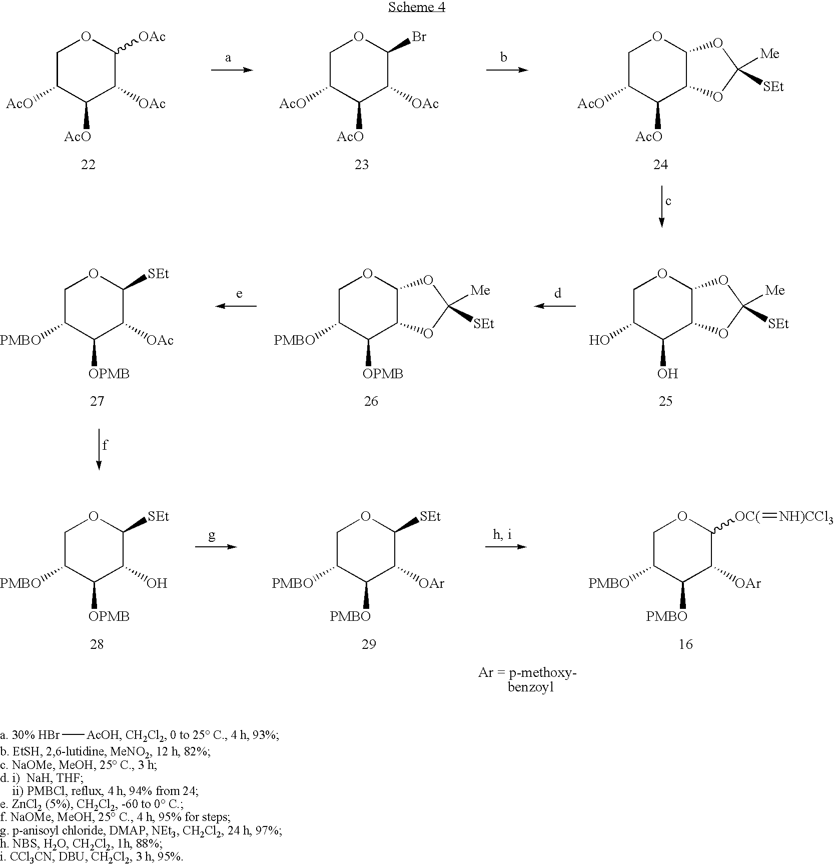
Glycosylation of 15 with 16 in the presence of BF₃.Et₂O afforded the β-disaccharide 30 in 93% yield (Scheme 5). Disaccharide 30 was then converted to the trichloroacetimidate 10, which was then ready to couple with the protected steroid aglycone.

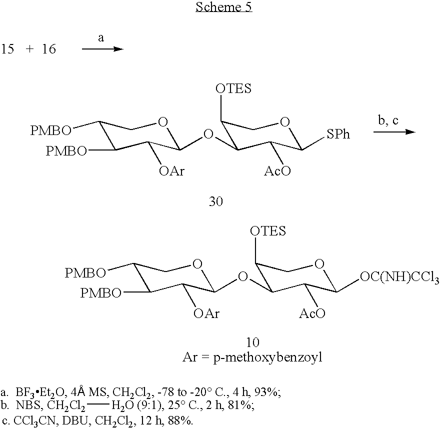

Attempted Synthesis of the Protected Steroid Aglycone. The commercially available 5-pregnen-16,17-epoxy-3β-ol-20-one 14 was protected by a TBS group to give compound 31 (Scheme 6). Reduction of the α,β-epoxy ketone 31 by hydrazine hydrate gave the allyl alcohol 32 in 73% yield (Kessar and Rampal, 1968; Kessar et al., 1968). Dess-Martin oxidation (Dess and Martin, 1991) of the allyl alcohol afforded 96% yield of enone 12 as a mixture of Z and E stereoisomers with a ratio of 2:1. The stereochemistry of the double bond was determined by NOSEY.

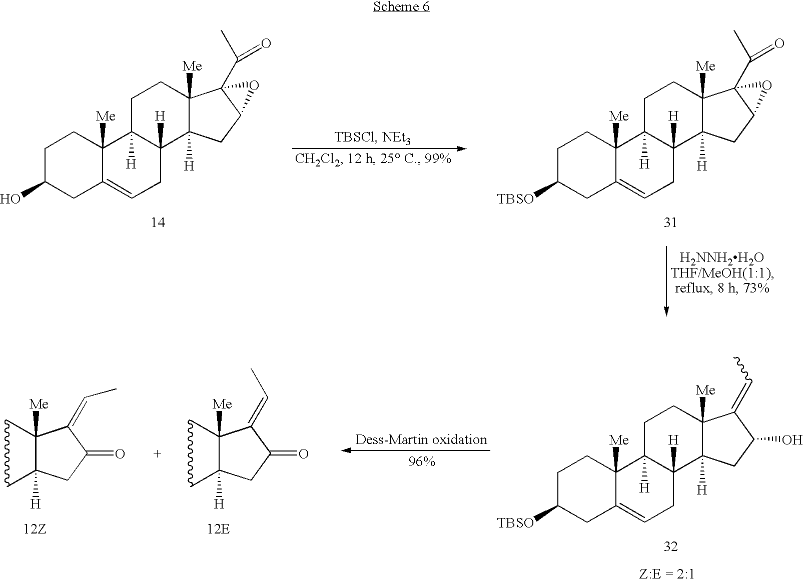

1,4-Addition of an acyl anion synthon to enone 12 was the key reaction to install the side chain of the aglycone in this strategy. Studies in the present invention on the addition of various α-thioacetal anions to enone 12 is summarized in Scheme 7.

The reaction between 1,3-dithiane anion 33 and enone 12 gave exclusively 1,2-addition product 35 even in the presence of HMPA and at room temperature (Scheme 7) (Reich and Sikorski, 1999; Brown et al., 1979). The softer anion 36 reacted with enone 12 in 1,2-fashion at −78° C. and then, in the presence of HMPA, rearranged to the 1,4-addition product 38 upon warming up the reaction mixture. However, the yield was quite low and 20% of the starting material enone was recovered due to the equilibrium. Anion 39 appeared to be the best choice, as it gave 65% yield of compound 41. Unfortunately, due to the steric hindrance of the tertiary anion 42, the reaction was too slow and the yield was quite low. Therefore, side chains were introduced in two steps via the addition of anion 39.

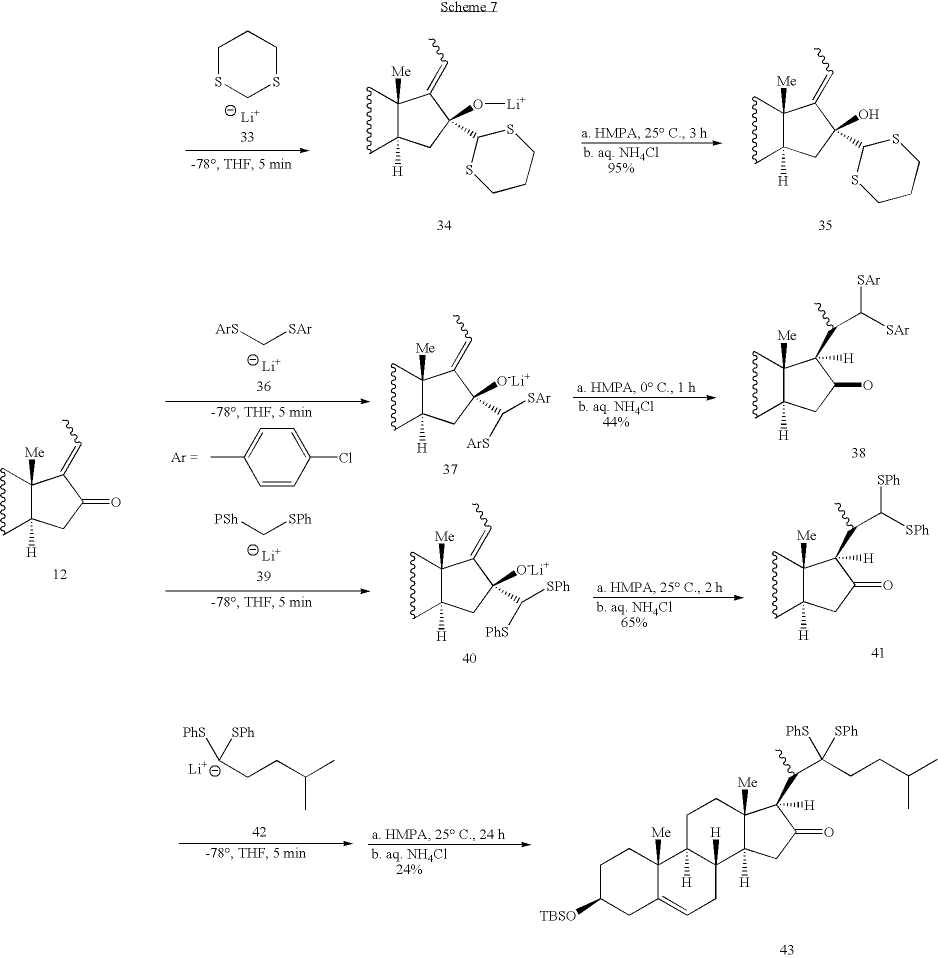

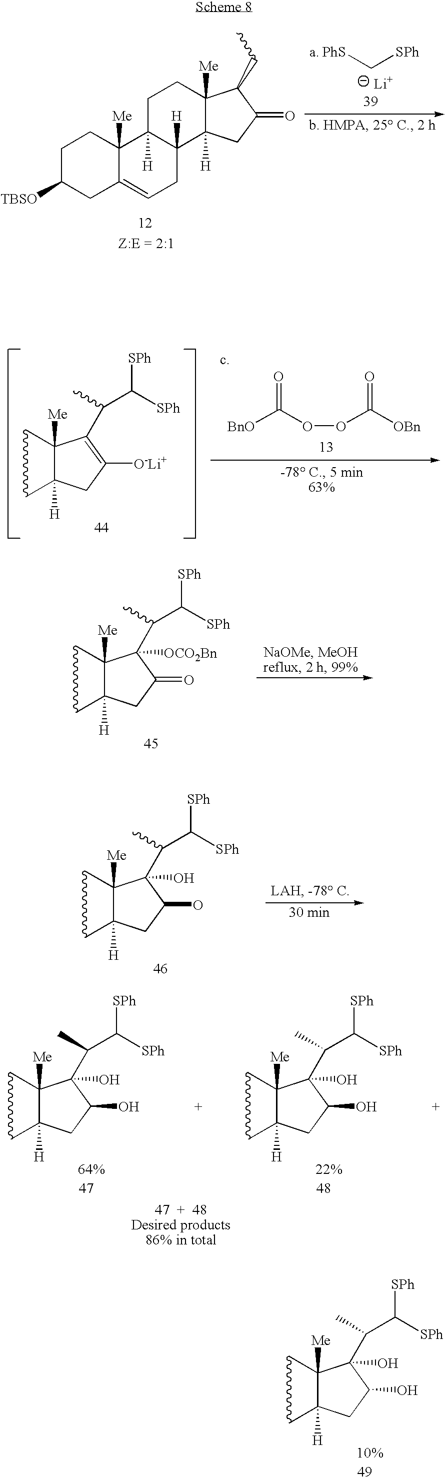

1,4 Addition of anion 39 to enone 12 afforded enolate intermediate 44, which was easily oxidized by dibenzyl peroxydicarbonate 13 (Gore and Vederas, 1986) at −78° C. to give compound 45 in 63% yield (Scheme 8). It appeared that the oxidation of the thioacetal by dibenzyl peroxydicarbonate 13 was much slower than the oxidation of the enolate at −78° C., and no sulfoxide product was isolated. Hydrolysis followed by stereoselective reduction by LiAlH₄ afforded three diastereoisomers 47, 48, and 49. The stereochemistry of the C-21 methyl group and the C-16 hydroxyl group were determined by analysis of the corresponding ROESY spectra. The desired products 47 and 48 which have β C-16 hydroxyl groups were obtained in a combined yield of 86%.

The metalation of both 47 and 48 proved to be quite difficult. After screening a few strong bases with or without additives such as HMPA or TMEDA, α-thioacetal anions 50 and 51 were successfully generated from 47 and 48 by treatment with super base (n-BuLi/t-BuOK) (Scheme 9) (Schlosser and Strunk, 1984). The formation of these anions was confirmed by deuterium incorporation after the reaction was quenched with D₂O at −78° C. Unfortanately, the attempt to quench them with electrophiles such as methyl iodide or allyl bromide resulted in quick decomposition of the anions 50 and 51. It is contemplated that the addition of electrophiles might accelerate the α-elimination of the highly bulky tertiary anions 50 and 51. This is supported by the fact that the reaction mixture smelled like thiophenol in the metalation step, and the odor of the thiophenol intensified immediately after the addition of an electrophile.

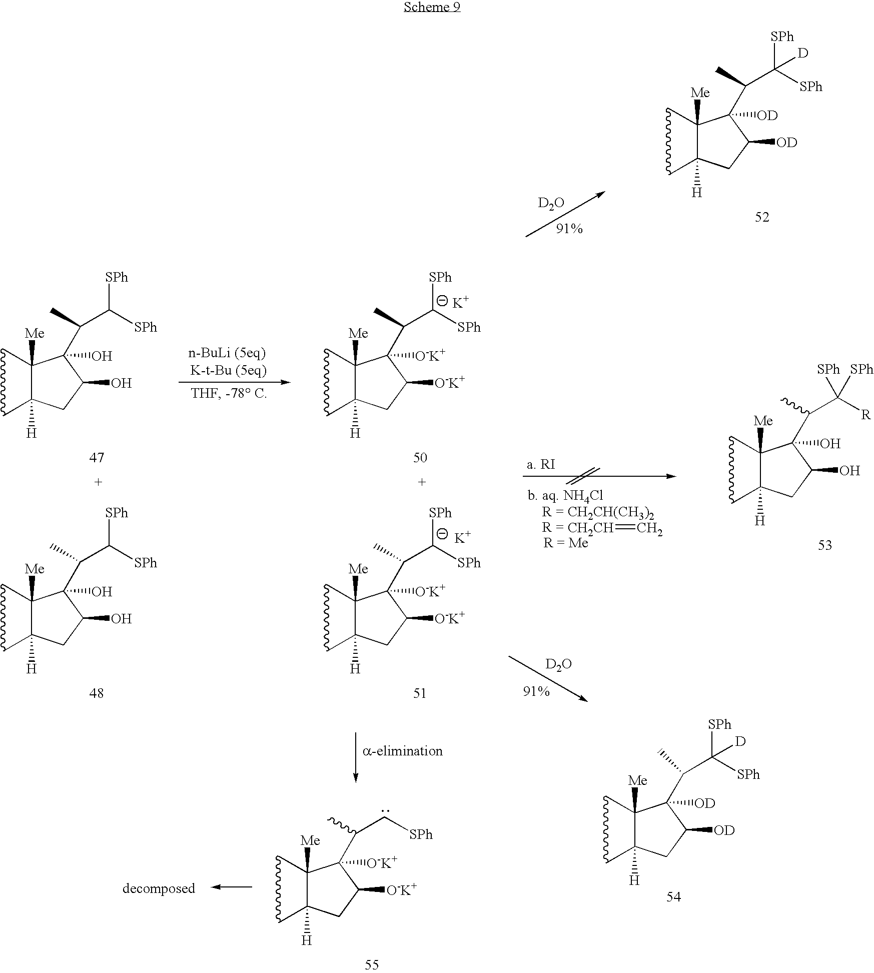

The unexpected difficulty in the alkylation of α-thioacetal anions 50 and 51, coupled with the difficulties in the 1,4-addition of the steric ally hindered tertiary α-thioacetal anions, led to modification of the original approach.

Attempted Synthesis of OSW-1. An α-alkoxy vinyl anion, such as anion 56, is another kind of acyl anion equivalent, which is more reactive and smaller compared to α-thioacetal anion 42 (Scheme 10). This suggests a new approach in which α-alkoxy vinyl anion can be employed as the acyl anion equivalent.

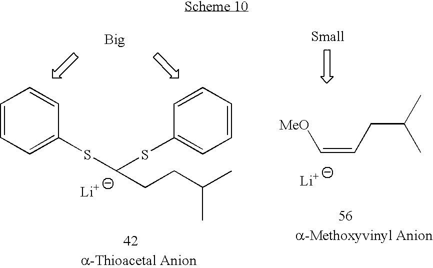

In the new approach, β-isobutyl substituted α-methoxy vinyl cuprate 58 was prepared (Scheme 11). However, there was no literature procedure for the quantitative generation of the requisite β-isobutyl substituted α-methoxy vinyl anion. To solve this problem, a new methodology for the regio- and stereoselective synthesis of α-halo vinyl ether that could serve as the precursor of the α-alkoxy vinyl anion was developed (Yu and Jin, 2000). The acetylenic ether 59 was prepared according to a literature procedure (Moyano et al., 1987). The α-bromovinyl ether 60 and the required α-methoxy vinyl cuprate 58 was prepared according to the newly developed methodology.

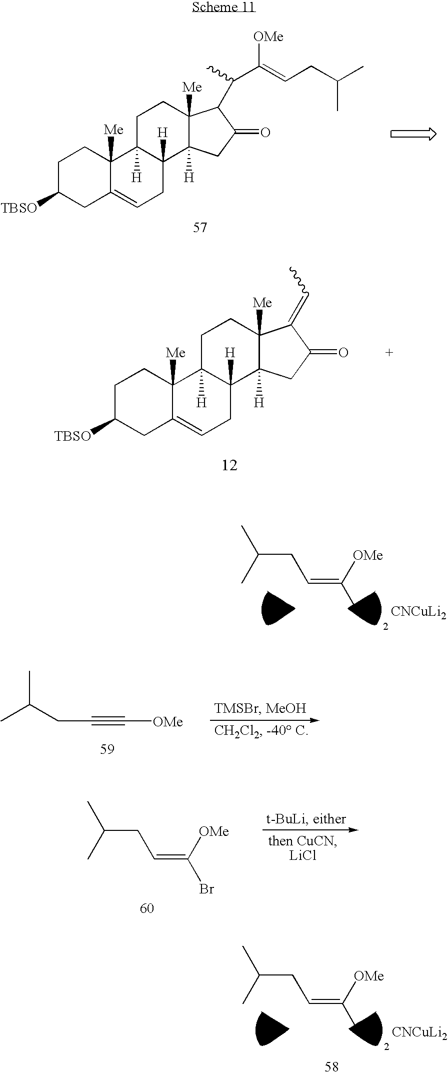

The inventors used compound 12Z (the major isomer of the enone mixture 12) to examine the proposed 1,4-addition reaction (Scheme 12). It was anticipated that the 12Z would be less reactive than the 12E isomer. Although there are several successful model studies on the 1,4-addition of cuprate 58 to various simple α,βp-unsaturated ketones, the reaction between cuprate 58 and enone 12Z did not lead to any desired product. Both low order and high order cuprates 61 and 58 were carefully examined (Lipshutz et al., 1984; Lipshutz, 1987), but no desired product was obtained even with TMSC1 activation (Corey and Boaz, 1985), Enone 12Z was recovered nearly quantitatively each time.

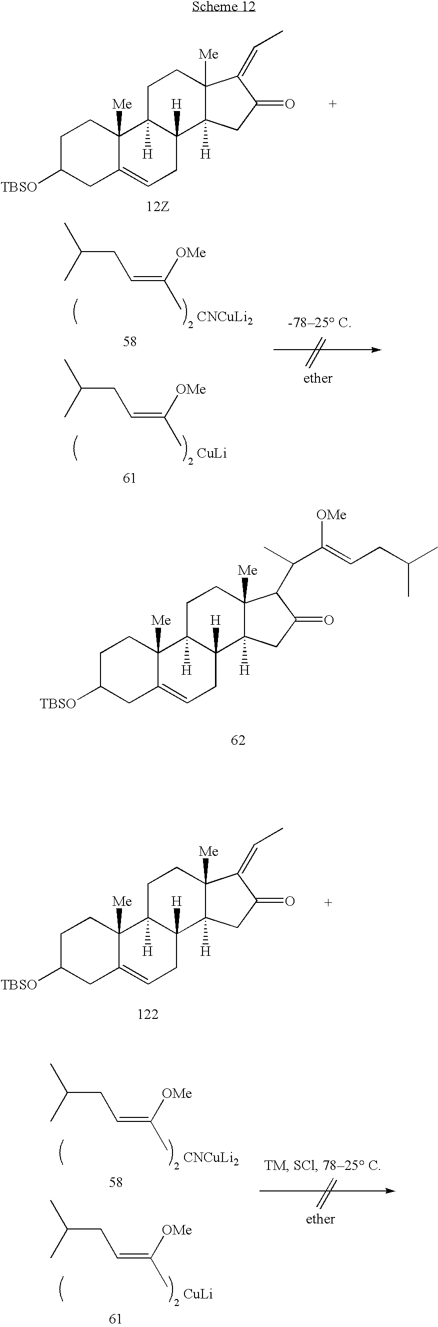

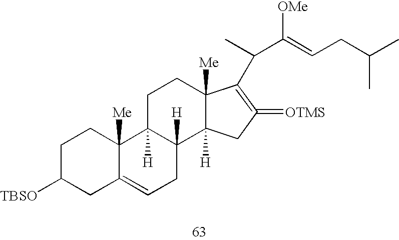

From the reaction mixture, a UV-active side product was isolated and it was found to be compound 64, which had obviously been formed via the Würtz coupling of the α-methoxy vinyl cuprate 58 (Scheme 13). The isolation of compound 64 suggested that the Würtz coupling was much faster than the desired 1,4 addition. Oxygen is normally considered to be the reason for the Würtz coupling side reaction of organocuprates (Blanchot-Courtois and Hanna, 1992). However, careful degassing of the reaction solvent and careful removal of possible trace of oxygen in argon by installing a Pyrogallol filter (Kim et al., 1999) still failed to stop the Würtz coupling.

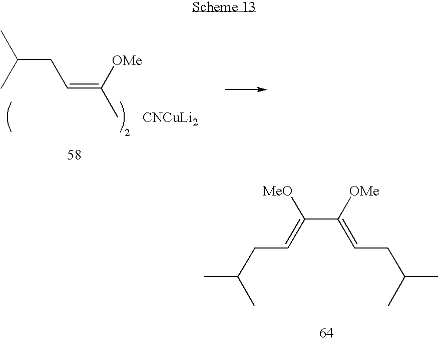

The two neighboring methoxy groups in the Würtz coupling product 64 are close to each other. Increasing the size of these two alkoxy groups was expected to suppress the formation of the Würtz coupling product. However, the alkoxy group should not be too bulky, otherwise the 1,4-addition would also be difficult. Based on the above analysis, α-cyclohexyloxy vinyl cuprate 68 was prepared. The size of the α-alkoxy group was increased from methoxy group to cyclohexyloxy group (Scheme 14).

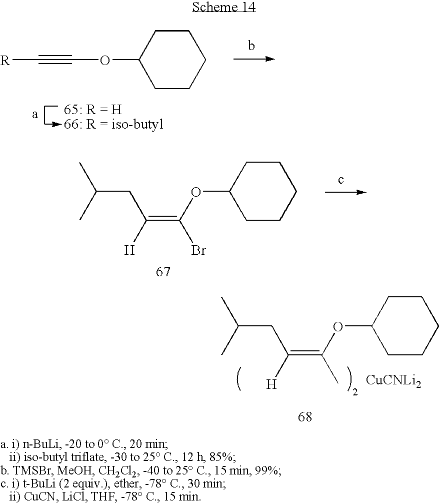

As expected, cuprate 68 underwent smooth 1,4-addition to enone 12Z in the presence of TMSC1 to afford the desired silyl enol ether 69 in 92% yield (Scheme 15). However, three equivalents of cuprate 68 were needed to drive the reaction to the completion.

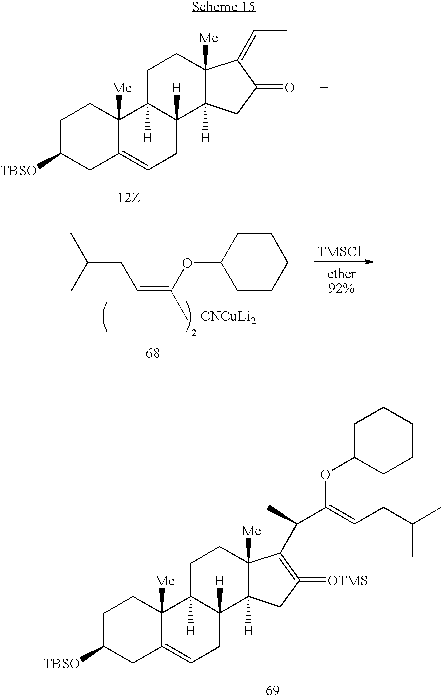

With the silyl enol ether 69 in hand, there was need to generate the enolate 70 and then oxidize the enolate 70 in situ to introduce the C-17 hydroxyl group (Scheme 16). The literature procedure using MeLi to cleave the silyl enol ether 69 was found to be extremely slow (Stork and Hudrlik, 1968). Some dry fluoride reagents were also employed, but none of them gave any satisfactory results. To solve this problem, a new methodology for the generation of enolates from silyl enol ethers by using potassium ethoxide was developed (Yu and Jin, 2001). Employing this new methodology, silyl enol ether 69 was cleaved in 5 minutes at 0° C. to give the potassium enolate 71 in quantitative yield.

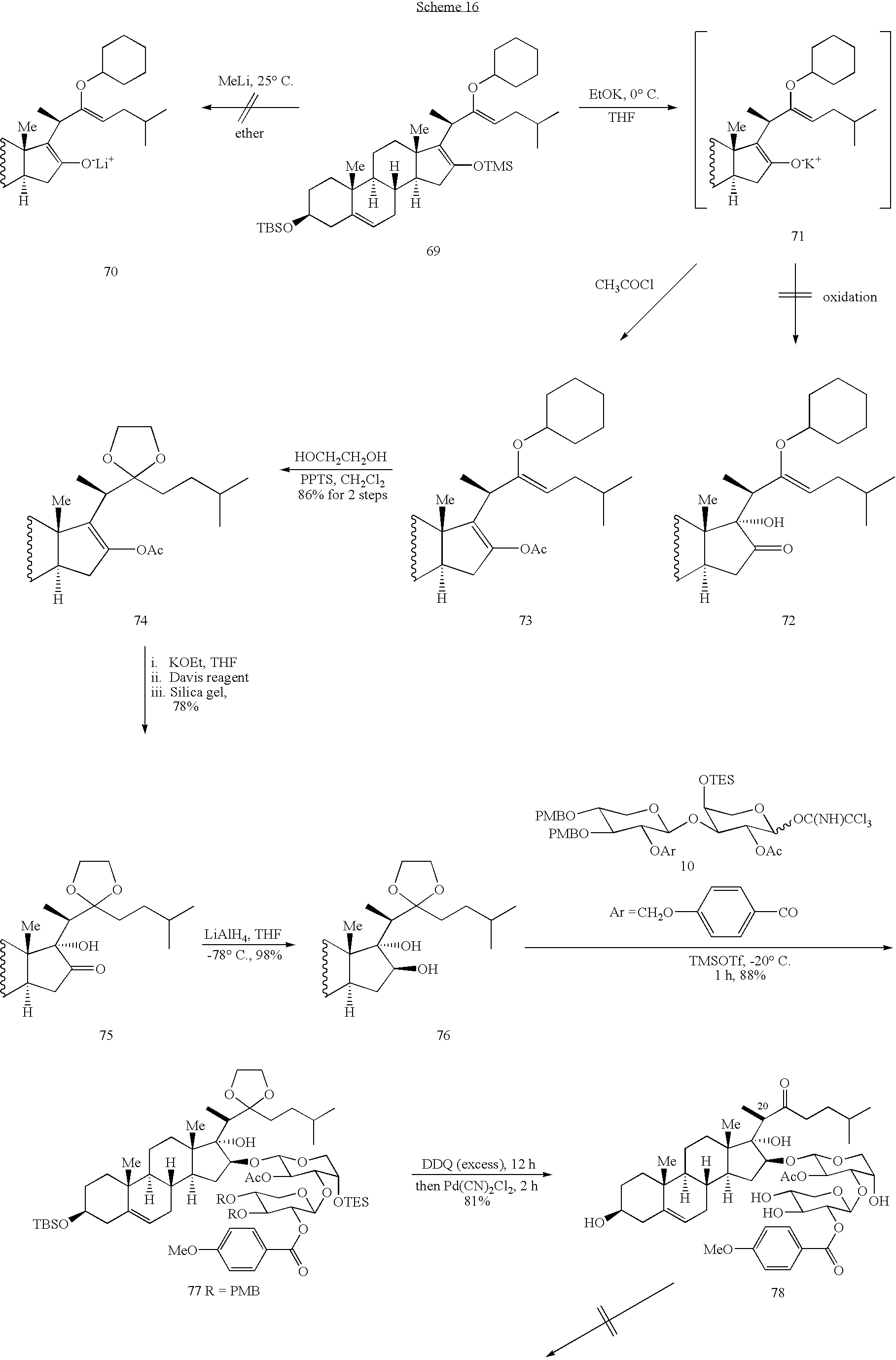

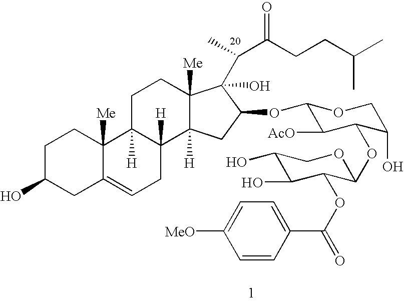

Efforts to oxidize enolate 71 with Davis reagent (Davis and Sheppard, 1989), molecular oxygen (Corey and Ensley, 1975), or dibenzyl peroxydicarbonate were unsuccessful. This problem is probably due to the presence of another labile enol ether moiety on the steroid side chain which is also prone to various oxidative reaction conditions. Thus, silyl enol ether 69 was converted to enol acetate 73, which enabled regiospecific convertion of the enol ether functionality at C-22 to cycloketal 74. Either EtOK or t-BuOK (Duhamel et al., 1993; Quesnel et al., 1998) was used to generate the enolate from enol acetate 74, and the enolate was then oxidized in situ by Davis reagent to give the α-hydroxyl ketone 75 in 78% yield. Stereospecific reduction of the C-16 ketone by LiAlH₄ at −78° C. afforded the trans diol 76 in 98% yield. The stereospecificity of the LiAlH₄ reduction was presumably due to the directing effort of C-17 hydroxy group.

Glycosylation of the diol 76 with the disaccharide 10 in the presence of TMSOTf provided β-glycoside 77 in 88% yield (Jiang and Schmidt, 1992). All the protecting groups, including two PMB, one TBS, one TES, and one cycloketal, were removed by sequential treatment with DDQ and Pd(CN)₂Cl₂ (Lipshutz et al., 1985) in a single operation to give 78 (C-20 epimer of OSW-1) in 81% yield.

To complete the total synthesis of OSW-1 (1), the stereochemistry of the C-20 methyl group needed to be epimerized to the requisite S-configuration. Although various basic conditions (pyridine, DBU, phosphazene base P₂-t-Bu (Schwesinger, 1987), etc.) and acidic conditions were investigated, the epimerization of C-20 methyl group to the S -configuration was not observed.

The reason for this stereochemistry problem at C-20 was directly related to the enone 12, a mixture of stereoisomers in favor of the undesired Z-isomer (Scheme 6). Therefore, a new approach was needed to synthesize enone 12E stereoselectively with the correct stereochemistry at C-20.

Total Synthesis of OSW-1. A new approach for the stereospecific synthesis of enone 12E was developed (Scheme 17). Compound 80 with the requisite Z-configuration was prepared according to a literature procedure from commercially available 5-androsten-3β-ol-17-one 79 (Schmuff and Trost, 1983). Selenium dioxide-mediated allylic oxidation provided 32E with complete chemo-, regio-, and stereoselectivity (Snider and Shi, 1999). Swern oxidation of 32E afforded enone 12E in nearly quantitative yield.

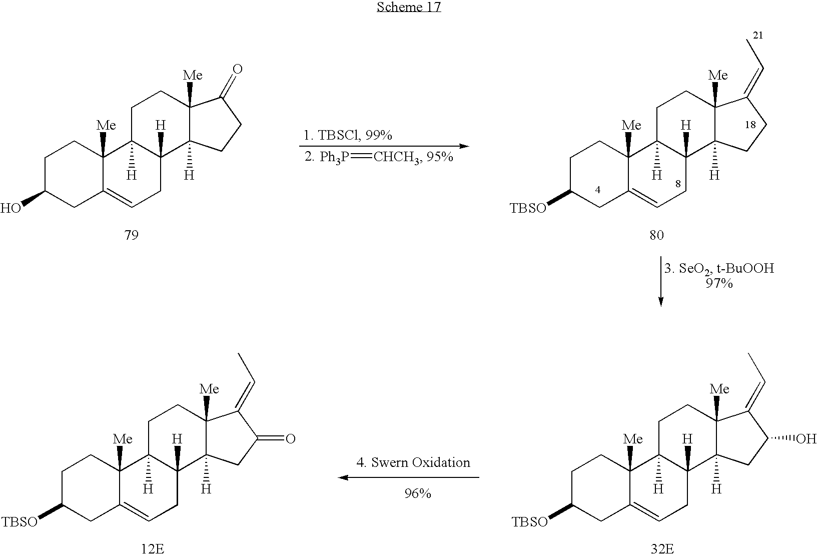

With enone 12E in hand, TMSC1 activated 1,4-addition of α-alkoxy vinyl cuprate 68 to enone 12E went smoothly to give silyl enol ether intermediate 81, which was further converted to enol acetate 82 without isolation of 81 (Scheme 18). Compound 82 was then converted to compound 83 in excellent yield. Generation of the enolate from 83 by potassium ethoxide or t-BuOK (Duhamel et al., 1993) followed by in situ stereoselective oxidation by Davis reagent (Davis and Sheppard, 1989) gave α-hydroxyl ketone 84 in 76% yield. Stereoselective reduction of 84 by LiAlH₄ at −78° C. provided the requisite trans 16β,17α-diol 85 in 97% yield. The stereochemistry at C16 and C17 of compound 85 was determined by NOESY spectra. Thus, the protected aglycone of OSW-1 (1) was synthesized with eight operations in 48% overall yield.

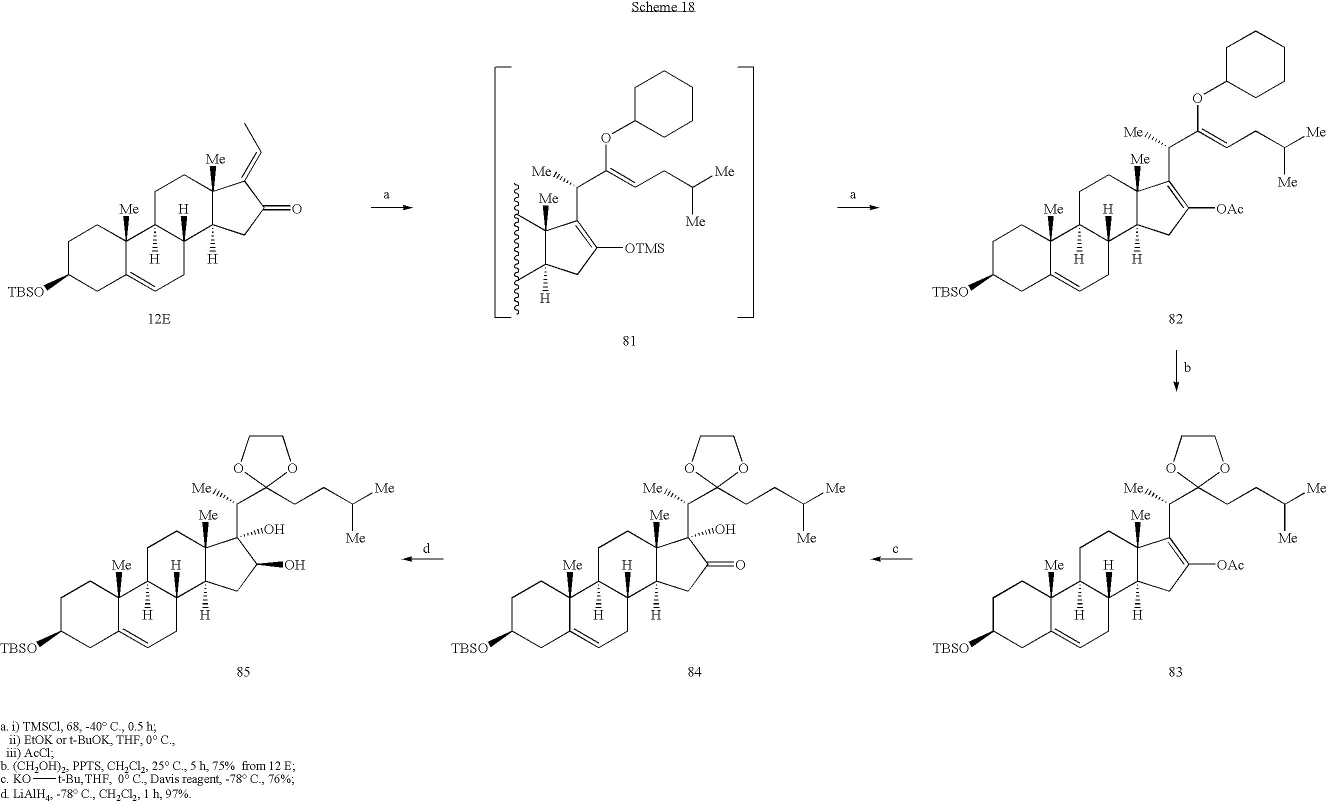

Coupling of disaccharide 10 with the steroid aglycone 85 under the standard conditions gave β-glycoside 86 in 71% yield. Removal of all the protecting groups by sequential treatment of compound 86 with DDQ and bis(acetonitrile)dichloro-palladium(II) in one operation afforded OSW-1 (1) in 81% yield. The physical data of synthetic OSW-1 (1) are identical to those reported by Sashida. 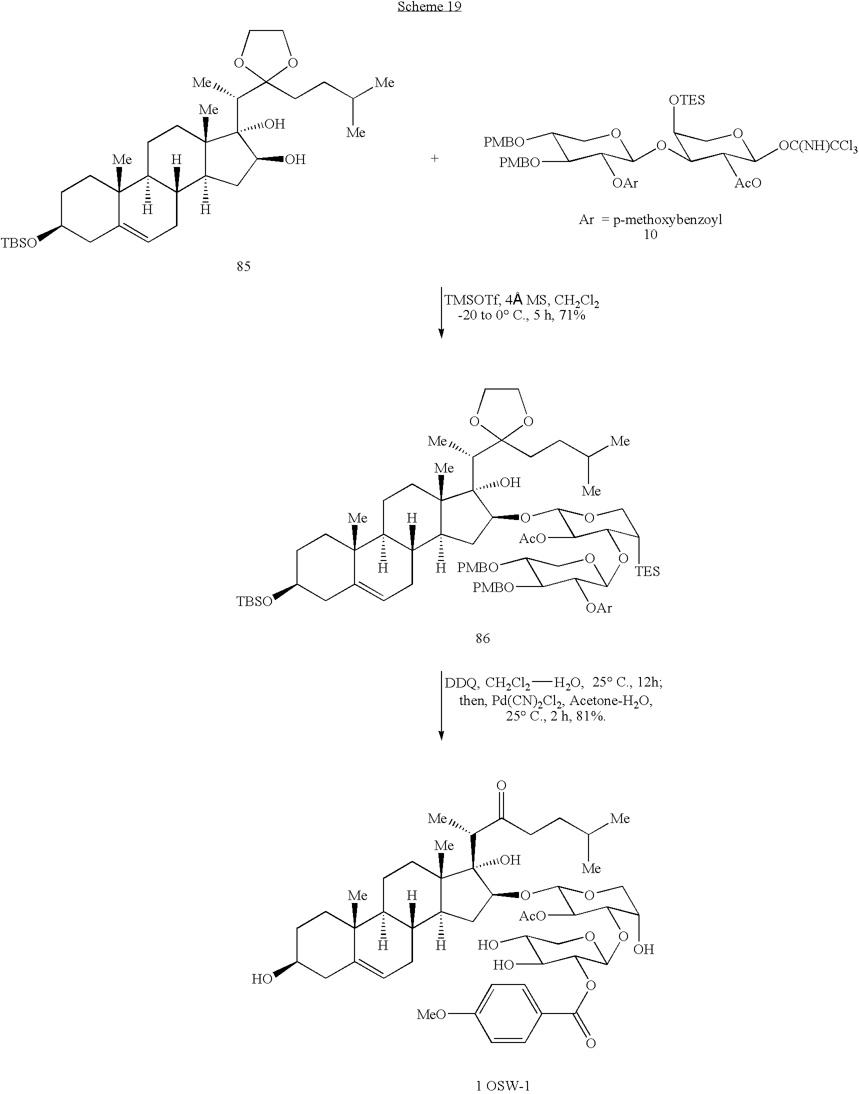
III. Therapies

The present invention provides methods for the treatment of various pancreatic cancers such as, but not limited to, ductal adenocarcinoma, mucinous cystadenocarcinoma, acinar carcinoma, unclassified large cell carcinoma, small cell carcinoma, pancreatoblastoma, intraductal papillary neoplasm, mucinous cystadnoma, and papillary cystic neoplasm and ovarian cancers such as, but not limited to, serous, mucinous, endometrioid, clear cell mesonephroid, Brenner, or mixed epithelial cancer as well as leukemias such as, but not limited to, CLL, AML and ALL and colon cancers. Other target cancers include cancers of the lung, brain, prostate, kidney, liver, ovary, breast, skin, stomach, esophagus, head and neck, testicles, cervix, lymphatic system and blood.

In some embodiments, the treatment methods will involve treating an afflicted individual with an effective amount of a composition comprising an orsaponin as described herein. An effective amount is described, generally, as that amount sufficient to detectably and repeatedly to induce apoptosis, induce cytotoxicity, inhibit cell division, inhibit metastatic potential, reduce tumor burden, increase sensitivity to chemotherapy or radiotherapy, kill a cancer cell, inhibit the growth of a cancer cell, or induce tumor regression.

To kill cells, induce apoptosis, inhibit cell growth, inhibit metastasis, decrease tumor size and otherwise reverse or reduce the malignant phenotype of tumor cells, using the methods of the present invention, one would generally contact a “target” cell or a “cancer” cell with the therapeutic composition comprising an orsaponin. This may be combined with compositions comprising other agents effective in the treatment of cancer. These compositions would be provided in a combined amount effective to kill or inhibit proliferation of the cancer cell.

A. Routes and Regimens

Contacting a target cell can be achieved by various routes of administration which include direct or local administration to a tumor, administration to the tumor vasculature, systemic administration, oral administration, topical administration, so that the orsaponin composition is ultimately delivered or contacted with a target cell. Thus, according to the present invention, one may treat the cancer by directly injection a tumor with an orsaponin composition. Alternatively, the tumor may be infused or perfused with the composition. Local or regional administration, with respect to the tumor, also is contemplated. Finally, systemic administration may be performed. Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Administration of the therapeutic orsaponin composition by the methods of the present invention to a patient will follow general protocols for the administration of chemotherapeutics, taking into account the toxicity, if any. It is expected that the treatment cycles would be repeated as necessary. It also is contemplated that various standard therapies, as well as surgical intervention, may be applied in combination with the treatments of the present invention.

Where clinical application of a composition is contemplated, it will be necessary to prepare an orsaponin composition as a pharmaceutical composition appropriate for the intended application. Generally this will entail preparing a pharmaceutical composition that is essentially free of pyrogens, as well as any other impurities that could be harmful to humans or animals. One also will generally desire to employ appropriate salts and buffers to render the complex stable and allow for complex uptake by target cells.

Depending on the particular cancer to be treated, administration of therapeutic compositions according to the present invention will be via any common route so long as the target tissue is available via that route. This includes oral, nasal, buccal, rectal, vaginal or topical. Topical administration would be particularly advantageous for treatment of skin cancers. Alternatively, administration will be by orthotopic, intraderrnal, subcutaneous, intramuscular, intraperitoneal or intravenous injection. Such compositions would normally be administered as pharmaceutically acceptable compositions that include physiologically acceptable carriers, buffers or other excipients.

The treatments may include various “unit doses.” Unit dose is defined as containing a predetermined-quantity of the therapeutic composition calculated to produce the desired responses in association with its administration, i.e., the appropriate route and treatment regimen. The quantity to be administered, and the particular route and formulation are within the skill of those in the clinical arts. Also of importance is the subject to be treated, in particular, the state of the subject and the protection desired. A unit dose need not be administered as a single injection but may comprise continuous infusion over a set period of time.

Preferably, patients will have adequate bone marrow function (defined as a peripheral absolute granulocyte count of >2,000/mm³ and a platelet count of 100,000/mm³), adequate liver function (bilirubin <1.5 mg/dl) and adequate renal function (creatinine <1.5 mg/dl).

In certain embodiments, the tumor being treated may not, at least initially, be resectable. Treatments with therapeutic orsaponin compositions may increase the resectability of the tumor due to shrinkage at the margins or by elimination of certain particularly invasive portions. Following treatments, resection may be possible. Additional treatments with the orsaponin compositions subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

B. Pharmaceutical Formulations

Aqueous compositions of the present invention comprise an effective amount of the orsaponin, such as OSW-1 or its derivatives, dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For human administration, preparations should meet sterility, pyrogenicity, general safety and purity standards as required by FDA Office of Biologics standards. The active compounds will generally be formulated for parenteral administration, e.g., formulated for injection via the intravenous, intramuscular, sub-cutaneous, intralesional, or even intraperitoneal routes. The preparation of an aqueous composition that comprises an orsaponin as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A composition of orsaponin can be formulated into a composition in a neutral or salt form. Pharmaceutically acceptable salts, include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of the pharmaceutical composition of the invention or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5.

In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations which are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.05% to 1%, or preferably 0.1%-0.2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.05% of active compound. The percentage of the compositions and preparations may, of course, be varied and may conveniently be between about 0.1% to about 1% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

C. Combination Cancer Therapies

In order to further enhance the efficacy of the chemotherapy provided by the invention, combination therapies are contemplated. Thus, a second anticancer therapeutic agent in addition to the orsaponin therapy of the invention may be used. The second therapeutic agent may be another chemotherapeutic agent, a therapeutic antibody, a radiotherapeutic agent, a gene therapeutic agent, a protein/peptide/polypeptide therapeutic agent, a hormonal agent, or an immunotherapeutic agent, etc. Such agents are well known in the art.

The administration of the second cancer therapeutic agent may precede or follow the orsaponin therapy by intervals ranging from minutes to days to weeks. In embodiments where the second therapeutic agent and a composition comprising an orsaponin are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the second therapeutic agent and/or the composition comprising an orsaponin will be required to achieve complete cancer cure. Various combinations may be employed, where the second therapeutic agent is “A” and the composition comprising an orsaponin is “B”, as exemplified below:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B
   Other combinations also are contemplated. The exact dosages and regimens of each agent can be suitable altered by those of ordinary skill in the art.

Provided below is a description of some other agents effective in the treatment of cancer.

(i) Radiotherapeutic Agents

Radiotherapeutic agents are known in the art to treat cancers. These agents include radiation and waves that induce DNA damage for example, γ-irradiation, X-rays, UV-irradiation, microwaves, electronic emissions, radioisotopes, and the like are contemplated. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

(ii) Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

(iii) Chemotherapeutic Agents

Agents that damage cancer cell DNA are chemotherapeutic agents. These can be, for example, agents that directly cross-link DNA, agents that intercalate into DNA, and agents that lead to chromosomal and mitotic aberrations by affecting nucleic acid synthesis. Agents that directly cross-link nucleic acids, specifically DNA, are envisaged and are exemplified by cisplatin, and other DNA alkylating agents. Agents that damage DNA also include compounds that interfere with DNA replication, mitosis, and chromosomal segregation.

Some examples of chemotherapeutic agents include antibiotic chemotherapeutics such as, Doxorubicin, Daunorubicin, Mitomycin (also known as mutamycin and/or mitomycin-C), Actinomycin D (Dactinomycin), Bleomycin, or Plicomycin. Plant alkaloids such as Taxol, Vincristine, Vinblastine. Miscellaneous agents such as Cisplatin, VP16, Tumor Necrosis Factor. Alkylating Agents such as, Carmustine, Melphalan (also known as alkeran, L-phenylalanine mustard, phenylalanine mustard, L-PAM, or L-sarcolysin, is a phenylalanine derivative of nitrogen mustard), Cyclophosphamide, Chlorambucil, Busulfan (also known as myleran), Lomustine. And other agents for example, Cisplatin (CDDP), Carboplatin, Procarbazine, Mechlorethamine, Camptothecin, Ifosfamide, Nitrosurea, Etoposide (VP16), Tamoxifen, Raloxifene, Estrogen Receptor Binding Agents, Gemcitabien, Navelbine, Farnesyl-protein transferase inhibitors, Transplatinum, 5-Fluorouracil, and Methotrexate, Temazolomide (an aqueous form of DTIC), or any analog or derivative variant of the foregoing.

(iv) Immunotherapy

Immunotherapeutics may be used in conjunction with the therapy using compositions comprising one or more orsaponins. Immunotherapeutics, generally, rely on the use of immune effector cells and molecules to target and destroy cancer cells. The immune effector may be, for example, another antibody specific for some other marker on the surface of a tumor cell. The antibody in itself may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells.

Many tumor markers exist and any of these may be suitable for targeting either the immune effector or even conjugating the orsaponin composition to a specific cancer type. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. Alternate immune stimulating molecules also exist including: cytokines such as IL-2, IL-4, IL-12, GM-CSF, gamma-IFN, chemokines such as MIP-1, MCP-1, IL-8 and growth factors such as FLT3 ligand.

(a) Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

(b) Active Immunotherapy

In active immunotherapy, an antigenic peptide, polypeptide or protein, or an autologous or allogenic tumor cell composition or “vaccine” is administered, generally with a distinct bacterial adjuvant (Ravindranath and Morton, 1991).

(c) Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and re-administered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro.

(v) Gene Therapy

In yet another embodiment, gene therapy is contemplated useful in conjunction with the anticancer methods of the invention that use compositions comprising an orsaponinin. A variety of nucleic acids and proteins encoded by nucleic acids are encompassed within the invention, some of which are described below. Table 1 lists various genes that may be targeted for gene therapy of some form in combination with the present invention.

TABLE 1

| | | | |
|---|---|---|---|
| Gene | Source | Human Disease | Function |

Growth Factors

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma | PDGF B |
| | virus | |

Receptor Tyrosine Kinases

| | | | |
|---|---|---|---|
| ERBB/HER | Avian | Amplified, deleted | EGF/TGF-α/ |
| | erythroblastosis | Squamous cell | Amphiregulin/ |
| | virus; ALV | Cancer; | Hetacellulin |
| | promoter | glioblastoma | receptor |
| | insertion; | | |
| | amplified | | |
| | human tumors | | |
| ERBB-2/NEU/HER-2 | Transfected from rat | Amplified breast, | Regulated by NDF/ |
| | Glioblastomas | Ovarian, gastric | Heregulin and |
| | | cancers | EGF- |
| | | | Related factors |
| FMS | SM feline sarcoma | | CSF-1 receptor |
| | virus | | |
| KIT | HZ feline sarcoma | | MGF/Steel receptor |
| | virus | | Hematopoieis |
| TRK | Transfection from | | NGF (nerve growth |
| | human colon | | Factor) receptor |
| | cancer | | |
| MET | Transfection from | | Scatter factor/HGF |
| | human | | Receptor |
| | osteosarcoma | | |
| RET | Translocations and | Sporadic thyroid | Orphan receptor Tyr |
| | point mutations | cancer; | Kinase |
| | | Familial medullary | |
| | | Thyroid cancer; | |
| | | Multiple endocrine | |
| | | Neoplasias 2A and | |
| | | 2B | |
| ROS | URII avian sarcoma | | Orphan receptor Tyr |
| | Virus | | Kinase |
| PDGF receptor | Translocation | Chronic | TEL(ETS-like |
| | | Myclomonocytic | transcription |
| | | Leukemia | factor)/ |
| | | | PDGF receptor |
| | | | gene |
| | | | Fusion |
| TGF-β receptor | | Colon carcinoma | |
| | | Mismatch mutation | |
| | | Target | |

NONRECEPTOR TYROSINE KINASES

| | | | |
|---|---|---|---|
| ABI. | Abelson Mul. V | Chronic | Interact with RB, |
| | | myelogenous | RNA |
| | | leukemia | polymerase, CRK, |
| | | translocation | CBL |
| | | with BCR | |
| FPS/FES | Avian Fujinami | | |
| | SV; GA | | |
| | FeSV | | |
| LCK | Mul. V (murine | | Src family; T cell |
| | leukemia | | signaling; interacts |
| | virus) promoter | | CD4/CD8 T cells |
| | insertion | | |
| SRC | Avian Rous | | Membrane- |
| | sarcoma | | associated Tyr |
| | Virus | | kinase with |
| | | | signaling function; |
| | | | activated by |
| | | | receptor kinases |
| YES | Avian Y73 virus | | Src family; |
| | | | signaling |

SER/THR PROTEIN KINASES

| | | |
|---|---|---|
| AKT | AKT8 murine | Regulated by |
| | retrovirus | PI(3)K; |
| | | regulate 70-kd S6 |
| | | k |
| MOS | Maloney murine SV | GVBD; cystostatic |
| | | factor; MAP |
| | | kinase |
| | | kinase |
| PIM-1 | Promoter insertion | |
| | Mouse | |
| RAF/MIL | 3611 murine SV; | Signaling in RAS |
| | MH2 | Pathway |
| | avian SV | |

MISCELLANEOUS CELL SURFACE¹

| | | | |
|---|---|---|---|
| APC | Tumor suppressor | Colon cancer | Interacts with |
| | | | catenins |
| DCC | Tumor suppressor | Colon cancer | CAM domains |
| E-cadherin | Candidate tumor | Breast cancer | Extracellular |
| | Suppressor | | homotypic |
| | | | binding; |
| | | | intracellular |
| | | | interacts with |
| | | | catenins |
| PTC/NBCCS | Tumor suppressor | Nevoid basal cell | 12 transmembrane |
| | and | cancer | domain; signals |
| | Drosophilia | Syndrome (Gorline | through Gli |
| | homology | syndrome) | homogue |
| | | | CI to antagonize |
| | | | hedgehog pathway |
| TAN-1 | Notch Translocation | T-ALI. | Signaling cell growth |
| homologue | | | and survival |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | Phosphorylated |
| | | | RING |
| | | | finger interact Abl |
| CRK | CT1010 ASV | | Adapted SH2/SH3 |
| | | | interact Abl |
| DPC4 | Tumor suppressor | Pancreatic cancer | TGF-β-related |
| | | | signaling |
| | | | Pathway |
| MAS | Transfection and | | Possible angiotensin |
| | Tumorigenicity | | Receptor |
| NCK | | | Adaptor SH2/SH3 |

GUANINE NUCLEOTIDE EXCHANGERS AND BINDING PROTEINS

| | | | |
|---|---|---|---|
| BCR | | Translocated with | Exchanger; protein |
| | | ABL in CML | Kinase |
| DBL | Transfection | | Exchanger |
| GSP | | | |
| NF-1 | Hereditary tumor | Tumor suppressor | RAS GAP |
| | Suppressor | Neurofibromatosis | |
| OST | Transfection | | Exchanger |
| Harvey-Kirsten, N- | HaRat SV; Ki | Point mutations in | Signal cascade |
| RAS | RaSV; | many | |
| | Balb-MoMuSV; | Human tumors | |
| | Transfection | | |
| VAV | Transfection | | S112/S113; |
| | | | exchanger |

NUCLEAR PROTEINS AND TRANSCRIPTION FACTORS

| | | | |
|---|---|---|---|
| BRCA1 | Heritable suppressor | Mammary | Localization |
| | | Cancer/ovarian | unsettled |
| | | cancer | |
| BRCA2 | Heritable suppressor | Mammary cancer | Function unknown |
| ERBA | Avian | | Thyroid hormone |
| | erythroblastosis | | receptor |
| | Virus | | (transcription) |
| ETS | Avian E26 virus | | DNA binding |
| EVII | MuLV promotor | AML | Transcription factor |
| | Insertion | | |
| FOS | FBI/FBR murine | | 1 transcription |
| | osteosarcoma | | factor |
| | viruses | | with c-JUN |
| GLI | Amplified glioma | Glioma | Zinc finger; cubitus |
| | | | interruptus |
| | | | homologue |
| | | | is in hedgehog |
| | | | signaling pathway; |
| | | | inhibitory link |
| | | | PTC |
| | | | and hedgehog |
| HMGI/LIM | Translocation | Lipoma | Gene fusions high |
| | t(3:12) | | mobility group |
| | t(12:15) | | HMGI-C (XT- |
| | | | hook) |
| | | | and transcription |
| | | | factor |
| | | | LIM or acidic |
| | | | domain |
| JUN | ASV-17 | | Transcription factor |
| | | | AP-1 with FOS |
| MLL/VHRX + | Translocation/fusion | Acute myeloid | Gene fusion of |
| ELI/MEN | ELL with MLL | leukemia | DNA- |
| | Trithorax-like gene | | binding and methyl |
| | | | transferase MLL |
| | | | with |
| | | | ELI RNA pol II |
| | | | elongation factor |
| MYB | Avian | | DNA binding |
| | myeloblastosis | | |
| | Virus | | |
| MYC | Avian MC29; | Burkitt's lymphoma | DNA binding with |
| | Translocation B- | | MAX partner; |
| | cell | | cyclin |
| | Lymphomas; | | regulation; interact |
| | promoter | | RB; regulate |
| | Insertion avian | | Apoptosis |
| | leukosis | | |
| | Virus | | |
| N-MYC | Amplified | Neuroblastoma | |
| L-MYC | | Lung cancer | |
| REL | Avian | | NF-κB family |
| | Retriculoendotheliosis | | Transcription factor |
| | Virus | | |
| SKI | Avian SKV770 | | Transcription factor |
| | Retrovirus | | |
| VHL | Heritable suppressor | Von Hippel-Landau | Negative regulator |
| | | Syndrome | or elongin; |
| | | | transcriptional |
| | | | elongation |
| | | | complex |
| WT-1 | | Wilm's tumor | Transcription factor |

CELL CYCLE/DNA DAMAGE RESPONSE¹⁰⁻²¹

| | | | |
|---|---|---|---|
| ATM | Hereditary disorder | Ataxia- | Protein/lipid kinase |
| | | telangiectasia | Homology; DNA |
| | | | damage response |
| | | | upstream in P53 |
| | | | pathway |
| BCL-2 | Translocation | Follicular | Apoptosis |
| | | lymphoma | |
| FACC | Point mutation | Fanconi's anemia | |
| | | group | |
| | | C (predisposition | |
| | | Leukemia | |
| MDA-7 | Fragile site 3p14.2 | Lung carcinoma | Histidine triad- |
| | | | related |
| | | | Diadenosine |
| | | | 5′,3″″- |
| | | | tetraphosphate |
| | | | Asymmetric |
| | | | hydrolase |
| hMLI/MutL | | HNPCC | Mismatch repair; |
| | | | MutL |
| | | | Homologue |
| hMSH2/MutS | | HNPCC | Mismatch repair; |
| | | | MutS |
| | | | Homologue |
| hPMS1 | | HNPCC | Mismatch repair; |
| | | | MutL |
| | | | Homologue |
| hPMS2 | | HNPCC | Mismatch repair; |
| | | | MutL |
| | | | Homologue |
| INK4/MTS1 | Adjacent INK-4B at | Candidate MTS1 | P16 CDK inhibitor |
| | 9p21; CDK | suppressor and | |
| | complexes | MLM | |
| | | Melanoma gene | |
| INK4B/MTS2 | | Candidate | p15 CDK inhibitor |
| | | Suppressor | |
| MDM-2 | Amplified | Sarcoma | Negative regulator |
| | | | p53 |
| p53 | Association with | Mutated >50% | Transcription factor; |
| | SV40 | human | Checkpoint control; |
| | T antigen | Tumors, including | DNA damage response; |
| | | Hereditary Li- | apoptosis |
| | | Fraumeni | |
| | | Syndrome | |
| PRAD1/BCL1 | Translocation with | Parathyroid | Cyclin D |
| | Parathyroid | adenoma; | |
| | hormone or IgG | B-CLL | |
| RB | Hereditary | Retinoblastoma; | Interact cyclin/cdk; |
| | Retinoblastoma; | Osteosarcoma; | regulate E2F |
| | Association with | breast | transcription factor |
| | many | Cancer; other | |
| | DNA virus tumor | sporadic | |
| | Antigens | Cancers | |
| XPA | | Xeroderma | Excision repair; |
| | | Pigmentosum; skin | photo- |
| | | Cancer | product |
| | | predisposition | recognition; |
| | | | zinc finger |

(vi) Other Agents

It is contemplated that other agents may be used in combination with the present invention to improve the therapeutic efficacy of treatment. One form of therapy for use in conjunction with chemotherapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention. The use of hormones may be employed in the treatment of certain cancers such as breast, prostate, ovarian, or cervical cancer to lower the level or block the effects of certain hormones such as testosterone or estrogen and this often reduces the risk of metastases.

The terms “contacted” and “exposed,” when applied to a cell, are used herein to describe the process by which a therapeutic construct or protein and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cells and Cell Lines. Various cancer cell lines were used including the human leukemia cell lines HL-60 and ML-1 cells; human lymphoma cell line Raji; human pancreatic cancer cells AsPC-1; and human ovarian cancer cells SKOV3; human colon cancer cells HCT116; as well as isolated CLL cells from patients; and lymphocytes from normal donors. Cells were grown and maintained as per protocols known in the art using appropriate cell culture media and supplements.

Assay for In Vitro Evaluation. A variety of in vitro assays were used to determine the response of cancer cells to the orsaponin compositions and formulations of the present invention. Some of the responses assayed included cell viability, apoptosis, and cell killing. Provided below is a brief description of these assays:

1. MTT Assays. For these assays either cancer cell lines were used. Alternatively, mononuclear cells from peripheral blood samples of CLL patients or normal donors were separated by Ficoll Hipaque fractionation and resuspended in DMEM complete medium. Malignant cells from various human cell lines (usually at 5×10⁴ cells/ml) or mononuclear cells from peripheral blood of leukemia patients and healthy donors (1×10⁶ cells/ml) were incubated in either αMEM or RPMI 1640 with or without various concentrations of OSW-1 in the range of 0-10 nM. Each experimental condition was done in triplicate. After the indicated number of days (usually 3 days or 72 hours) of exposure to OSW-1, cell survival was assessed by the addition of the MTT dye to the wells. The MTT dye changes its color depending on the presence of live cells in the well. Survival of cells under orsaponin treatment was evaluated as a percentage of control cell growth. Alternatively, one can also use the dye trypan-blue which penetrates dead cells thereby allowing one to count live cells under the microscope and estimating percentage survival.

2. Analysis of Apoptosis. Two methods were used to analyze apoptosis by assaying different events in the apoptotic pathways. Percentages of apoptotic cells induced by orsaponins of the invention were evaluated using flow cytometer. Different methods of staining cells for apoptosis were utilized to assess different aspects of apoptotic cascade.

(i) Annexin V Staining. Annexin V binds to cells that express phosphatidylserine on the outer layer of the cell membrane. This allows live cells (unstained cells) to be discriminated from apoptotic cells (stained with annexin V). Following treatment of cells in culture with different concentrations of the OSW-1 composition for the indicated time, cells were washed in phosphate-buffered saline (PBS) and resuspended in 100 μl of binding buffer containing annexin V-FITC (Travigene) and incubated for 15 minutes in the dark. Cells were analyzed on flow cytometer.

(ii) DNA Fragmentation Assay. Cancer cells are incubated with or without OSW-1. After incubation for different time periods, 0, 8, 16, 24 and 32 hours, the cells are harvested and washed with 1×PBS. The cell are then lysed in a solution containing 10 mM Tris-HCl, pH 7.5, 25 mM EDTA, 0.5% SDS, and 0.1 mg protease K. After digestion and removal of the cellular protein from the cellular DNA, the isolated DNA is analyzed on a 1.8% agarose gel, and visualized by ethidium bromide staining. DNA that was cleaved/degraded into fragments, as a result of apoptosis due to treatment with orsaponin, can be visualized on a gel as fragmented DNA bands of various molecular weights.

3. Analysis of the Mitochondrial Transmembrane Potential. To evaluate the role of mitochondrial respiration in the cytotoxicity of OSW-1, changes in the potential of mitochondrial membrane were analyzed. Following the treatment of cells with OSW-1 for different time periods, cells were incubated with rhodamine-123 which is a potential-sensitive dye. Cytometric analysis was used to record changes in mitochondrial potential. These assays were performed on the parental HL-60 lukemia cells and its mutant line C6F which has a mitochondrial respiration defect, as well as on parental ML-1 cells, which are cells derived from human myeloblastic leukemia, and its mutant cell line C, 19 which has a mitochondrial respiration defect.

### Example 2

### Induction of Cytotoxicity and Apoptosis

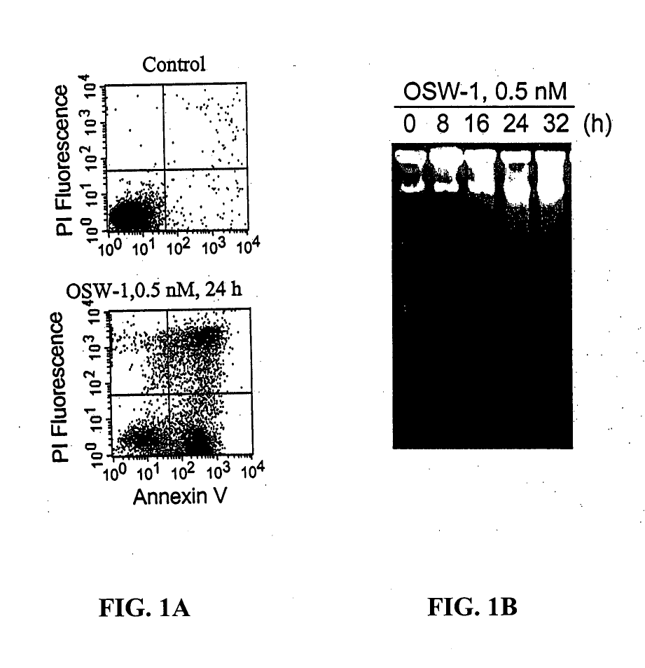

OSW-1 Induces Apoptosis. Orsaponin (OSW-1) induces apoptosis human leukemia cells. For example, HL-60 cells in exponentially growing phase were treated with 0.5 nM OSW-1 for 0, 8, 16, 24 and 32 hours respectively. Drug induced apoptosis was analyzed by both annexin V assay (see data in FIG. 1) as well as by the DNA fragmentation assay (FIG. 1B), which shows that apoptosis occurred 16 h after incubation with orsaponin. This lagging period likely reflects the time needed for orsaponin to interact with its cellular target molecule and trigger the down stream apoptotic cascade, and suggests that orsaponin does not directly lyze or damage cellular membranes.

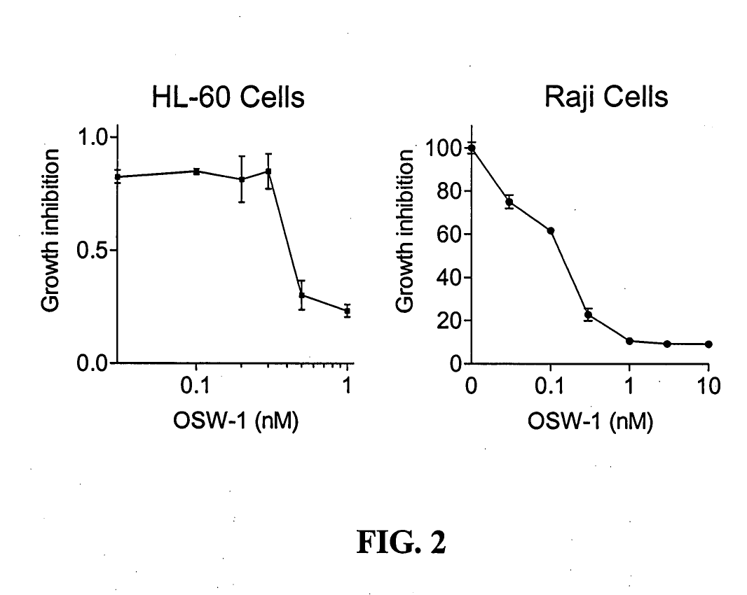

OSW-1 Inhibits Growth of Leukemic Cells. OSW-1 inhibits cell growth in several human leukemia and lymphoma cell lines including HL-60 and Raji as measured by the MTT assay. OSW-1 inhibited growth in these cells in a dose-dependent manner, over a range of 0-1.0 nM (see FIG. 2). OSW-1 is highly potent and shows cell growth inhibition with an IC₅₀ value below 0.5 nM.

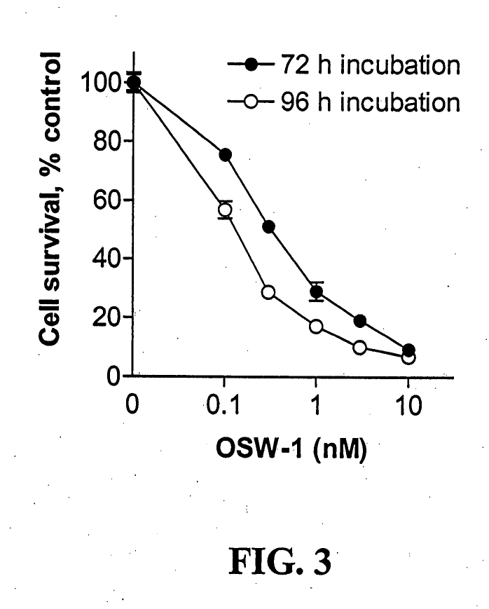

OSW-1 Inhibits Growth of Pancreatic Cancer Cells. Cell growth inhibition by OSW-1 was also seen in the human pancreatic cancer cells AsPC-1. These cells were treated with the 0-10 nM OSW-1 for 72 h or 96 h and cell growth inhibition was measured by the MTT assay. The results are depicted in FIG. 3 and show that OSW-1 is highly potent at concentrations of 0.1-5 nM.

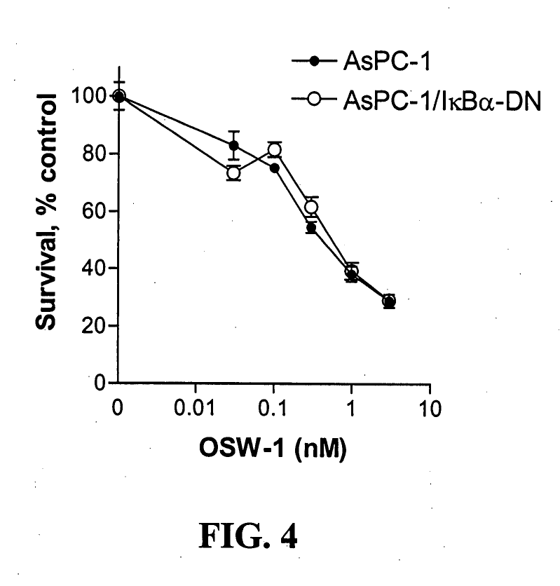

OSW-1 Inhibits Growth of Pancreatic Cancer Cells Irrespective of NFκB Expression. It is also demonstrated that the anticancer growth inhibitory activity of Orsaponin (OSW-1) is not affected by NFκB expression in pancreatic cancer cells (see FIG. 4). Human pancreatic cancer cells AsPC-1 with constitutive activation of NFκB or inactivation of NFκB by dominant negative IκBα (AsPC-1/IκBα-ND) were treated with the 0-10 nM OSW-1 for 72 h. Cell growth inhibition was measured by MTT assay. It is obvious that both cell lines exhibit similar sensitivity to OSW-1 with an IC₅₀ value of approximately 0.3 nM, indicating that the NFκB activation status does not affect the cytotoxic action of orsaponin. Thus, orsaponin may be used to effectively treat drug-resistant pancreatic cancers with constitutive activation of NFκB.

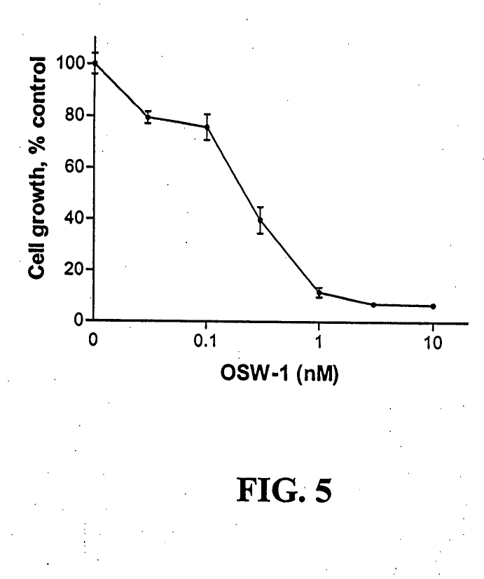

OSW-1 Inhibits Growth of Ovarian Cancer Cells. OSW-1 inhibited cell growth in human ovarian cancer cells SKOV3, see FIG. 5. Human ovarian cancer SKOV3 cells were treated with 0-10 nM OSW-1 for 72 h and cell growth inhibition was measured by the MTT assay.

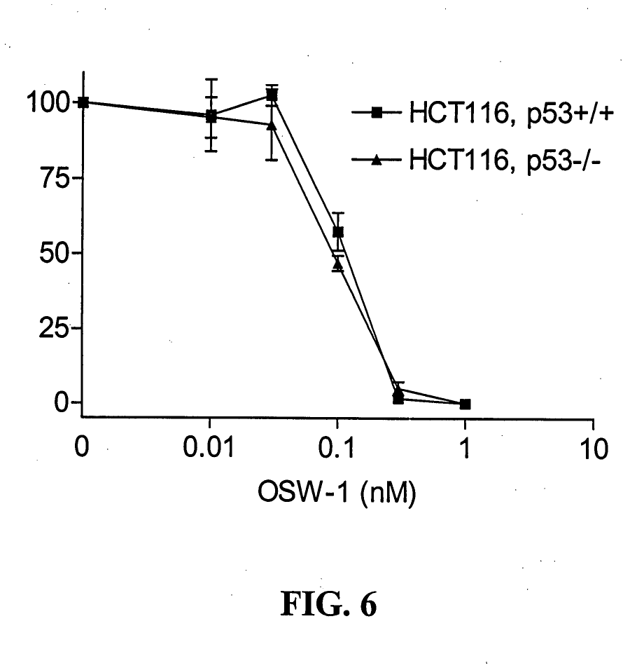

OSW-1 Inhibits Growth of Human Colon Cancer Cells. OSW-1 was shown to effectively inhibit the ability of human colon cancer cells to form colonies in culture, see FIG. 6. The HCT116 colon cancer cells with wild-type p53 (p53+/+) and p53-null (p53−/−) were treated with the various concentrations of orsaponin (OSW-1), and cell survival was measured by colony formation assay. The results indicate that orsaponin is effective in killing human colon cancer cells regardless of p53 expression status. Since mutations and defects in p53 function occurs in the majority of human cancers and is associated with drug resistance to many anticancer agents currently used in the clinic, the ability of orsaponin to effectively kill the p53−/− cells indicates the therapeutic utility of OSW-1 in treating various types of human cancers with p53 mutations.

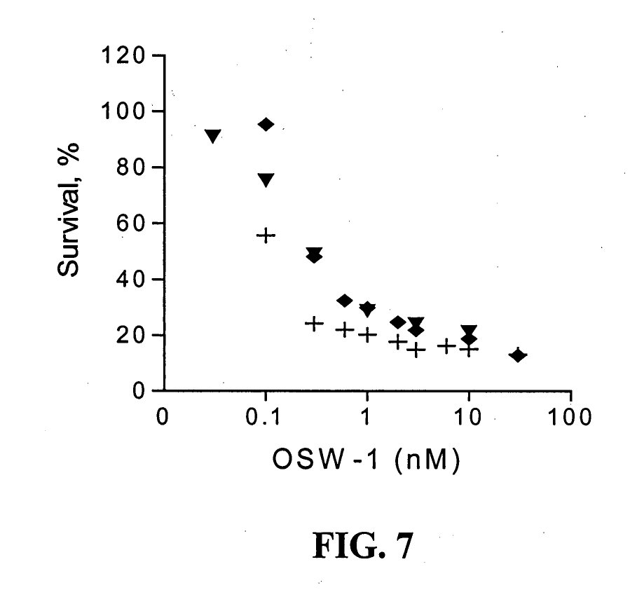

Reduction of Cell Viability in CLL-Patient-Derived Cells. OSW-1 was also found to reduce the viability and cell survival in primary human leukemia cells isolated from patients with chronic lymphocytic leukemia (CLL). Freshly isolated primary CLL cells were incubated with the 0-100 nM OSW-1 for 72 hours in vitro and cell viability was measured by the MTT assay. The IC₅₀ value was found to be 0.15±0.26 nM (n=23 patients) see data depicted in FIG. 7.

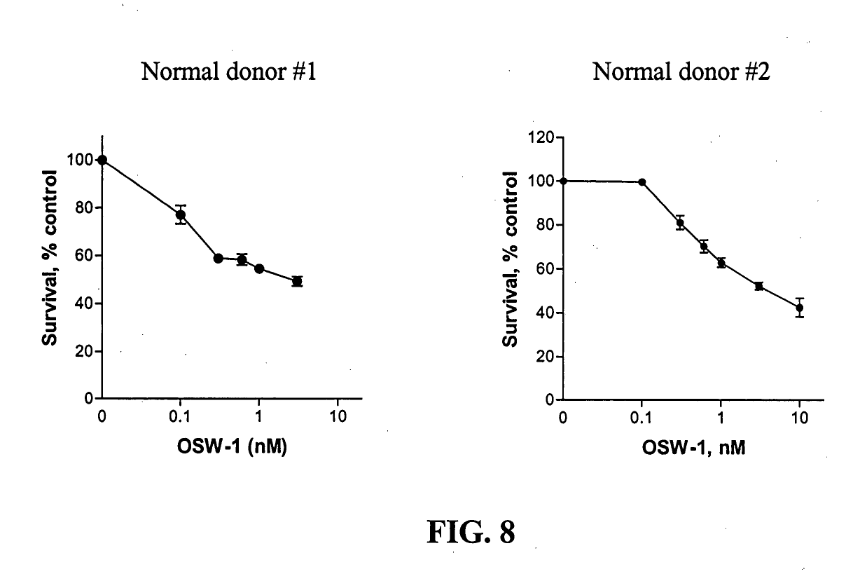

As controls the effect of OSW-1 on cell survival in primary normal lymphocytes isolated from healthy donors was also analyzed. Freshly isolated normal lymphocytes were incubated with the 01-0 nM OSW-1 for 72 hours in vitro and cell viability was measured by MTT assay. As depicted in FIG. 8, the IC₅₀ value estimated to be 4 nM and 3 nM in case #1 and #2, respectively.

A comparison of cytotoxic effect of OSW-1 in primary human leukemia (CLL) cells and primary normal lymphocytes is also presented in Table 2. The selectivity index was calculated as the IC₅₀ ratio of malignant/normal cells.

TABLE 2

| Cell Type | IC₅₀ |
|---|---|
| CLL leukemia cells | 0.15 ± 0.26 nM |
| Normal Lymphocytes | 3.5 ± 0.7 nM |
| Selectivity Index | 23 |

### Example 3

### Role of Mitochondrial Respiration

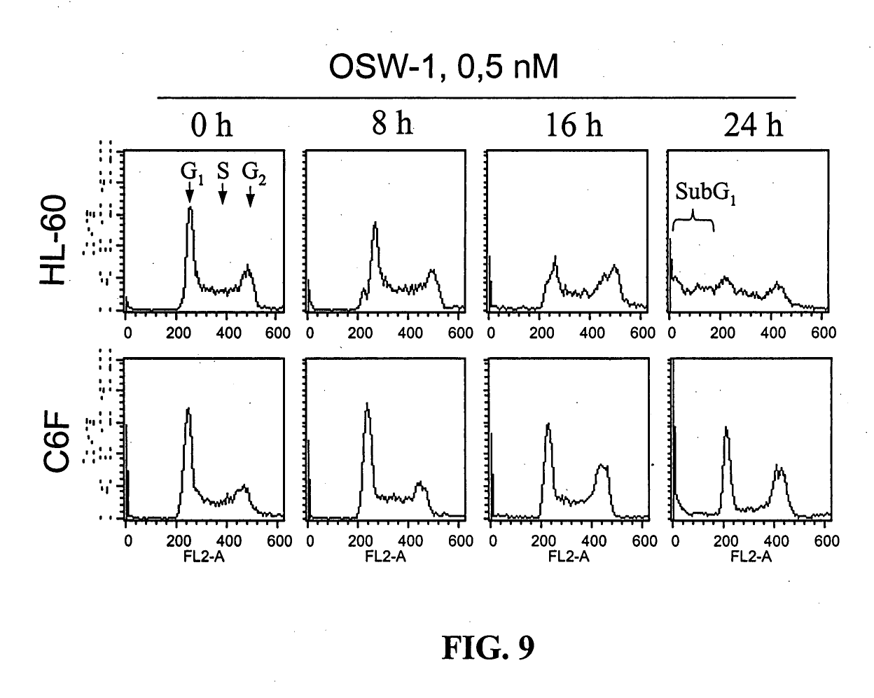

Mitochondrial respiration was found to have an important role in the cytotoxicity of OSW-1. Two cancer cell lines and their mutant mitochondrial respiration deficient clones were analyzed for apoptosis and changes in cell-cycle following exposure to OSW-1. Thus, the parental HL-60 leukemia line and its mitochondrial respiration defective mutant C6F were incubated with 0.5 nM OSW-1 for 0, 8, 16 and 24 hours and apoptosis and change in cell cycle distribution were assayed by flow cytometry analysis. The data is depicted in FIG. 9 and demonstrates that the respiration defective cells are resistant to the effects of OSW-1. FIG. 9 demonstrates that the parental HL-60 cells (respiration-competent) exhibit a significant change in cell cycle distribution 16 hours after incubation with OSW-1, as evidenced by a significant reduction of G1 phase cells and an increase of cells in the G2/M phase region. By 24 h, a substantial portion of the drug-treated HL-60 cells became apoptotic, and appeared as subG₁ cells, which reflect a loss of cellular DNA during apoptosis as the consequence of OSW-1 treatment (FIG. 9, upper panels). In contrast, treatment of the respiration-deficient C6F cells with OSW-1 under identical conditions caused only a slight increase of cells in G2/M phase at the later time points (16 and 24 h), and does not result in significant apoptosis (FIG. 9, lower panels). Thus, it is clear that the respiration defective cells are resistant to the effects of OSW-1. Consistent with these observations, apoptosis was also detected by DNA fragmentation assays performed at 24 and 32 hours after HL-60 cells are treated with OSW-1; DNA fragmentation was not observed in C6F cells even at 32 hours of drug incubation (data not shown).

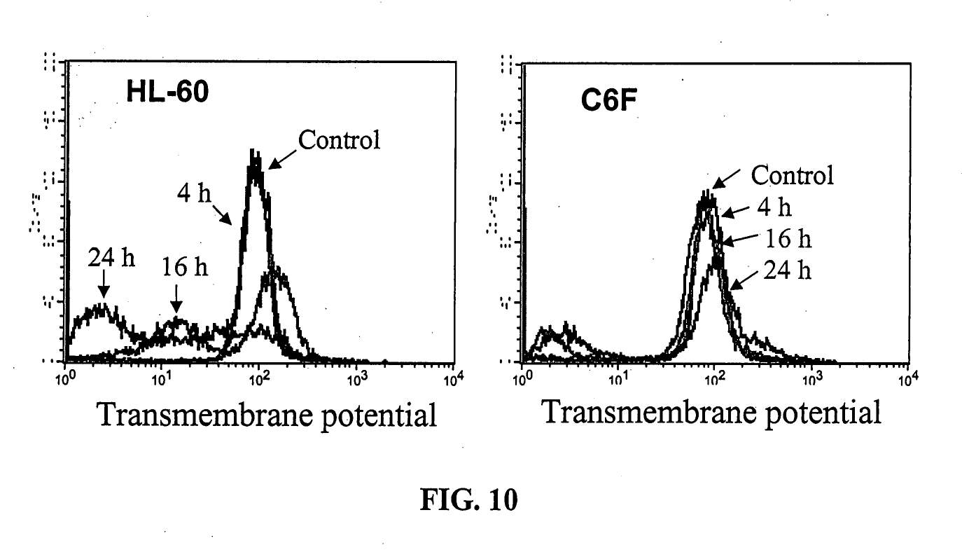

The effect of OSW-1 on mitochondrial transmembrane potential in HL-60 cells and C6F cells was also analyzed by incubating the cells with 0.5 nM OSW-1 for the 4, 16 and 24 hours. Change in mitochondrial transmembrane potential was measured by flow cytometry analysis, using rhodamine-123 as a potential-sensitive fluorescent dye and are depicted in FIG. 10. Treatment of the respiration-competent HL-60 cells with orsaponin did not cause a significant change in mitochondrial transmembrane potential at the early time point (4 h). However, a prolonged incubation (16-24 h) resulted in a substantial loss of mitochondrial transmembrane potential in HL-60 cells, as evidenced by the decrease of the peak at the normal region with relative fluorescent intensity of 100 units, and the appearance of a new peak on the left with lower fluorescent intensity (FIG. 10). In contrast, the respiration-deficient C6F cells treated with the same concentration of OSW-1 did not show significant loss of mitochondrial transmembrane potential (FIG. 10). This is consistent with the observations shown in FIG. 9, and indicates that mitochondrial respiration may plays an important role in the cytotoxicity of OSW-1.

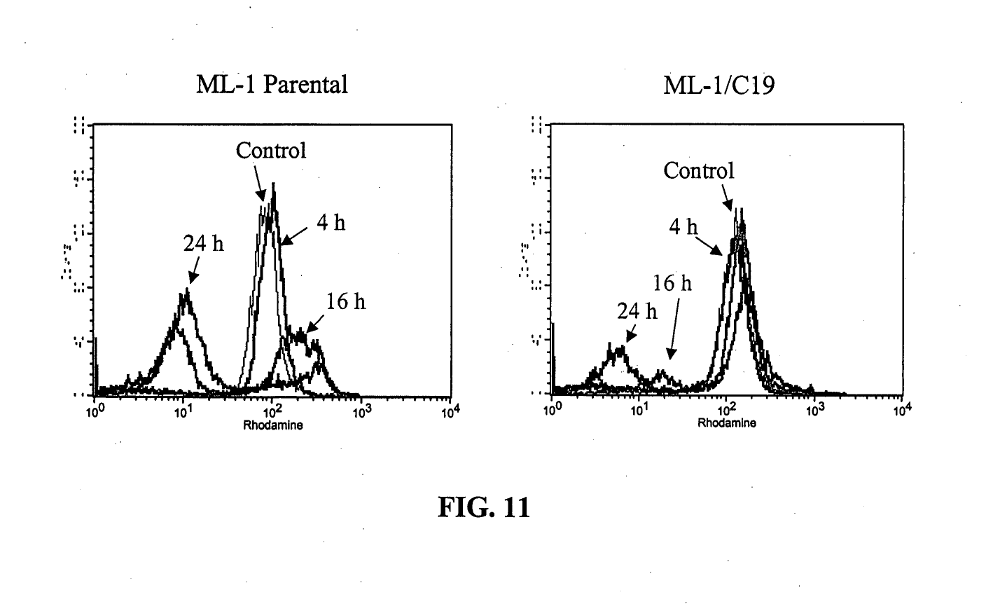

The effect of OSW-1 on mitochondrial transmembrane potential of ML-1 cells and the corresponding mitochondrial respiration defective mutant cell line C19 were analyzed by incubating with 1 nM OSW-1 for 4, 16 and 24 hours. Change in mitochondrial transmembrane potential was measured by cytometry analysis, using rhodamine-123 as a potential-sensitive fluorescent probe and are depicted in FIG. 11. In vitro incubation of the respiration-competent ML-1 cells with orsaponin cause a substantial loss of mitochondrial transmembrane potential in ML-1 cells at 16 and 24 h, as evidenced by the decrease of the peak at the region with normal fluorescent intensity, and the appearance of a new peak on the left with much low fluorescent intensity (FIG. 11). In contrast, treatment of the respiration-deficient C19 cells with the same concentration of OSW-1 (1 nM) caused a loss of mitochondrial transmembrane potential in only a small portion the cells (FIG. 11). These data show that mitochondrial respiration plays an important role in the cytotoxicity of OSW-1.

### Example 4

### Effects on Gene Expression In Pancreatic Cancer Cells

Effect of OSW-1 on gene expression in the pancreatic cancer cell line AsPc-1 was analyzed by DNA microarray analysis. Cells were incubated with 0.3 nM OSW-1 for 14 h as apoptosis occurred at 16-20 h in these cells, and DNA microarray analysis was performed by methods known in the art.

The micorarray is a glass-slide-based cDNA array containing 1100 known genes in duplicate grouped according to their known functions in various signal transduction pathways, including apoptosis, DNA damage and repair, cell cycle, and mitochondrial related genes. RNA was isolated from the control AsPC-1 cells and from cells treated with OSW-1 (0.3 nM, 14 h). The RNA samples were converted by reversed transcription to cDNA, which was labeled with red fluorescent dye (control) or green fluorescent dye (OSW-1 treated). After calibration for fluorescent intensity, appropriate amount of each labeled sample was added onto the microarray slide for competing hybridization. The signals on the array were collected by a fluorescent microscanner. A red spot indicates a higher expression of that particular gene in the control cells (an indication of decreased expression in the OSW-1-treated cells), whereas a green spot reflects an increased gene expression in the OSW-1-treated cells. A yellow spot shows equal expressions in both samples. The fluorescent intensity serves as a quantitative index of relative gene expression levels. The data were analyzed using appropriate software. Table 3 below has a list of genes whose expression changed significantly after treatment with OSW-1. A close examination of the gene expression profile in OSW-1-treated cells revealed that many of the genes that showed a significant change in expression after treatment with OSW-1 are molecules that are involved in mitochondrial respiration. These genes include NADH dehyrogenase, ubiquinol-cytochrome c reductase, and cytochrome c oxidase. These findings again suggest that the action of OSW-1 may involve mitochondrial respiration.

TABLE 3

| Increased Expression | Decreased Expression |
|---|---|
| Cytochrome c oxidase VIa | Zinc finger protein 205 |
| NADH dehyrogenase 1β-1 | Zinc finger protein 85 |
| Dipeptidylpeptidase IV | UQ-Cyt C reductase hinge protein |
| Cytochrome c oxidase VIIb | Cadherin 17 |
| NADH dehyrogenase 1β-4 | DNA pol α |
| RNA adenosine deaminase | Calcium signal transducer-2 |
| Cytochrome c oxidase IV | MAPK kinase |
| NADH Dehyrogenase 1α-7 | Insulin-like GF-2 receptor |
| HMG Protein isoform I | CD48 antigen |

### Example 5

### Mouse Models Of Cancer

In an initial round of in vivo trials, the inventors used mouse models of human cancer with the histologic features and metastatic potential resembling tumors seen in humans and treated these animals with the orsaponin compositions to examine the in vivo efficacy of the orsaponins in terms of suppression of tumor development.

Thus, nude mice were inoculated with human ovarian cancer SKOV3 cells at a concentration of 2×10⁶/mouse, by the intraperitoneal route (i.p.) with 10 mice/group. Treatment with OSW-1 was started on day 6 after tumor inoculation. The control group was treated with saline. OSW-1 was given by i.p. injection, 10 μg/kg/day, 5 days/week for two weeks. The mice were observed for survival without any further drug treatment. When severe tumor burden and moribund signs appeared, euthanasia for the affected animal was performed according to IACUC standards.

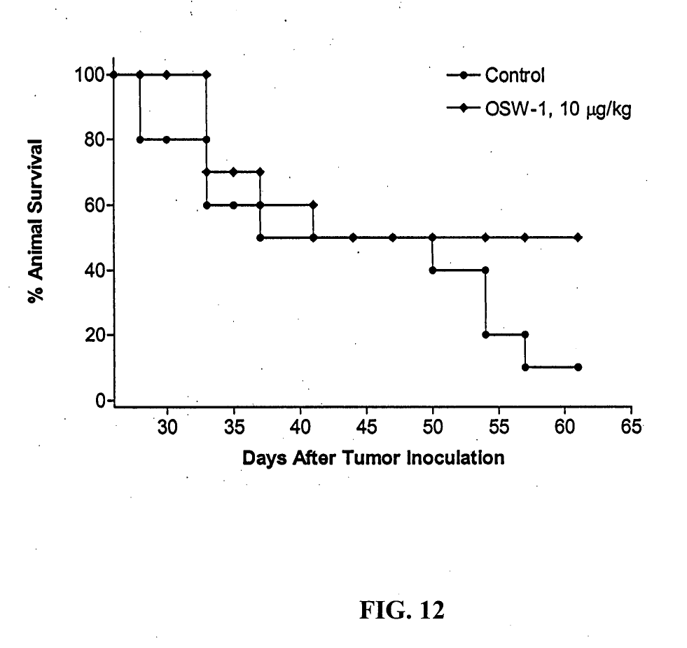

The results of the study are depicted in FIG. 12 and summarized in Table 4 below. Clearly, the mice treated with OSW-1 had better survival due to reduced tumor burden.

TABLE 4

Survival %

| Animal Group | 30 days | 60 days |
|---|---|---|
| Untreated | 80% | 10% |
| OSW-1 | 100% | 50% |

As is well known, the nude mouse has been used in experimental and clinical research since it was first described in 1969 (Rygaard and Povlsen, 1969). It is generally accepted that the nude mouse model is the best indication of what can be expected from human trials. There are numerous studies that support that transplants of human tumors into the nude mouse provide an accepted model for testing the clinical efficacy of anticancer agents (Inoue et al., 1983; Guiliani et al., 1981; Giovanella et al., 1983; Tashiro et al., 1989, Khleif and Curt, 1997, each incorporated herein by reference). Positive results from nude mouse studies indicate a reasonable expectation of positive results in clinical trials. The nude mouse has also been used to screen for, study and confirm anticancer effects of numerous agents. Literature supports the concept that doses of compounds used in preclinical animal studies can be correlated to studies in human clinical trials (Tashiro et al., 1989). Correlation between the nude mouse and human clinical responses to, for example, cyclophosphamide, 1-(4-amino-2-methylpyrimidin-5-yl)-methyl-3-(2-chloroethyl)-3-nitrosurea hydrochloride, vinblastine and 5-fluorouracil have been shown. Further, other studies used BALB/c nude mouse model for human breast cancer to evaluate the antitumor activity of a variety of drugs, including vincristine, vinblastine, vindesine, dauonomycin, mitoxantrone, and 5-fluorouracil amongst others (Inoue et al., 1983). These studies showed good correlation between anticancer activity of various drugs in the nude mouse model for human breast cancer and in clinical treatment in humans. In yet another comprehensive study (Guiliani et al., 1981), BALB/c nude mice were transplanted with breast, colon, lung, melanoma, ovarian prostate and larynx cancers and the effects of doxorubicin on these cancer models was studied. It was found that in each case the results from the model studies correlated extremely well with clinical data. The National Cancer Institute has even employed a development scheme in assaying for in vivo antitumor activity in which the human tumor cell line most sensitive to an active candidate in vitro is tested as a xenograft in a subcutaneous implant site in a nude mouse (Cancer: Principles & Practice of Oncology, 5th Ed., 1997, pp. 392-94).

In addition to the use of nude mice models of cancer, one can also use severe combined immunodeficiency (SCID) mice for transplantation of normal and malignant human cells (Flavell, 1996). The SCID mouse model has also been employed in the art to predict therapeutic benefits of antitumor therapy in SCID mice bearing human leukemias and lymphomas (Flavell, 1996). Similar studies using nude or SCID mice models of other cancer types, including pancreatic cancers and other solid cancers and treating them with OSW-1 compositions are contemplated.

Thus, the above studies demonstrate that the mouse model emulates the clinical situation in of ovarian cancer and shows the efficacy of OSW-1.

### Example 6

### Clinical Trials

This example is concerned with the development of human treatment protocols for anticancer therapy for pancreatic cancers, colon cancers, ovarian cancers or CLL's using the orsaponin therapy either alone or in combination with other therapeutic agents. One of skill in the art will also recognize that any other adjunct cancer therapy known is contemplated as useful as a second anti-cancer agent in combination or conjunction with the present therapeutic methods.

The various elements of conducting a clinical trial, including patient treatment and monitoring, are known to those of skill in the art and in light of the present disclosure. The following information is being presented as a general guideline for use in establishing the therapies using the anticancer orsaponin comprising compositions described herein alone or in combinations with other adjunct treatments used routinely in cancer therapy in clinical trials. Any clinical trials, of course, must be approved by the appropriate authorities such as IRB and FDA.

Candidates for the phase 1 clinical trial will be patients with pancreatic cancer, CLL, colon cancers, or ovarian cancers on which all conventional therapies have failed. Approximately 100 patients will be treated initially. Their age will range from 16 to 90 (median 65) years. Patients will be treated, and samples obtained, without bias to sex, race, or ethnic group. For this patient population of approximately 41% will be women, 6% will be black, 13% Hispanic, and 3% other minorities. These estimates are based on consecutive cases seen at MD Anderson Cancer Center over the last 5 years.

Optimally the patient will exhibit adequate bone marrow function (defined as peripheral absolute granulocyte count of >2,000/mm³ and platelet count of 100, 000/mm³, adequate liver function (bilirubin 1.5 mg/dl) and adequate renal function (creatinine 1.5 mg/dl).

Research samples will be obtained from peripheral blood or marrow under existing approved projects and protocols. Some of the research material will be obtained from specimens taken as part of patient care.

The therapy with compositions comprising the orsaponins described herein will be administered to the patients regionally, systemically or locally on a tentative weekly basis. A typical treatment course may comprise about six doses delivered over a 7 to 21 day period. Upon election by the clinician the regimen may be continued with six doses every three weeks or on a less frequent (monthly, bimonthly, quarterly, etc.,) basis. Of course, these are only exemplary times for treatment, and the skilled practitioner will readily recognize that many other time-courses are possible.

The modes of administration may be local administration, including, by intratumoral injection and/or by injection into tumor vasculature, intratracheal, endoscopic, subcutaneous, and/or percutaneous. The mode of administration may be systemic, including, intravenous, intra-arterial, intra-peritoneal and/or oral administration.

The orsaponin compositions will be administered at appropriate dosages as determined by a trained physician by a suitable route as discussed above. Dosage ranges of 0.5-50 μg/kg are contemplated as useful. Of course, the skilled artisan will understand that while these dosage ranges provide useful guidelines appropriate adjustments in the dosage depending on the needs of an individual patient factoring in disease, gender, age and other general health conditions will be made at the time of administration to a patient by a trained physician. The same is true for means of administration, routes of administration as well.

To monitor disease course and evaluate the cancer cell killing it is contemplated that the patients should be examined for appropriate tests periodically. To assess the effectiveness of the drug, the physician will determine parameters to be monitored depending on the type of cancer/tumor and will involve methods to monitor reduction in tumor mass by for example computer tomography (CT) scans, detection of the induction of cell death in the tumor, and in some cases the additional detection of other tumor markers such as CA 19.9, CEA, TPA and CA 242, which are markers of pancreatic cancer or tissue polypeptide specific antigen (TPS) and carbohydrate antigen 125 (CA-125) which are markers of ovarian cancers, or multiple myeloma-1/interferon regulatory factor-4 (MUM 1/IRF4), cyclin D, CD38, or other markers of CLL may be analyzed. Tests that will be used to monitor the progress of the patients and the effectiveness of the treatments include: physical exam, X-ray, blood work, bone marrow work and other clinical laboratory methodologies. The doses given in the phase 1 study will be escalated as is done in standard phase 1 clinical phase trials, i.e., doses will be escalated until maximal tolerable ranges are reached.

Clinical responses may be defined by acceptable measure. For example, a complete response may be defined by complete disappearance of the cancer cells whereas a partial response may be defined by a 50% reduction of cancer cells or tumor burden.

The typical course of treatment will vary depending upon the individual patient and disease being treated in ways known to those of skill in the art. For example, a patient with pancreatic adenocarcinoma might be treated in four week cycles, although longer duration may be used if no adverse effects are observed with the patient, and shorter terms of treatment may result if the patient does have side effects.

### Example 7

### Toxicity of Orsaponin in Mice

To assess the toxicity of orsaponin of the present invention, a 5-day intravenous study with Orsaponin in mice was conducted. Male and female ICR mice, 15 per group, were injected intravenously with 10, 20 or 40 μg/kg/day of Orsaponin (Table 5). Five mice from each group were scheduled to be sacrificed 3, 14, and 42 days after the last dose was administered.

TABLE 5
Group Designation for OSW1

| | | Dosage Level |
|---|---|---|
| Group Designation | Number/Group | (μg/kg/day) |
| 1 | 15/Sex | 0 |
| 2 | 15/Sex | 10 |
| 3 | 15/Sex | 20 |
| 4 | 15/Sex | 40 |

Tissues (liver with gall bladder, kidneys, heart with aorta, lungs, spleen, skeletal muscle, brain, pituitary gland, eyes, pancreas, stomach, duodenum, jejunum, ileum, cecum, colon, mesenteric lymph node, salivary gland, mandibular lymph node, thymus, adrenal glands, larynx/pharynx/tongue, thyroid gland, parathyroid, trachea, esophagus, skin, mammary gland (females only), prostate, seminal vesicles, urinary bladder, testes, epididymides, ovary, uterus, cervix, femur/knee joint, sternum, bone marrow, and gross lesions) were processed for microscopic evaluation.

The results showed striking difference in susceptibility to Orsaponin toxicity between the genders. In female mice, no mortality or morbidity was observed. No Orsaponin-related findings were observed in female mice at any of the doses administered.

On the other hand, in male mice mortality and morbidity was observed in the 20 and 40 μg/kg/day male mice after the fourth injection. The 40 μg/kg/day group was terminated early on study day 4.

TABLE 6
Morbidity/Mortality Summary

| | Sex | | | | | | | | |
|---|---|---|---|---|---|---|---|---|---|
| | Males | | | | Females | | | | |
| Dose (μg/kg/day) | 0 | 10 | 20 | 40 | 0 | 10 | 20 | 30 | 40 |
| Total | 15 | 15 | 15 | 15 | 15 | 15 | 15 | 15 | 15 |
| Dosed | | | | | | | | | |
| Early | 0 | 0 | 7 | 15 | 0 | 0 | 0 | 0 | 0 |
| Deaths | | | | | | | | | |

No hematology was done on the male mice sacrificed in extremis. What blood was collected was used for clinical chemistries (Table 7). Results from male mice in the 30 and 40 μg/kg/day groups sacrificed in extremis on day 4 are included in Table 7 below. The AST, ALT, and total bilirubin indicate severe liver disease.

TABLE 7
Clinical Chemistry

| MALE MOUSE | Hemolysis | Comments | T. BILI | SODIUM | POTASSIUM | CHLORIDE | CREATININE |
|---|---|---|---|---|---|---|---|
| Early Deaths | Sl, Md, Mk | ICT/*UTP | ug/dl | mEq/L | mEq/L | mmol/L | ug/dl |
| OSW1-02-3001 | | ICT | 2.3 | QNS | QNS | 109 | 0.5 |
| OSW1-02-3011 | Md | ICT | 1 | QNS | QNS | QNS | 0.5 |
| OSW1-02-4001 | Md | ICT | 2.6 | QNS | QNS | 107 | 0.3 |
| OSW1-02-4005 | | ICT | 2.2 | QNS | QNS | 103 | 0.5 |
| OSW1-02-4007 | | ICT | 2.8 | QNS | QNS | 110 | 0.4 |
| OSW1-02-4009 | Md | ICT | 2 | QNS | QNS | 116 | 0.1 |
| OSW1-02-4011 | Md | ICT | 1.6 | QNS | QNS | QNS | 0.6 |
| OSW1-02-4013 | Md | ICT | 1.1 | QNS | QNS | 110 | 0.4 |
| OSW1-02-4021 | Md | ICT | 2.3 | QNS | QNS | QNS | 0.6 |
| OSW1-02-4025 | | ICT | 2.7 | QNS | QNS | 105 | 0 |
| OSW1-02-4027 | Mk | ICT/ | 7.6 | QNS | QNS | QNS | 1.5 |
| | | *UTP | | | | | |
| OSW1-02-4029 | Sl | ICT | 2.4 | QNS | QNS | 110 | 0 |

| MALE MOUSE | BUN | PHOSPHORUS | AST | ALT | ALK PHOS | T. PROTEIN | Alb | Glob |
|---|---|---|---|---|---|---|---|---|
| Early Deaths | ug/dl | ug/dl | IU/L | IU/L | IU/L | gm/dl | gm/dl | gm/dl |
| OSW1-02-3001 | 19.9 | 10.4 | 1794 | 1058 | 1896 | 6.5 | 4.2 | 2.3 |
| OSW1-02-3011 | 17.2 | 13.5 | 1167 | 486 | 1746 | 4.8 | 3 | 1.8 |
| OSW1-02-4001 | 23.2 | 10.8 | >8800 | >8800 | 2290 | 10 | 3.4 | 6.6 |
| OSW1-02-4005 | 48 | 14.8 | >8800 | >4400 | 3302 | 3.8 | 2.4 | 1.4 |
| OSW1-02-4007 | 28 | 12.6 | >8800 | >8800 | >4500 | 5.4 | 3.8 | 1.6 |
| OSW1-02-4009 | 16.4 | 11.8 | 3306 | 2060 | >1500 | 5.6 | 3.8 | 1.8 |
| OSW1-02-4011 | 34.8 | 16.8 | >6600 | >6600 | >1500 | 4.8 | 3.3 | 1.5 |
| OSW1-02-4013 | 15.7 | 13.8 | 4341 | 5642 | 2001 | 6 | 3.9 | 2.1 |
| OSW1-02-4021 | 48 | 19.8 | >4400 | >4400 | >2842 | 4 | 2.8 | 1.2 |
| OSW1-02-4025 | 17.4 | 9.6 | 5106 | 4950 | 3339 | 4.1 | 2.7 | 1.4 |
| OSW1-02-4027 | 18.9 | 19.6 | 5864 | 5596 | 4992 | 6 | * | * |
| OSW1-02-4029 | 14.8 | 10.5 | >6600 | >6600 | 4326 | 3.4 | 2.4 | 1 |

Individual animal organ weight was also measured. Calculation of percent of control using the relative weight of the individual organs per 100 grams of the terminal body weight (TBW) is included below in Tables 8 and 9. A change of greater than 20% difference was considered toxicologically important, and/or a clear dose response relationship.

TABLE 8
Female Average Organ Weight Data
Expressed as a Percent of Control

| | Percent of Control Organ Weight Data: Females | | | |
|---|---|---|---|---|
| | Relative Weight | | | |
| μg/kg/day | TBW | Liver | Kidney | Spleen |

3-Day (avg)

| | | | | |
|---|---|---|---|---|
| 0 | 100 | 100 | 100 | 100 |
| 10 | 101 | 92 | 96 | 87 |
| 20 | 99 | 88 | 94 | 93 |
| 40 | 91 | 84 | 90 | 93 |

14-Day (avg)

| | | | | |
|---|---|---|---|---|
| 0 | 100 | 100 | 100 | 100 |
| 10 | 112 | 106 | 102 | 92 |
| 20 | 118 | 102 | 104 | 109 |
| 40 | 118 | 105 | 130 | 101 |

42-Day (avg)

| | | | | |
|---|---|---|---|---|
| 0 | 100 | 100 | 100 | 100 |
| 10 | 91 | 93 | 85 | 101 |
| 20 | 89 | 97 | 116 | 106 |
| 40 | 93 | 92 | 105 | 100 |

No dose-related alterations in organ weights were observed in female mice. The 30% increase in the relative kidneys weight in the 40 μg/kg/day group at the 2 week sacrifice was due to a single animal having hydronephrosis, and was not compound related.

TABLE 9
Male Average Organ Weight Data Expressed
as a Percent of Control

Percent of Control Organ Weight Data: Males
Relative Weight

| | | | | |
|---|---|---|---|---|
| μg/kg/day | TBW | Liver | Kidney | Spleen |

3-Day (avg)

| | | | | |
|---|---|---|---|---|
| 0 | 100 | 100 | 100 | 100 |
| 10 | 91 | 110 | 103 | 125 |
| 20 | 76 | 111 | 116 | 66 |
| 40 | 80 | 114 | 69 | 73 |

14-Day (avg)

| | | | | |
|---|---|---|---|---|
| 0 | 100 | 100 | 100 | 100 |
| 10 | 107 | 113 | 101 | 126 |
| 20 | 106 | 125 | 110 | 136 |

42-Day (avg)

| | | | | |
|---|---|---|---|---|
| 0 | 100 | 100 | 100 | 100 |
| 10 | 128 | 100 | 93 | 111 |
| 20 | 117 | 118 | 100 | 82 |

Male mice in the 20 and 40 μg/kg/day groups had terminal body weight that were 34 and 20% of their respective group average of controls in the 3 day sacrifice. The relative liver weight in these groups was 111 and 114% of the control relative liver weight. This increase in liver weight in a severely decreased body weight correlates with hepatocellular hypertrophy. The relative average weight of the spleen was 66 and 73% of control spleen weights in the 20 and 40 μg/kg/day groups, respectively. This decrease in spleen weight correlated with necrosis/apoptosis of the lymphocytes observed microscopically.

At the approximate 2-week recovery sacrifice, the average relative organ weight of the spleen was slightly elevated correlating with the recovery of lymphocytes in the spleen.

Mice from the 20 and 40 μg/kg/day groups were examined for lesions. No gross lesions were observed in female mice. However, in male mice, Orsaponin-related lesions were observed in the liver, lymphoid tissues, gastrointestinal tract and testes (Table 10). The livers were pale and/or spotty, and the gastrointestinal tract contained hemorrhage. The mice in the 20 μg/kg/day males were designated as the high dose group, and surviving mice were designated the recovery animals and scheduled at 2 and 6 weeks after dosing for sacrifice.

TABLE 10
Orsaponin-Related Microscopic Observations
in Male Mice Through Day 8

| | Dose μg/kg/day | | | |
|---|---|---|---|---|
| Tissue/Lesions | 0 | 10 | 20 | 40 |
| Liver (# examined) | 5 | 5 | 6 | 15 |
| Necrosis, hepatocellular, periportal | | | 3 | 15 |
| Hypertrophy, hepatocellular, panlobular | | 1 | 4 | 15 |
| Biliary Hyperplasia | | | 3 | 9 |
| Edema, gall bladder | | | 2 | 2 |
| Hemorrhage | | | 1 | 9 |
| Lymphoid Tissues | | | | |
| Spleen (# examined) | 5 | 5 | 6 | 15 |
| Lymphoid necrosis/trophy | | | 5 | 15 |
| Congestion | | | 2 | 5 |
| Thymus (# examined) | 5 | 5 | 2 | 13 |
| Lymphoid necrosis/atrophy | | | 1 | 12 |
| Mesenteric Lymph Node* (# examined) | 4 | 5 | 6 | 14 |
| Lymphoid necrosis/atrophy | | | 3 | 12 |
| Testes (# examined) | 5 | 4 | 6 | 14 |
| Seminiferous tubule degeneration/atrophy | | | 1 | 3 |
| Epididymides (# examined) | 5 | 4 | 6 | 15 |
| Oligospermia | | | 1 | 3 |
| Gastrointestinal Hemorrhage | | | 4 | 6 |
| (from gross tables, # of animals) | | | | |

In the 20 and 40 μg/kg/day groups, significant toxic lesions in the liver were due to necrosis and biliary hyperplasia; hemorrhage, and gall bladder edema were also observed. Hepatocellular hyperptrophy occurred in all dose groups but with much less incidence in the 10 μg/kg/day group. The hepatocellular lesion was completely reversible at 2 and 6 weeks.

The lymphoid necrosis, testicular atrophy, and gastrointestinal hemorrhage were observed in the 20 and 40 μg/kg/day groups only, and all were reversed within 2 and 6 weeks. The lymphoid atrophy in the thymus did not recover, but the splenic lymphoid tissues did recover. The liver lesion consisted of hepatocellular necrosis, hepatocellular hypertrophy, biliary hyperplasia, and hemorrhage. Systemic lymphoid necrosis/atrophy was observed in the spleen, lymph nodes, thymus, and gut associated lymphoid tissue. The gastrointestinal tract had hemorrhage into the lumen and submucosal tissues in the 20 and 40 μg/kg/day groups. Testicular degeneration with oligospermia was observed in males treated with 40 μg/kg/day. The hepatic and testicular lesion was recovered in the 20 μg/kg/day group males surviving to the 2 and 6 week recovery phases. Lymphoid necrosis/apoptosis was recovered 2 and 6 weeks after dosing, but some atrophy remained. In these studies, the males appeared to be the more sensitive gender.

The gender difference in susceptibility to Orsaponin toxicity is striking. In males, the liver is the organ most significantly affected by Orsaponin and liver failure appears to be responsible for the high incidence of early morbidity and mortality. However, the liver injury was recoverable within 2 weeks after the last dose in males treated with 20 μg/kg/day. The lymphoid necrosis observed in the animals from the 3-day sacrificed was recovered at 2 and 6 weeks, but the atrophy persisted. Degeneration of testicular seminiferous tubules and gastrointestinal hemorrhage occurred less often than liver and lymphoid lesions and exhibited recovery in the animals sacrificed and examined at the later times. Although cell death is a prominent manifestation of Orsaponin toxicity in liver, lymphoid tissues, and to a lesser extent seminiferous tubules, obvious cytotoxicity was not observed in gut and bone marrow. The hemorrhage in the gut may be secondary to the hepatoxicity, or may represent a coagulopathy. The observation of the hemorrhage only in animals with severe hepatotoxicity indicates it may be secondary to the liver lesion.

Photoreceptor atrophy of the retina was observed in both male and female mice. The incidence is tabulated in text table 11. This lesion occurred across both sexes and controls and was interpreted as environmentally induced from the lighting in the room or genetically inherited. Both etiologies are reported, and morphological differentiation is not possible (Greaves, 2000). In albino rats and mice, as little as 24 hrs of ‘normal’ room illumination can cause photoreceptor damage (this is reversible even after a few days of exposure as long as the inner segment remains intact); several days of continuous light can lead to permanent degenerative changes. Measurement of the lighting in the room where these animals were housed during this study was determined to be higher than normal lending support that this is light induced.

TABLE 11
Incidence of Photoreceptor Cell
Atrophy in Male and Female Mice

| Incidence of retinal Photoreceptor Cell | | | |
|---|---|---|---|
| Atrophy Observed Microscopically | | | |
| Dose μg/kg/day | | | |
| 0 | 10 | 20 | 40 |

Males

| | | | | |
|---|---|---|---|---|
| 3 days | 3 | NP | 3 | 7 |
| (# examined: 4, 05, 12) | | | | |
| 2 weeks | 1 | NP | NP | NP |
| (# examined: 5, 0, 0, 0) | | | | |
| 6 weeks | 2 | NP | NP | 4 |
| (# examined: (4, 0, 0, 4) | | | | |

Females

| | | | | |
|---|---|---|---|---|
| 3 days | 2 | NP | NP | 5 |
| (# examined: 5, 0, 0, 5) | | | | |
| 2 weeks | 5 | NP | NP | 2 |
| (# examined: 5, 0, 0, 5) | | | | |
| 6 weeks | 2 | NP | NP | 1 |
| (# examined: (4, 0, 0, 2) | | | | |

NP - eyes not processed

The other microscopic findings noted in this study were considered spontaneous and/or incidental, as they were routinely seen in control mice by experienced toxicologic pathologists.

Under the conditions of the study, the no-observed-effect level (NOEL) and the no-observed-adverse-effect level (NOAEL) for females is 40 μg/kg/day, the highest dose tested. No NOEL for male mice was observed. The NOAEL for males was 10 μg/kg/day administered in 5 daily doses based on pathology.

### Example 8

### Synthesis of 17-deoxyorsaponin

Several derivatives of orsaponin were synthesized and their anticancer activities tested. Among the derivatives tested, 17-deoxyorsaponin exhibited potent activity against a variety of human cancer cell lines and primary leukemia cells isolated from patients.

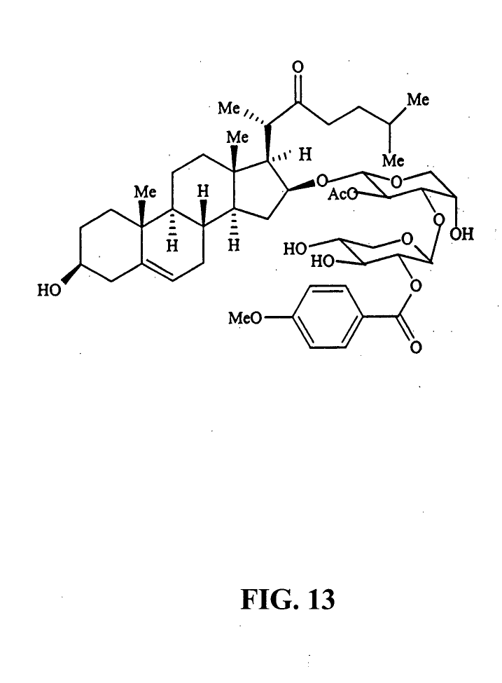

The synthetic scheme for production of 17-deoxyorsaponin is shown below (FIG. 13). Synthesis of the starting material was previously published (Yu et al., 2001; Deng et al., 1999).

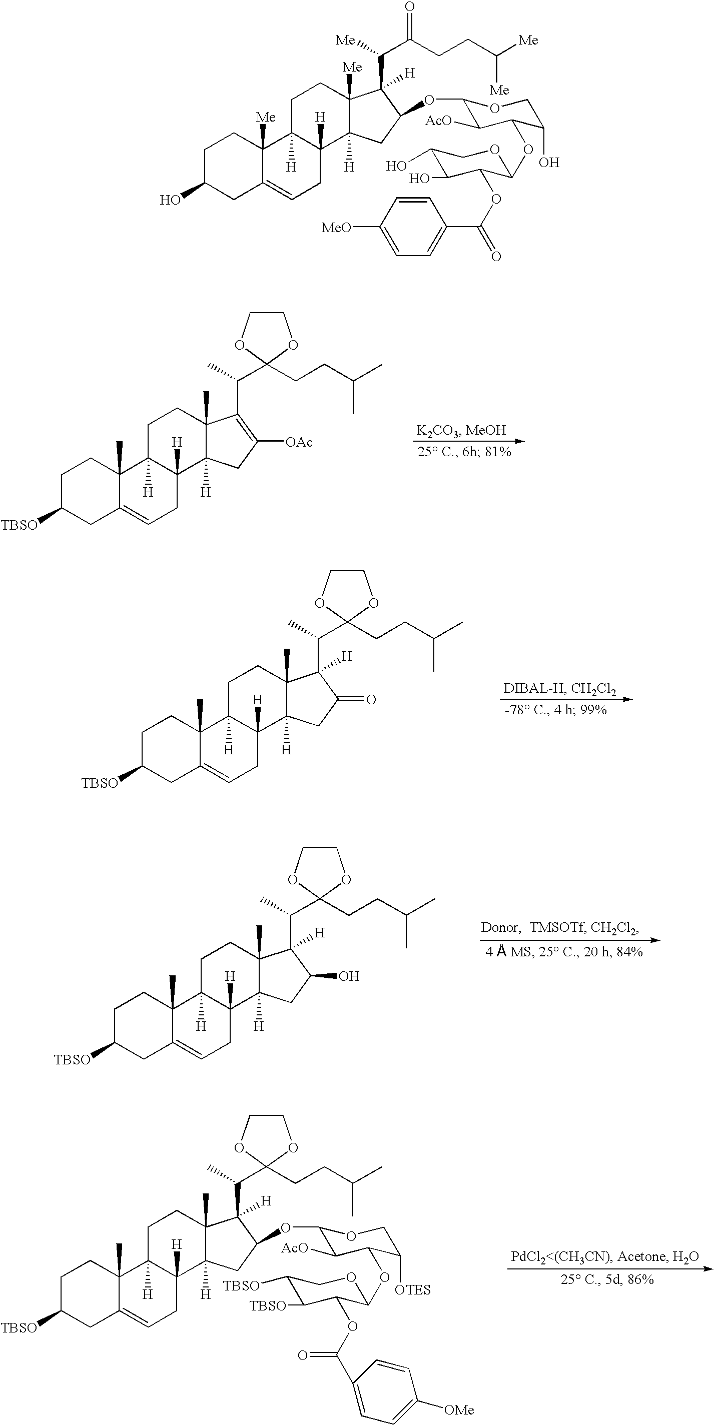

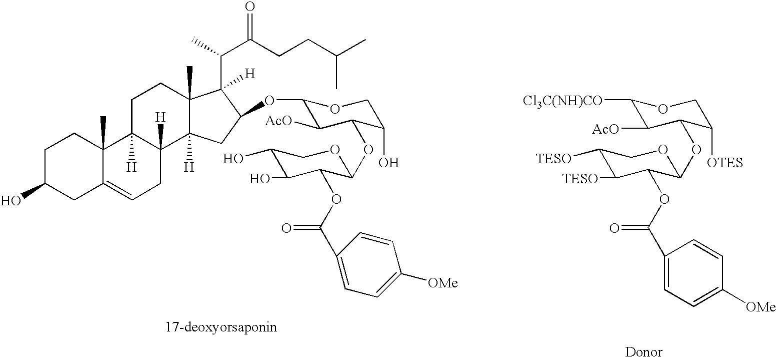

### Example 9

### Anticancer Activity of 17-deoxyorsaponin

The anticancer activity of 17-deoxysaponin was tested in a variety of human cancer cell lines in culture and in primary leukemia cells isolated from patients with chronic lymphacytic leukemia (CLL). Studies were conducted as described in Example 1 above. The cells were treated with various concentrations of 17-deoxysaponin. Growth inhibition was measured using the MMT assay, see Example 1. The results are provided below.

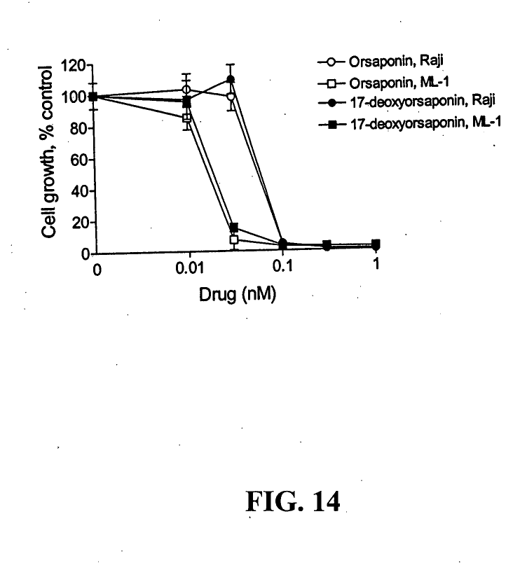

Anticancer activities of Orsaponin and 17-deoxyorsaponin were examined in human leukemia cells (ML-1) and human lymphoma cells (Raji). Cells were incubated with various concentrations of Orsaponin and 17-deoxyorsaponin for 72 h, and cell growth inhibition was measured by MTT assay. Both compounds were found to be effective in inhibiting cancer cell growth with an IC₅₀ value of <0.1 nM (FIG. 14).

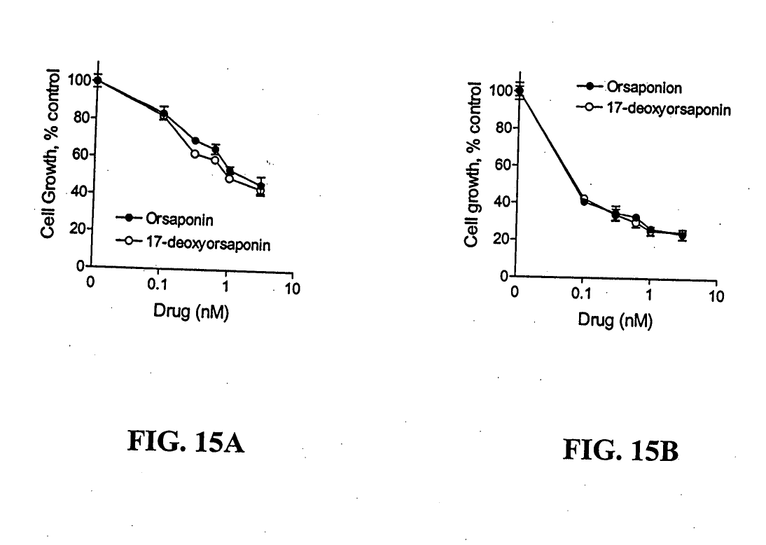

The effect of Orsaponin and 17-deoxyorsaponin in pancreatic cancer cells was also assessed. The human pancreatic cancer AsPC-1 cells and mouse pancreatic cancer (Panco-2) cells were incubated with varying concentrations of the orsaponin compounds, Orsaponin and 17-deoxyorsaponin, for 72 h. Cell growth inhibition was measured by MTT assay. Both compounds exhibited similar cytotoxic activity, with IC₅₀ value of 1 nM for AsPC-1 cells and 0.1 nM for Panco-2 cells (FIGS. 15A-15B).

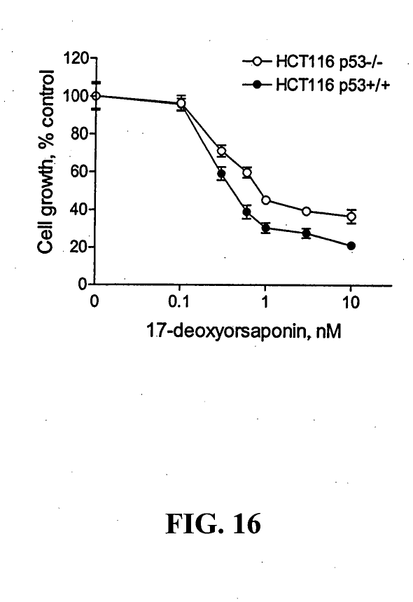

Anticancer activity of 17-deoxyorsaponin in human colon cancer cells was examined. HCT116 p53+/+ and HCT116 p53−/− human colon cancer cells were incubated with the various concentrations of 17-deoxyorsaponin for 72 h. Cell growth inhibition was measured by MTT assay. The IC₅₀ value was found to be approximately 1 nM for both cell lines (FIG. 16). The p53 status of the cells did not significantly affect the antiproliferative activity of the orsaponin compounds.

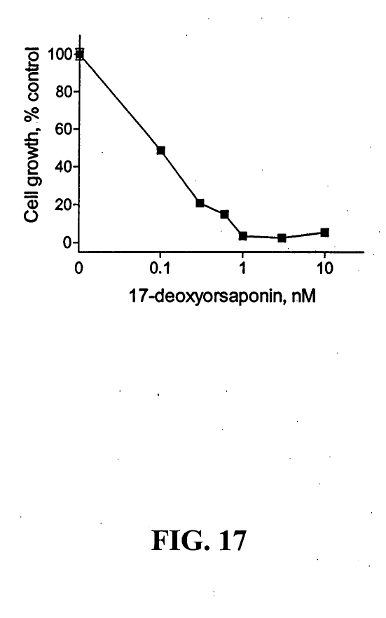

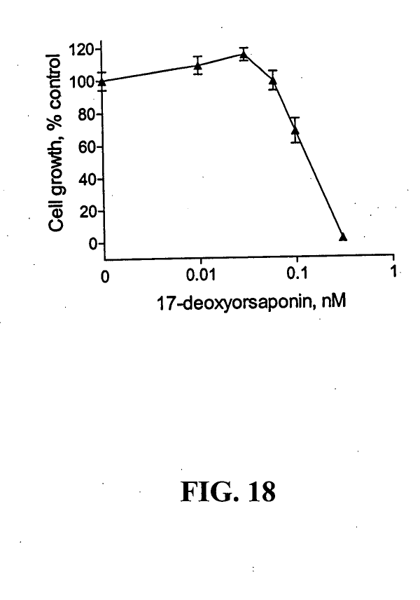

The effect of 17-deoxyorsaponin in human ovarian cancer cells was determined. SKOV3 ovarian cancer cells were incubated with various concentrations of 17-deoxyorsaponin for 72 h. Cell growth inhibition was measured by MTT assay (FIG. 17). This compound was found to be extremely effective in inhibiting the growth of ovarian cancer cells. The IC₅₀ value was found to be of approximately 0.1 nM. The effect of 17-deoxyorsaponin in human acute myeloid leukemia cells was also examined. ML-1 leukemia cells were incubated with various concentrations of 17-deoxyorsaponin for 72 h and cell growth inhibition was measured using the MTT assay (FIG. 18). 17-deoxyorsaponin was found to be very effective in inhibiting the growth of acute leukemia cells with an IC₅₀ value of approximately 0.2 nM.

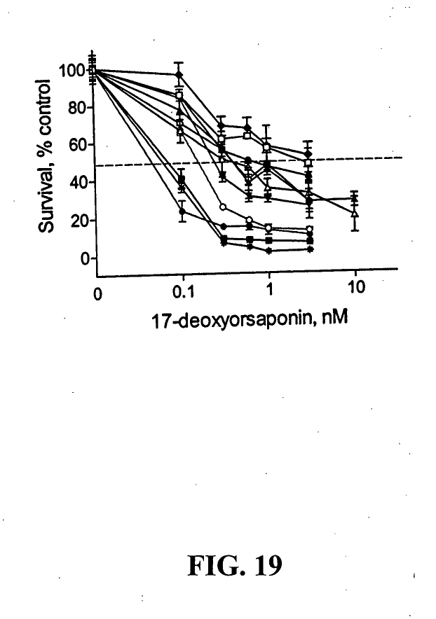

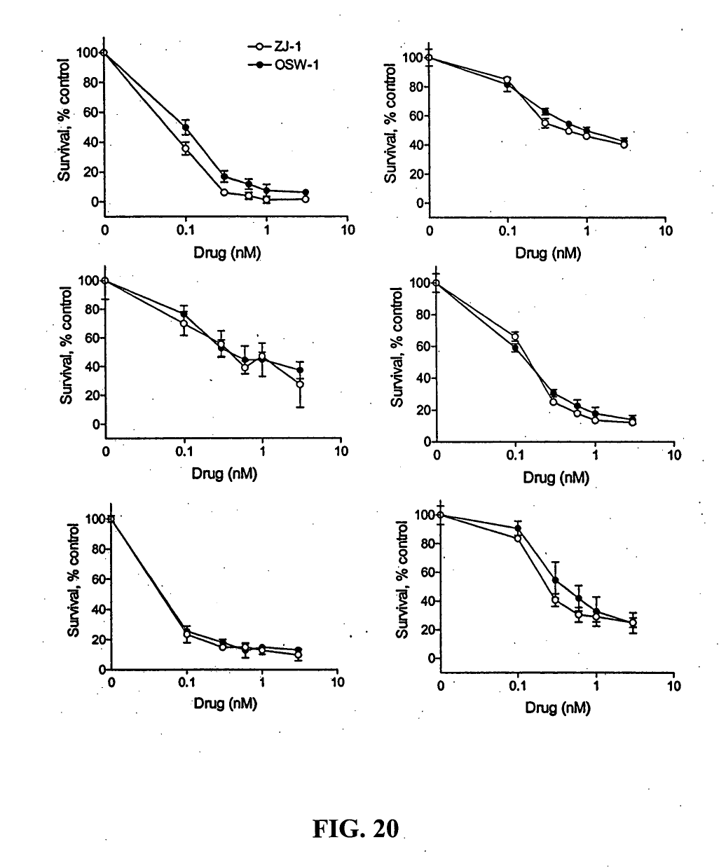

Cytotoxic activity of 17-deoxyorsaponin was also assessed in a number of patient samples. Primary human leukemia cells isolated from 11 patients with chronic lymphocytic leukemia (CLL) were analyzed for cytotoxic activity of 17-deoxyorsaponin. The CLL cells were incubated with various concentrations of 17-deoxyorsaponin for 72h. Cell viability was assayed by MTT assay (FIG. 19). The estimated IC₅₀ value for each patient sample is shown in the Table 12. The median IC₅₀ value is 0.37 nM. Additional studies were conducted to assess the cytotoxic effect of Orsaponin and 17-deoxyorsaponin in primary human leukemia cells isolated from 6 other patients with chronic lymphocytic leukemia. The results show that both compounds have similar potent activity against primary CLL cells in vitro (FIG. 20).

TABLE 12
IC₅₀ of 17-deoxyorsaponin in CLL cells from patient samples

| Patient | IC₅₀ (nM) |
|---|---|
| Pt #1 | <0.1 |
| Pt #2 | <0.1 |
| Pt #3 | <0.1 |
| Pt #4 | 0.12 |
| Pt #5 | 0.34 |
| Pt #6 | 0.37 |
| Pt #7 | 0.46 |
| Pt #8 | 0.57 |
| Pt #9 | 0.83 |
| Pt #10 | 1.81 |
| Pt #11 | 2.28 |
| Median | 0.37 |

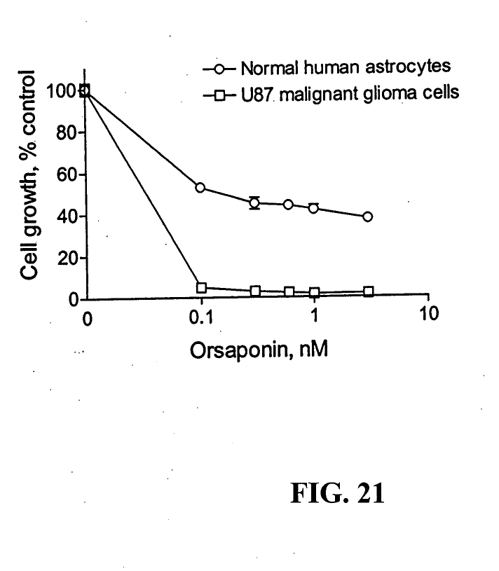

To test the therapeutic selectivity of Orsaponin and 17-deoxyorsaponin, their effect on human brain tumor cells (U87 malignant glioma) and normal human astrocytes were compared. The normal human astrocytes were previously immortalized by transfection with hTER to allow a long-term culture in vitro. The antiproliferative effect of Orsaponin in human malignant glioma cells (U87-MG) and normal human astrocytes was examined. Cells were incubated with various concentrations of Orsaponin for 72 h. Cell growth inhibition was measured by MTT assay (FIG. 21). The results showed that human malignant glioma U87-MG cells are much more sensitive to Orsaponin than normal brain astrocytes (FIG. 21).

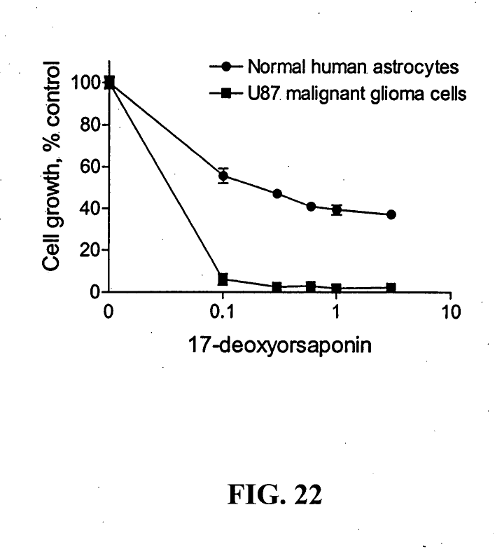

Similarly, brain tumor cells were found to be more sensitive to 7-deoxyorsaponin than normal astrocytes (FIG. 22). The IC₅₀ value of Orsaponin for human malignant glioma U87-MG cells was found to be less than 0.1 nM, whereas the IC₅₀ value for normal human astrocytes was approximately 1 nM. These data suggest the use of Orsaponin and 17-Deoxyorsaponin as therapeutic selectivity agents in treating or preventing human brain cancer.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. patent Ser. No. 10/213,363
Alexakis et al., Tetrahedron Lett., 27:1047, 1986.
Blanchot-Courtois and Hanna, Tetrahedron Lett., 33:8087, 1992.
Brown and Yamaichi, Chem. Commun., 100, 1979.
Corey and Boaz, Tetrahedron Lett., 26:6019, 1985.
Corey and Ensley, J. Am. Chem. Soc., 97:6908, 1975.
Davis and Sheppard, Tetrahedron, 45:5703, 1989.
Deng et al., Org. Chem., 64:202, 1999.
Dess and Martin, J. Am. Chem. Soc., 113:7277, 1991.
Duhamel et al., Chem. Soc. Perkin Trans., 21:2509, 1993.
Evans, et al., In: Cancer Principles and Practice of Oncology, Devita et al., (eds.), Lippincot-Raven, N.Y., 1054-1087, 1997.
Flavell, Hematol. Oncol., 14(2):67-82, 1996.
Foon et al., Leukemia, 6(4):26-32, 1992.
Freedman et al., Hematol. Oncol. Clinics of N. Am., 4(2):405-456, 1990.
Giovanella et al., Cancer, 52(7):1146-1152, 1983.
Giuliani et al., Int. J. Cancer, January 15;27(1):5-13, 1981.
Gore and Vederas, J. Org. Chem., 51:3700, 1986.
Guo and Fuchs, Tetrahedron Lett., 39:1099, 1998.
Guo et al., Bioorg. Med. Chem. Lett., 9:419, 1999.
Inoue et al., Cancer Chemother. Pharmacol., 10(3):182-186, 1983.
Jacobson et al., Cell, 88:347-354, 1997.
Jiang et al., Liebigs Ann. Chem., 975, 1992.
Kessar and Rampal, Tetrahedron, 24:887, 1968.
Kessar et al., Tetrahedron, 24:893, 1968.
Khleif and Curt, In: Cancer Medicine, 4^{th} Ed., 855-868, 1997.
Kim et al., J. Am. Chem. Soc., 121:2056, 1999.
Korc, Surg. Oncol. Clin. N. Am., 7:25-41, 1998.
Kubo et al., Phytochemitstry, 31:3969, 1992.
Kuroda et al., J. Nat. Prod., 64(1):88-91, 2001.
LaCour et al., J. Am. Chem. Soc., 120:692, 1998.
Lipshutz et al., Tetrahedron Lett., 26:705, 1985.
Lipshutz et al., Tetrahedron, 40:5005, 1984.
Lipshutz, Synthesis, 87:325, 1987.
Ma et al., Carbohydr. Res., 329(3):495-505, 2000.
Ma et al., Bioorg. Med. Chem. Lett., 11(16):2153-2156, 2001a.
Ma et al., Carbohydr. Res., 334(2):159-164, 2001b.
Mimaki et al., Bioorganic Med. Chem. Lett., 7:633, 1997.
Morzycki et al., Tetrahedron Lett., 41:3751, 2000.
Moyano et al., J. Org. Chem., 52:2919, 1987.
Nicolaou et al., J. Am. Chem. Soc., 120:8674, 1998.
Nicolaou et al., Tetrahedron, 53:8751, 1997.
Pettit et al., J. Am. Chem. Soc., 110:2006, 1988.
Quesnel et al., Synlett, 413, 1998.
Ravindranath and Morton, Intern. Rev. Immunol., 7: 303-329, 1991.
Reich and Sikorski, J. Org. Chem., 64:14, 1999.
Remington's Pharmaceutical Sciences, 15^{th} Edition, 1035-1038; 1570-1580.
Rosenberg et al., N. Engl. J. Med., 319:1676, 1988.
Rosenberg et al., Ann. Surg. 210(4):474-548, 1989.
Rygaard and Povlsen, Acta Pathol. Microbiol. Scand., 77(4):758-760, 1969.
Schlosser and Strunk, Tetrahedron Lett., 25:741, 1984.
Schmuffand Trost, J. Org. Chem., 48:1404, 1983.
Schwesinger, Angew. Chem. Int. Ed. Engl., 26:1164, 1987.
Snider and Shi, Tetrahedron, 55:14823, 1999.
Stork and Hudrlik, J. Am. Chem. Soc., 90:4464, 1968.
Tashiro et al., Cancer Chemother., Pharmacol., 24(3):187-192, 1989.
Yu and Jin, J. Am. Chem. Soc., 122:9840, 2000.
Yu and Jin, J. Am. Chem. Soc., 123:3369, 2001.
Yu and Jin, Tetrahedron Lett., 42:369, 2001.
Yu et al., J. Am. Chem. Soc., 124(23):6576-6583, 2002.

1. A method of treating a subject with a pancreatic cancer, a leukemia, a colon cancer, a glioma or an ovarian cancer comprising administering a therapeutically effective amount of a pharmaceutical composition comprising orsaponin or a derivative thereof wherein said orsaponin has the molecular formula: 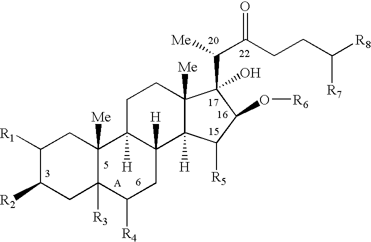
wherein,
R₁ is a H, an OH, or an MeO, with either an R or an S stereochemistry,
R₂ is a H, an OH, an ester or an amide,
R₃ is H, OH, or forms part of a double-bond A,
R₄ is H, OH, or forms part of a double-bond A,
R₅ is a H, a disaccharide, a monosaccharide or a trisaccharide,
R₆ is a disaccharide, a monosaccharide or a trisaccharide,
R₇ is a Me, a C₁₋₁₂ alkyl, or preferably a C₂₋₆ alkyl,
R₈ is a Me, a C₁₋₁₂ alkyl, or preferably a C₂₋₆ alkyl, and C₂₀ is an S or an R isomer,
or is a stereoisomer thereof.

2. The method of claim 1, wherein the orsaponin has the molecular formula:

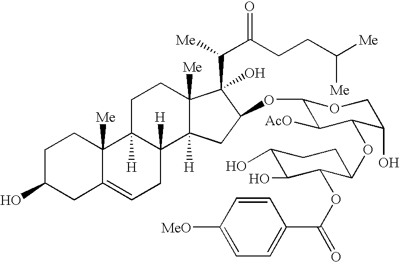

3. The method of claim 1, wherein the orsaponin derivative is 17-deoxyorsaponin.

4. The method of claim 1, wherein the leukemia is a chronic lymphocytic leukemia (CLL), or acute myeloid leukemia.

5. The method of claim 1, wherein the pancreatic cancer, leukemia, colon cancer, or ovarian cancer is a drug-resistant cancer.

6. The method of claim 1, wherein the pancreatic cancer, the leukemia, cancer, colon cancer, or the ovarian cancer is a metastatic cancer.

7. The method of claim 1, wherein the pancreatic cancer is a ductal adenocarcinoma, a mucinous cystadenocarcinoma, an acinar carcinoma, an unclassified large cell carcinoma, a small cell carcinoma, an intraductal papillary neoplasm, a mucinous cystadnoma, a papillary cystic neoplasm, or a pancreatoblastoma.

8. The method of claim 1, wherein the cancer exhibits constitutive NF-κε activity.

9. The method of claim 1, wherein the cancer has a p53 mutation or defect in p53 function.

10. The method of claim 1, wherein the ovarian cancer is a carcinoma, a serous cell cancer, a mucinous cell cancer, an endometrioid cell cancer, a clear cell cancer, a mesonephroid cell cancer, a Brenner cell cancer, or a mixed epithelial cell cancer.

11. The method of claim 1, wherein the therapeutically effective amount is 0.5-50 μg/kg/day.

12. The method of claim 11, wherein said therapeutically effective amount is 1-10 μg/kg/day.

13. The method of claim 1, wherein said orsaponin composition is administered systemically, regionally or locally.

14. The method of claim 13, wherein said orsaponin composition is administered by intravenous, intraartetial, intraperitoneal, intradermal, intratumoral, intramuscular, subcutaneous, oral, dermal, nasal, buccal, rectal, vaginal, inhalation, or topical administration.

15. The method of claim 1, further comprising treating the subject with a second anti-cancer agent.

16. The method of claim 15, wherein the second agent is a chemotherapeutic agent, a therapeutic antibody, a therapeutic polypeptide, a nucleic acid encoding a therapeutic polypeptide, a therapeutic nucleic acid encoding an antisense, a ribozyme or a RNA, a hormonal agent, an immunotherapeutic agent, or a radiotherapeutic agent.

17. The method of claim 15, wherein the second agent is administered simultaneously with the orsaponin composition.

18. The method of claim 15, wherein the second agent is administered prior to administration of the orsaponin composition.

19. The method of claim 15, wherein the second agent is administered after administration of the orsaponin composition.

20. The method of claim 1, wherein the subject is a mammal.

21. The method of claim 20, wherein the mammal is a human.

22. A method of inducing cytotoxicity in a pancreatic cancer cell, a leukemia cancer cell, a colon cancer cell, a glioma cancer cell, or an ovarian cancer cell, comprising contacting said cell with a pharmaceutical composition of orsaponin or a derivative thereof wherein said orsaponin has the molecular formula: 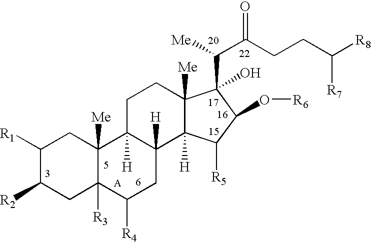
wherein,
R₁ is a H, an OH, or an MeO, with either an R or an S stereochemistry,
R₂ is a H, an OH, an ester or an amide,
R₃ is H, OH, or forms part of a double-bond A,
R₄ is H, OH, or forms part of a double-bond A,
R₅ is a H, a disaccharide, a monosaccharide or a trisaccharide,
R₆ is a disaccharide, a monosaccharide or a trisaccharide,
R₇ is a Me, a C₁₋₁₂ alkyl, or preferably a C₂₋₆ alkyl,
R₈ is a Me, a C₁₋₁₂ alkyl, or preferably a C₂₋₆ alkyl,
and C₂₀ is an S or an R isomer,
or is a stereoisomer thereof.

23. The method of claim 22, wherein the orsaponin derivative is 17-deoxyorsaponin.

24. The method of claim 22, wherein the leukemia is chronic lymphocytic leukemia (CLL), or acute myeloid leukemia.

25. The method of claim 22, wherein the pancreatic cancer cell, the leukemia cell, colon cancer cell, glioma cancer cell, or ovarian cancer cell is a metastatic cell.

26. The method of claim 22, wherein the pancreatic cancer cell, the leukemia cell, colon cancer cell, glioma cancer cell, or ovarian cancer cell is a drug resistant cell.

27. The method of claim 22, wherein the pancreatic cancer cell is a ductal adenocarcinoma cell, a mucinous cystadenocarcinoma cell, an acinar carcinoma cell, an unclassified large cell carcinoma cell, a small cell carcinoma cell, a pancreatoblastoma cell, an intraductal papillary neoplasm cell, a mucinous cystadnoma cell, or a papillary cystic neoplasm cell.

28. The method of claim 22, wherein the ovarian cancer cell is an carcinoma cell, a serous cell, a mucinous cell, an endometrioid cell, a clear cell mesonephroid cell, a Brenner cell, or a mixed epithelial cell.

29. The method of claim 22, wherein the orsaponin composition has an IC₅₀ of 0.1-10 nM.

30. The method of claim 29, wherein the orsaponin composition has an IC₅₀ of 0.1-5 nM.

31. The method of claim 30, wherein the orsaponin composition has an IC₅₀ of 0.1-1 nM.

32. The method of claim 31, wherein the orsaponin composition has an IC₅₀ of less than 1 nM.

33. The method of claim 22, wherein the cancer cell expresses NF-κβ.

34. The method of claim 22, wherein the cancer cell has a p53 mutation or defect in p53 function.

35. The method of claim 22, further comprising inducing apoptosis.

36. The method of claim 22, further comprising killing the pancreatic cell, the leukemia cell, a colon cancer cell, glioma cancer cell, or the ovarian cancer cell.

37. A method of inhibiting cell division of a pancreatic cancer cell, a leukemia cell, a colon cancer cell, glioma cancer cell, or an ovarian cancer cell, comprising contacting said cell with a pharmaceutical composition comprising orsaponin or a derivative thereof wherein said orsaponin has the molecular formula: 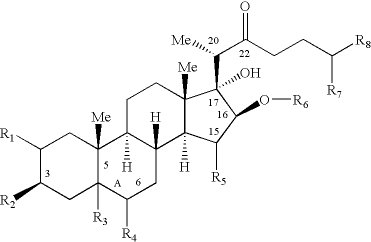
wherein,
R₁ is a H, an OH, or an MeO, with either an R or an S stereochemistry,
R₂ is a H, an OH, an ester or an amide,
R₃ is H, OH, or forms part of a double-bond A,
R₄ is H, OH, or forms part of a double-bond A,
R₅ is a H, a disaccharide, a monosaccharide or a trisaccharide,
R₆ is a disaccharide, a monosaccharide or a trisaccharide,
R₇ is a Me, a C₁₋₁₂ alkyl, or preferably a C₂₋₆ alkyl,
R₈ is a Me, a C₁₋₁₂ alkyl, or preferably a C₂₋₆ alkyl,
and C₂₀ is an S or an R isomer,
or is a stereoisomer thereof.

38. The method of claim 37, wherein the orsaponin derivative is 17-deoxyorsaponin.

39. The method of claim 37, wherein the leukemia is a chronic lymphocytic leukemia (CLL), or acute myeloid leukemia.

40. A method of inhibiting the growth of a pancreatic cancer cell, a leukemia cell, a colon cancer cell, glioma cancer cell, or an ovarian cancer cell, comprising contacting said cell with a pharmaceutical composition comprising orsaponin wherein said orsaponin has the molecular formula: 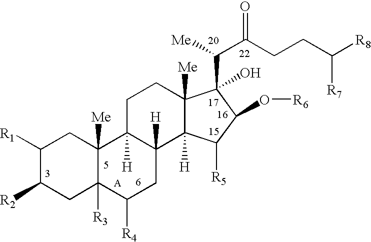
wherein,
R₁ is a H, an OH, or an MeO, with either an R or an S stereochemistry,
R₂ is a H, an OH, an ester or an amide,
R₃ is H, OH, or forms part of a double-bond A,
R₄ is H, OH, or forms part of a double-bond A,
R₅ is a H, a disaccharide, a monosaccharide or a trisaccharide,
R₆ is a disaccharide, a monosaccharide or a trisaccharide,
R₇ is a Me, a C₁₋₁₂ alkyl, or preferably a C₂₋₆ alkyl,
R₈ is a Me, a C₁₋₁₂ alkyl, or preferably a C₂₋₆ alkyl,
and C₂₀ is an S or an R isomer,
or is a stereoisomer thereof.

41. The method of claim 40, wherein the orsaponin derivative is 17-deoxyorsaponin.

42. The method of claim 40, wherein the leukemia is a chronic lymphocytic leukemia (CLL), or acute myeloid leukemia.

43. The method of claim 40, wherein the growth is metastatic growth.
